SEQUENCE LISTING (168 sequences)

SEQ ID NO: 1-5 - skipped (5 entries)

SEQ ID NO: 6 - protein, 4 aa, synthetic construct
RRRR

SEQ ID NO: 7 - protein, 5 aa, synthetic construct
RRRRR

SEQ ID NO: 8 - protein, 6 aa, synthetic construct
RRRRRR

SEQ ID NO: 9 - protein, 7 aa, synthetic construct
RRRRRRR

SEQ ID NO: 10 - protein, 8 aa, synthetic construct
RRRRRRRR

SEQ ID NO: 11 - protein, 9 aa, synthetic construct
RRRRRRRRR

SEQ ID NO: 12 - protein, 10 aa, synthetic construct
RRRRRRRRRR

SEQ ID NO: 13 - protein, 11 aa, synthetic construct
RRRRRRRRRRR

SEQ ID NO: 14 - protein, 12 aa, synthetic construct
RRRRRRRRRRRR

SEQ ID NO: 15 - protein, 13 aa, synthetic construct
RRRRRRRRRRRRR

SEQ ID NO: 16 - protein, 14 aa, synthetic construct
RRRRRRRRRRRRRR

SEQ ID NO: 17 - protein, 15 aa, synthetic construct
RRRRRRRRRRRRRRR

SEQ ID NO: 18 - protein, 16 aa, synthetic construct
RRRRRRRRRRRRRRRR

SEQ ID NO: 19-21 - skipped (3 entries)

SEQ ID NO: 22 - protein, 4 aa, synthetic construct
KKKK

SEQ ID NO: 23 - protein, 5 aa, synthetic construct
KKKKK

SEQ ID NO: 24 - protein, 6 aa, synthetic construct
KKKKKK

SEQ ID NO: 25 - protein, 7 aa, synthetic construct
KKKKKKK

SEQ ID NO: 26 - protein, 8 aa, synthetic construct
KKKKKKKK

SEQ ID NO: 27 - protein, 9 aa, synthetic construct
KKKKKKKKK

SEQ ID NO: 28 - protein, 10 aa, synthetic construct
KKKKKKKKKK

SEQ ID NO: 29 - protein, 11 aa, synthetic construct
KKKKKKKKKKK

SEQ ID NO: 30 - protein, 12 aa, synthetic construct
KKKKKKKKKKKK

SEQ ID NO: 31 - protein, 13 aa, synthetic construct
KKKKKKKKKKKKK

SEQ ID NO: 32 - protein, 14 aa, synthetic construct
KKKKKKKKKKKKKK

SEQ ID NO: 33 - protein, 15 aa, synthetic construct
KKKKKKKKKKKKKKK

SEQ ID NO: 34 - protein, 16 aa, synthetic construct
KKKKKKKKKKKKKKKK

SEQ ID NO: 35-37 - skipped (3 entries)

SEQ ID NO: 38 - protein, 4 aa, synthetic construct
HHHH

SEQ ID NO: 39 - protein, 5 aa, synthetic construct
HHHHH

SEQ ID NO: 40 - protein, 6 aa, synthetic construct
HHHHHH

SEQ ID NO: 41 - protein, 7 aa, synthetic construct
HHHHHHH

SEQ ID NO: 42 - protein, 8 aa, synthetic construct
HHHHHHHH

SEQ ID NO: 43 - protein, 9 aa, synthetic construct
HHHHHHHHH

SEQ ID NO: 44 - protein, 10 aa, synthetic construct
HHHHHHHHHH

SEQ ID NO: 45 - protein, 11 aa, synthetic construct
HHHHHHHHHHH

SEQ ID NO: 46 - protein, 12 aa, synthetic construct
HHHHHHHHHHHH

SEQ ID NO: 47 - protein, 13 aa, synthetic construct
HHHHHHHHHHHHH

SEQ ID NO: 48 - protein, 14 aa, synthetic construct
HHHHHHHHHHHHHH

SEQ ID NO: 49 - protein, 15 aa, synthetic construct
HHHHHHHHHHHHHHH

SEQ ID NO: 50 - protein, 16 aa, synthetic construct
HHHHHHHHHHHHHHHH

SEQ ID NO: 51-53 - skipped (3 entries)

SEQ ID NO: 54 - protein, 4 aa, synthetic construct
  SITE 1..4: note = D-Arginine
RRRR

SEQ ID NO: 55 - protein, 5 aa, synthetic construct
  SITE 1..5: note = D-Arginine
RRRRR

SEQ ID NO: 56 - protein, 6 aa, synthetic construct
  SITE 1..6: note = D-Arginine
RRRRRR

SEQ ID NO: 57 - protein, 7 aa, synthetic construct
  SITE 1..7: note = D-Arginine
RRRRRRR

SEQ ID NO: 58 - protein, 8 aa, synthetic construct
  SITE 1..8: note = D-Arginine
RRRRRRRR

SEQ ID NO: 59 - protein, 9 aa, synthetic construct
  SITE 1..9: note = D-Arginine
RRRRRRRRR

SEQ ID NO: 60 - protein, 10 aa, synthetic construct
  SITE 1..10: note = D-Arginine
RRRRRRRRRR

SEQ ID NO: 61 - protein, 11 aa, synthetic construct
  SITE 1..11: note = D-Arginine
RRRRRRRRRRR

SEQ ID NO: 62 - protein, 12 aa, synthetic construct
  SITE 1..12: note = D-Arginine
RRRRRRRRRRRR

SEQ ID NO: 63 - protein, 13 aa, synthetic construct
  SITE 1..13: note = D-Arginine
RRRRRRRRRRRRR

SEQ ID NO: 64 - protein, 14 aa, synthetic construct
  SITE 1..14: note = D-Arginine
RRRRRRRRRRRRRR

SEQ ID NO: 65 - protein, 15 aa, synthetic construct
  VARIANT 1
RRRRRRRRRRRRRRR

SEQ ID NO: 66 - protein, 16 aa, synthetic construct
  SITE 1..16: note = D-Arginine
RRRRRRRRRRRRRRRR

SEQ ID NO: 67-69 - skipped (3 entries)

SEQ ID NO: 70 - protein, 4 aa, synthetic construct
  SITE 1..4: note = D-Lysine
KKKK

SEQ ID NO: 71 - protein, 5 aa, synthetic construct
  SITE 1..5: note = D-Lysine
KKKKK

SEQ ID NO: 72 - protein, 6 aa, synthetic construct
  SITE 1..6: note = D-Lysine
KKKKKK

SEQ ID NO: 73 - protein, 7 aa, synthetic construct
  SITE 1..7: note = D-Lysine
KKKKKKK

SEQ ID NO: 74 - protein, 8 aa, synthetic construct
  SITE 1..8: note = D-Lysine
KKKKKKKK

SEQ ID NO: 75 - protein, 9 aa, synthetic construct
  SITE 1..9: note = D-Lysine
KKKKKKKKK

SEQ ID NO: 76 - protein, 10 aa, synthetic construct
  SITE 1..10: note = D-Lysine
KKKKKKKKKK

SEQ ID NO: 77 - protein, 11 aa, synthetic construct
  SITE 1..11: note = D-Lysine
KKKKKKKKKKK

SEQ ID NO: 78 - protein, 12 aa, synthetic construct
  SITE 1..12: note = D-Lysine
KKKKKKKKKKKK

SEQ ID NO: 79 - protein, 13 aa, synthetic construct
  SITE 1..13: note = D-Lysine
KKKKKKKKKKKKK

SEQ ID NO: 80 - protein, 14 aa, synthetic construct
  SITE 1..14: note = D-Lysine
KKKKKKKKKKKKKK

SEQ ID NO: 81 - protein, 15 aa, synthetic construct
  SITE 1..15: note = D-Lysine
KKKKKKKKKKKKKKK

SEQ ID NO: 82 - protein, 16 aa, synthetic construct
  SITE 1..16: note = D-Lysine
KKKKKKKKKKKKKKKK

SEQ ID NO: 83-85 - skipped (3 entries)

SEQ ID NO: 86 - protein, 4 aa, synthetic construct
  SITE 1..4: note = D-Histidine
HHHH

SEQ ID NO: 87 - protein, 5 aa, synthetic construct
  SITE 1..5: note = D-Histidine
HHHHH

SEQ ID NO: 88 - protein, 6 aa, synthetic construct
  SITE 1..6: note = D-Histidine
HHHHHH

SEQ ID NO: 89 - protein, 7 aa, synthetic construct
  SITE 1..7: note = D-Histidine
HHHHHHH

SEQ ID NO: 90 - protein, 8 aa, synthetic construct
  SITE 1..8: note = D-Histidine
HHHHHHHH

SEQ ID NO: 91 - protein, 9 aa, synthetic construct
  SITE 1..9: note = D-Histidine
HHHHHHHHH

SEQ ID NO: 92 - protein, 10 aa, synthetic construct
  SITE 1..10: note = D-Histidine
HHHHHHHHHH

SEQ ID NO: 93 - protein, 11 aa, synthetic construct
  SITE 1..11: note = D-Histidine
HHHHHHHHHHH

SEQ ID NO: 94 - protein, 12 aa, synthetic construct
  SITE 1..12: note = D-Histidine
HHHHHHHHHHHH

SEQ ID NO: 95 - protein, 13 aa, synthetic construct
  SITE 1..13: note = D-Histidine
HHHHHHHHHHHHH

SEQ ID NO: 96 - protein, 14 aa, synthetic construct
  SITE 1..14: note = D-Histidine
HHHHHHHHHHHHHH

SEQ ID NO: 97 - protein, 15 aa, synthetic construct
  SITE 1..15: note = D-Histidine
HHHHHHHHHHHHHHH

SEQ ID NO: 98 - protein, 16 aa, synthetic construct
  SITE 1..16: note = D-Histidine
HHHHHHHHHHHHHHHH

SEQ ID NO: 99-101 - skipped (3 entries)

SEQ ID NO: 102 - protein, 4 aa, synthetic construct
EEEE

SEQ ID NO: 103 - protein, 5 aa, synthetic construct
EEEEE

SEQ ID NO: 104 - protein, 6 aa, synthetic construct
EEEEEE

SEQ ID NO: 105 - protein, 7 aa, synthetic construct
EEEEEEE

SEQ ID NO: 106 - protein, 8 aa, synthetic construct
EEEEEEEE

SEQ ID NO: 107 - protein, 9 aa, synthetic construct
EEEEEEEEE

SEQ ID NO: 108 - protein, 10 aa, synthetic construct
EEEEEEEEEE

SEQ ID NO: 109 - protein, 11 aa, synthetic construct
EEEEEEEEEEE

SEQ ID NO: 110 - protein, 12 aa, synthetic construct
EEEEEEEEEEEE

SEQ ID NO: 111 - protein, 13 aa, synthetic construct
EEEEEEEEEEEEE

SEQ ID NO: 112 - protein, 14 aa, synthetic construct
EEEEEEEEEEEEEE

SEQ ID NO: 113 - protein, 15 aa, synthetic construct
EEEEEEEEEEEEEEE

SEQ ID NO: 114 - protein, 16 aa, synthetic construct
EEEEEEEEEEEEEEEE

SEQ ID NO: 115-117 - skipped (3 entries)

SEQ ID NO: 118 - protein, 4 aa, synthetic construct
DDDD

SEQ ID NO: 119 - protein, 5 aa, synthetic construct
DDDDD

SEQ ID NO: 120 - protein, 6 aa, synthetic construct
DDDDDD

SEQ ID NO: 121 - protein, 7 aa, synthetic construct
DDDDDDD

SEQ ID NO: 122 - protein, 8 aa, synthetic construct
DDDDDDDD

SEQ ID NO: 123 - protein, 9 aa, synthetic construct
DDDDDDDDD

SEQ ID NO: 124 - protein, 10 aa, synthetic construct
DDDDDDDDDD

SEQ ID NO: 125 - protein, 11 aa, synthetic construct
DDDDDDDDDDD

SEQ ID NO: 126 - protein, 12 aa, synthetic construct
DDDDDDDDDDDD

SEQ ID NO: 127 - protein, 13 aa, synthetic construct
DDDDDDDDDDDDD

SEQ ID NO: 128 - protein, 14 aa, synthetic construct
DDDDDDDDDDDDDD

SEQ ID NO: 129 - protein, 15 aa, synthetic construct
DDDDDDDDDDDDDDD

SEQ ID NO: 130 - protein, 16 aa, synthetic construct
DDDDDDDDDDDDDDDD

SEQ ID NO: 131-133 - skipped (3 entries)

SEQ ID NO: 134 - protein, 4 aa, synthetic construct
  SITE 1..4: note = D-glutamic acid
EEEE

SEQ ID NO: 135 - protein, 5 aa, synthetic construct
  SITE 1..5: note = D-glutamic acid
EEEEE

SEQ ID NO: 136 - protein, 6 aa, synthetic construct
  SITE 1..6: note = D-Glutamic acid
EEEEEE

SEQ ID NO: 137 - protein, 7 aa, synthetic construct
  SITE 1..7: note = D-Glutamic acid
EEEEEEE

SEQ ID NO: 138 - protein, 8 aa, synthetic construct
  SITE 1..8: note = D-Glutamic acid
EEEEEEEE

SEQ ID NO: 139 - protein, 9 aa, synthetic construct
  SITE 1..9: note = D-Glutamic acid
EEEEEEEEE

SEQ ID NO: 140 - protein, 10 aa, synthetic construct
  SITE 1..10: note = D-Glutamic acid
EEEEEEEEEE

SEQ ID NO: 141 - protein, 11 aa, synthetic construct
  SITE 1..11: note = D-Glutamic acid
EEEEEEEEEEE

SEQ ID NO: 142 - protein, 12 aa, synthetic construct
  SITE 1..12: note = D-Glutamic acid
EEEEEEEEEEEE

SEQ ID NO: 143 - protein, 13 aa, synthetic construct
  SITE 1..13: note = D-Glutamic acid
EEEEEEEEEEEEE

SEQ ID NO: 144 - protein, 14 aa, synthetic construct
  SITE 1..14: note = D-Glutamic acid
EEEEEEEEEEEEEE

SEQ ID NO: 145 - protein, 15 aa, synthetic construct
  SITE 1..15: note = D-Glutamic acid
EEEEEEEEEEEEEEE

SEQ ID NO: 146 - protein, 16 aa, synthetic construct
  SITE 1..16: note = D-Glutamic acid
EEEEEEEEEEEEEEEE

SEQ ID NO: 147-149 - skipped (3 entries)

SEQ ID NO: 150 - protein, 4 aa, synthetic construct
  SITE 1..4: note = D-Aspartic acid
DDDD

SEQ ID NO: 151 - protein, 5 aa, synthetic construct
  SITE 1..5: note = D-Aspartic acid
DDDDD

SEQ ID NO: 152 - protein, 6 aa, synthetic construct
  SITE 1..6: note = D-Aspartic acid
DDDDDD

SEQ ID NO: 153 - protein, 7 aa, synthetic construct
  SITE 1..7: note = D-Aspartic acid
DDDDDDD

SEQ ID NO: 154 - protein, 8 aa, synthetic construct
  SITE 1..8: note = D-Aspartic acid
DDDDDDDD

SEQ ID NO: 155 - protein, 9 aa, synthetic construct
  SITE 1..9: note = D-Aspartic acid
DDDDDDDDD

SEQ ID NO: 156 - protein, 10 aa, synthetic construct
  SITE 1..10: note = D-Aspartic acid
DDDDDDDDDD

SEQ ID NO: 157 - protein, 11 aa, synthetic construct
  SITE 1..11: note = D-Aspartic acid
DDDDDDDDDDD

SEQ ID NO: 158 - protein, 12 aa, synthetic construct
  SITE 1..12: note = D-Aspartic acid
DDDDDDDDDDDD

SEQ ID NO: 159 - protein, 13 aa, synthetic construct
  SITE 1..13: note = D-Aspartic acid
DDDDDDDDDDDDD

SEQ ID NO: 160 - protein, 14 aa, synthetic construct
  SITE 1..14: note = D-Aspartic acid
DDDDDDDDDDDDDD

SEQ ID NO: 161 - protein, 15 aa, synthetic construct
  SITE 1..15: note = D-Aspartic acid
DDDDDDDDDDDDDDD

SEQ ID NO: 162 - protein, 16 aa, synthetic construct
  SITE 1..16: note = D-Aspartic acid
DDDDDDDDDDDDDDDD

SEQ ID NO: 163 - protein, 28 aa, synthetic construct
ERERERERERERERERERERERERERER

SEQ ID NO: 164 - protein, 24 aa, synthetic construct
EEEEEEEEEEEERRRRRRRRRRRR

SEQ ID NO: 165 - protein, 12 aa, synthetic construct
RRRRRRRRRRRR

SEQ ID NO: 166 - DNA, 200 bp, unidentified
  source 1..200: mol_type = other DNA
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 167 - RNA, 720 nt, unidentified
  source 1..720: mol_type = mRNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 168 - DNA, 4848 bp, unidentified
  source 1..4848: mol_type = genomic DNA
gccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctggcagcggcagcggcagcggcagcaccgataccctgcaggcggaaaccgatcagctggaagatgaaaaatatgcgctgcagaccgaaattgcgaacctgctgaaagaaaaagaaaaactggaatttattatcgatggctaaggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctg

(19) United States
(12) Patent Application Publication
(10) Pub. No.: US 2025/0064951 A1
(43) Pub. Date: Feb. 27, 2025

(54) DENDRITIC POLYELECTROLYTE ZWITTERIONS, JANUS-TYPE CONJUGATE COMPRISING SAME AND USES THEREOF

(71) Applicant: ArtProBio Co., Ltd., Seoul (KR)

(72) Inventors: Yong-beom LIM, Seoul (KR); Youngjoo LEE, Seoul (KR); Youngjoo CHOI, Seoul (KR)

(73) Assignee: ArtProBio Co., Ltd., Seoul (KR)

(21) Appl. No.: 18/663,676

(22) Filed: May 14, 2024

(30) Foreign Application Priority Data
May 16, 2023 (KR) 10-2023-0062794
May 13, 2024 (KR) 10-2024-0062272

(51) Int. Cl. A61K 47/64; A61K 48/00

(52) U.S. Cl. CPC A61K 47/645; A61K 47/641; A61K 48/0041

(57) ABSTRACT

The present disclosure relates to a Janus-type dendritic polyelectrolyte zwitterion as a non-fouling, non-toxic carrier with high intracellular delivery efficiency, and a use thereof. Since the Janus-type dendritic polyelectrolyte zwitterion according to the present disclosure is completely protected from the external environment, it self-assembles into a supramolecular structure in solution without an additional process. And, since it has no cytotoxicity and can efficiently carry a loaded drug or target gene into cells, it can be usefully used in various therapeutic drug or nucleic acid delivery systems, drug therapy technologies, cell marker systems, gene therapies, etc.

### TECHNICAL FIELD

The present disclosure relates to a dendritic polyelectrolyte zwitterion (DPZ), a Janus-type dendritic polyelectrolyte zwitterion (JDPZ), and uses thereof as a non-fouling, non-toxic carrier with high intracellular delivery efficiency.

### BACKGROUND ART

Ionic macromolecules include polyelectrolytes and zwitterions. They have opposite solution behaviors. Polyelectrolytes are polymers composed of repeating units with positive or negative charges. They exist as an expanded structure in deionized water (DW) due to the repulsion effect between the same charges, but as the salt increases, they collapse and become less soluble in water. This is called the polyelectrolyte effect.

Zwitterions have the same number of positive and negative charges in one molecule and, thus, the net charge is 0. Due to the attraction between positive and negative charges within the molecule, they exist in a collapsed form in deionized water (DW), but as the salt increases, the molecules spread apart from each other, and as a result, they become highly soluble in water. This is called the anti-polyelectrolyte effect.

Because cell membranes have a negative charge, most drug carriers are being developed to have positive charges. These positively charged polymers or nanoparticles have the advantage of forming a strong bond with the negatively charged cell membrane and being effectively delivered into the cell, but they have the problems that they are highly toxic, can trigger an immune response, and agglomerate with each other under ionic strength conditions similar to those in the body due to the polyelectrolyte effect. Furthermore, the adsorption of serum/cellular proteins on the surface of nanoparticles (fouling) can lead to the aggregation of nanoparticles and alter their biological functions.

Therefore, there is an urgent need for a new structure as a drug carrier that has high non-fouling property, high intracellular delivery efficiency, low toxicity, and high structural stability at physiological salt concentrations.

### REFERENCES OF RELATED ART

### Patent Documents

(Patent document 1) Patent document 1. Korean Patent Registration No. 10-2610361.

### DISCLOSURE

### Technical Problem

The present disclosure was developed in consideration of the above problems and is directed to providing a dendritic polyelectrolyte zwitterion for preparing dendritic compounds and nanostructures with high non-fouling property, high intracellular delivery efficiency, low toxicity, and high structural stability at physiological salt concentrations.

The present disclosure is also directed to providing a Janus-type dendritic polyelectrolyte zwitterion (JDPZ) containing the hydrophilic dendritic polyelectrolyte zwitterion (DPZ) and a hydrophobic compound.

The present disclosure is also directed to providing a composition that can safely store hydrophobic drugs and genetic materials and can deliver them into cells.

### Technical Solution

Provided is a polyelectrolyte-zwitterion-based dendritic compound represented by the following General Formula 1 or 2.

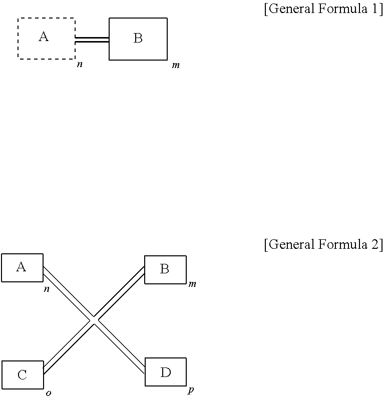

In the above formulas,
each of A and C is independently at least one selected from a group consisting of arginine (Arg, R), histidine (His, H), lysine (Lys, K), ornithine (Orn) and guanidine,
each of B and D is independently at least one selected from a group consisting of glutamic acid (Glu, E) and aspartic acid (Asp, D),
each of n, m, o and p is independently an integer selected from 3 to 16, and
each of the arginine (Arg, R), histidine (His, H), lysine (Lys, K), ornithine (Orn), guanidine, glutamic acid (Glu, E) and aspartic acid (Asp, D) is independently an L- or D-amino acid.

The dendritic compound is characterized in that n, m, o and p are all the same under some conditions between pH 2 and pH 13.

The present disclosure also provides a dendritic polyelectrolyte zwitterion, which contains a 1st to 5th generation dendrimer-based core; and a positively charged peptide represented by SEQ ID NO: 1 and/or a negatively charged peptide represented by SEQ ID NO: 2 bound to the terminal functional group of the dendrimer core.

[SEQ ID NO: 1]
[X₁]ₙ
[SEQ ID NO: 2]
[X₂]ₙ

In the above sequences, X₁ is any one selected from arginine (Arg, R), histidine (His, H), lysine (Lys, K), ornithine (Orn) and guanidine, and X₂ is glutamic acid (Glu, E) or aspartic acid (Asp, D),
n is an integer from 3 to 16, and
each of the arginine (Arg, R), histidine (His, H), lysine (Lys, K), ornithine (Orn), guanidine, glutamic acid (Glu, E) and aspartic acid (Asp, D) is independently an L- or D-amino acid.

The dendrimer-based core may be, for example, any one selected from a group consisting of a polyamidoamine (PAMAM) dendrimer, a polylysine dendrimer, a polyimine (PI) dendrimer, a polypropyleneimine (PPI) dendrimer, a polyester dendrimer, a polyamide dendrimer, a polyurethane dendrimer, a polyornithine dendrimer, a carbosilane dendrimer, a polyether dendrimer, a polyglutamic acid dendrimer, a polyaspartic acid dendrimer, a polyglycerol dendrimer and a polymelamine dendrimer, although it is not particularly limited as long as it contains a terminal functional group capable of binding to a positively charged peptide and/or a negatively charged peptide.

The dendrimer-based core may be of the 1st or 2nd generation.

The same number of the positively charged peptide represented by SEQ ID NO: 1 and the negatively charged peptide represented by SEQ ID NO: 2 may be bound to the terminal of the dendrimer-based core.

If the dendrimer-based core is of the 1st generation, one strand of the positively charged peptide chain and one strand of the negatively charged peptide chain may be bound, and if the dendrimer-based core is of the 2nd generation, two strands of the positively charged peptide chain and two strands of the negatively charged peptide chain may be bound.

The positively charged peptide may be represented by any one selected from a group consisting of SEQ ID NOS: 3 to 86.

The negatively charged peptide may be represented by any one selected from a group consisting of SEQ ID NOS: 87 to 142.

The present disclosure also provides a Janus-type dendritic polyelectrolyte zwitterion, which contains a 1st to 5th generation dendrimer-based core; a plurality of positively charged peptides represented by SEQ ID NO: 1 and a plurality of negatively charged peptides represented by SEQ ID NO: 2 alternately bonded to the functional group of one terminal, and a hydrophobic compound or an amino acid derivative bound to the functional group of the other terminal based on the dendrimer core.

[SEQ ID NO: 1]
[X₁]ₙ
[SEQ ID NO: 2]
[X₂]ₙ

In the above sequences, X₁ is any one selected from arginine (Arg, R), histidine (His, H), lysine (Lys, K), ornithine (Orn) and guanidine, and X₂ is glutamic acid (Glu, E) or aspartic acid (Asp, D), and n is an integer from 3 to 16.

The dendrimer-based core may be of the 2nd or 3rd generation.

The same number of the positively charged peptide represented by SEQ ID NO: 1 and the negatively charged peptide represented by SEQ ID NO: 2 may be bound to the functional group at one terminal of the dendrimer-based core.

The dendrimer-based core has 2^{x} (x is the generation number) terminal functional groups on the surface. Among the total terminal functional groups present in the dendrimer-based core, half may be bound to the peptide represented by SEQ ID NO: 1 or 2 and the other half may be bound independently to the hydrophobic compound or the amino acid derivative.

The positively charged peptide may be represented by any one selected from a group consisting of SEQ ID NOS: 3 to 86.

The negatively charged peptide may be represented by any one selected from a group consisting of SEQ ID NOS: 87 to 142.

The dendrimer is characterized in that the number of positively charged peptides and the number of negatively charged peptides are equal under some conditions between pH 2 and pH 13.

The hydrophobic compound may be any one selected from a group consisting of a fatty acid having 3 to 30 carbon atoms, a hydrophobic drug, and a fluorescent material.

The present disclosure also provides a nanostructure containing the Janus-type dendritic polyelectrolyte zwitterion.

The nanostructure may be an elongated fiber structure in the form of a nanofiber.

The average diameter of the nanostructure may be 8 to 30 nm.

The present disclosure also provides a gene or drug carrier containing: the Janus-type dendritic polyelectrolyte zwitterion described above; and a target gene or a drug loaded in the dendritic polyelectrolyte zwitterion.

The gene may be a plasmid, an mRNA, an RNA, a DNA, or a combination thereof.

The drug may be a low-molecular-weight drug, a gene drug, a protein drug, an antibody drug, a synthetic compound drug, or a combination thereof.

### Advantageous Effects

The present disclosure provides a novel molecule in the form of a dendrimer composed of a positively charged peptide and a negatively charged peptide, which has the properties of a polyelectrolyte and a zwitterion at the same time. Its structure can be maintained stably in vivo since interaction with nucleic acids, proteins, etc. can be minimized due to its non-cytotoxicity and non-fouling property.

In addition, since the Janus-type dendritic polyelectrolyte zwitterion according to the present disclosure can form a complex with a target gene or a drug, a carrier with stable structure and excellent intracellular delivery efficiency can be provided.

Since the Janus-type dendritic polyelectrolyte zwitterion according to the present disclosure is completely protected from the external environment, it self-assembles into a supramolecular structure in solution without an additional process. And, since it has no cytotoxicity and can efficiently carry a loaded drug or target gene into cells, it can be usefully used in various therapeutic drug or nucleic acid delivery systems, drug therapy technologies, cell marker systems, gene therapies, etc.

### BRIEF DESCRIPTION OF DRAWINGS

FIG. 1 is a conceptual diagram showing the basic structure of dendritic polyelectrolyte zwitterions.

FIG. 2a is the MALDI-TOF MS spectrum of (+1)₂(−1)₂ prepared in Example 1-1, FIG. 2b is the MALDI-TOF MS spectrum of (+2)₂(−2)₂ prepared in Example 1-2, FIG. 2c is the MALDI-TOF MS spectrum of (+3)₂(−3)₂ prepared in Example 1-3, FIG. 2d is the MALDI-TOF MS spectrum of (+4)₂(−4)₂ prepared in Example 1-4, FIG. 2e is the MALDI-TOF MS spectrum of (+5)₂(−5)₂ prepared in Example 1-5, and FIG. 2f is the MALDI-TOF MS spectrum of (+6)₂(−6)₂ prepared in Example 1-6.

FIG. 3 shows a result of analyzing the cytotoxicity of dendritic polyelectrolyte zwitterions peptides prepared in Examples 1-1 to 1-6 (DPZ(+M)ₙ(−M)ₙ) and peptides prepared in Comparative Examples 3-1 and 3-2 depending on concentration (0, 2, 4, 8, 16, 32, 64 and 128 μM).

FIG. 4a shows an EMSA result of analyzing the non-fouling property of dendritic polyelectrolyte zwitterions of Examples 1-1 and 1-2 ((+1)₂(−1)₂ and (+2)₂(−2)₂) for proteins.

FIG. 4b shows an EMSA result of analyzing the non-fouling property of dendritic polyelectrolyte zwitterions of Examples 1-3 and 1-4 ((+3)₂(−3)₂ and (+4)₂(−4)₂) for proteins.

FIG. 4c shows an EMSA result of analyzing the non-fouling property of dendritic polyelectrolyte zwitterions of Examples 1-5 and 1-6 ((+5)₂(−5)₂ and (+6)₂(−6)₂) for proteins.

FIG. 5 shows an EMSA result of analyzing the non-fouling property of a linear peptide R12 (SEQ ID NO: 165) for proteins.

FIG. 6 shows an EMSA result of analyzing the interaction pattern between a dendritic polyelectrolyte zwitterion of Examples 1-6 ((+6)₂(−6)₂) and various genetic materials (single-stranded DNA, mRNA, and plasmid DNA).

FIG. 7 shows an EMSA result of analyzing complex formation between dendritic polyelectrolyte zwitterions of Examples 1-3 to 1-6 ((+3)₂(−3)₂, (+4)₂(−4)₂, (+5)₂(−5)₂ and (+6)₂(−6)₂) and mRNA depending on charge ratio.

FIGS. 8a to 8f show the MALDI-TOF spectra of DPZs prepared in Examples 2-1 to 2-6 into which the fluorescent material FAM is introduced. FIG. 8a: (+1)₂(−1)₂_FAM of Example 2-1, FIG. 8b: (+2)₂(−2)₂_FAM of Example 2-2, FIG. 8c: (+3)₂(−3)₂_FAM of Example 2-3, FIG. 8d: (+4)₂(−4)₂_FAM of Example 2-4, FIG. 8e: (+5)₂(−5)₂_FAM of Example 2-5, FIG. 8f: (+6)₂(−6)₂_FAM of Example 2-6.

FIG. 9 shows a result of analyzing the intracellular delivery efficiency of (+M)ₙ(−M)ₙ_FAM of Examples 2-1 to 2-6 in HeLa cells by flow cytometry depending on concentration (16 M (top), 32 M (bottom)).

FIGS. 10a to 10f show the MALDI-TOF spectra of G1 Janus-type dendritic polyelectrolyte zwitterions (JDPZ, (+M)₁(−M)₁ conjugate_1) prepared in Examples 3-1 to 3-6. FIG. 10a: (+1)₁(−1)₁ conjugate_1 of Example 3-1, FIG. 10b: (+2)₁(−2)₁ conjugate_1 of Example 3-2, FIG. 10c: (+3)₁(−3)₁ conjugate_1 of Example 3-3, FIG. 10d: (+4)₁(−4)₁ conjugate_1 of Example 3-4, FIG. 10e: (+5)₁(−5)₁ conjugate_1 of Example 3-5, FIG. 10f: (+6)₁(−6)₁ conjugate_1 of Example 3-6.

FIG. 11 shows the AFM images of Janus-type dendritic polyelectrolyte zwitterions of Examples 3-1 to 3-6 ((+1)₁(−1)₁-conjugate_1, (+2)₁(−2)₁-conjugate_1, (+3)₁(−3)₁-conjugate_1, (+4)₁(−4)₁-conjugate_1, (+5)₁(−5)₁-conjugate_1, and (+6)₁(−6)₁-conjugate_1) in DW solution.

FIG. 12 shows a result of analyzing the cytotoxicity of (+M)₁(−M)₁ conjugate_1 of Examples 3-1 to 3-6 depending on concentration (0, 4, 8, 16, 32, 64 μM).

FIGS. 13a to 131 show the MALDI-TOF spectra of G1 Janus-type dendritic polyelectrolyte zwitterions (JDPZs, (+M)₁(−M)₁ conjugate_FAM) prepared in Examples 4-1 to 4-12 into which a fluorescent material is introduced. FIG. 13a: (+1)₁(−1)₁ conjugate_FAM of Example 4-1, FIG. 13b: (+2)₁(−2)₁ conjugate_FAM of Example 4-2, FIG. 13c: (+3)₁(−3)₁ conjugate_FAM of Example 4-3, FIG. 13d: (+4)₁(−4)₁ conjugate_FAM of Example 4-4, FIG. 13e: (+5)₁(−5)₁ conjugate_FAM of Example 4-5, FIG. 13f: (+6)₁(−6)₁ conjugate_FAM of Example 4-6, FIG. 13g: (+7)₁(−7)₁ conjugate_FAM of Example 4-7, FIG. 13h: (+8)₁(−8)₁ conjugate_FAM of Example 4-8, FIG. 13i: (+5)₁(−5)₁ conjugate (RD)_FAM of Example 4-9, FIG. 13j: (+6)₁(−6)₁ conjugate (RD) FAM of Example 4-10, FIG. 13k: (+5)₁(−5)₁ conjugate (KE)_FAM of Example 4-11, FIG. 13l: (+6)₁(−6)₁ conjugate (KE)_FAM of Example 4-12.

FIGS. 14a to 14c show a result of analyzing the intracellular delivery efficiency (degree of internalization) of JDPZs ((+M)₁(−M)₁ conjugate_FAM) prepared in Examples 4-1 to 4-6 by flow cytometry.

FIG. 15 shows a result of analyzing the intracellular delivery efficiency of JDPZs prepared in Examples 4-6 to 4-12 ((+M)₁(−M)₁ conjugate_FAM) by flow cytometry.

FIGS. 16a to 16d show the MALDI-TOF spectra of G2 Janus-type dendritic polyelectrolyte zwitterions prepared in Examples 5-1 to 5-4 (JDPZ ((+M)₂(−M)₂ conjugate_1). FIG. 16a: (+3)₂(−3)₂ conjugate_1 prepared in Example 5-1, FIG. 16b: (+4)₂(−4)₂ conjugate_1 prepared in Example 5-2, FIG. 16c: (+5)₂(−5)₂ conjugate_1 prepared in Example 5-3, FIG. 16d: (+6)₂(−6)₂ conjugate_1 prepared in Example 5-4.

FIG. 17a shows the MALDI-TOF spectrum of (+3)₄ conjugate prepared in Comparative Example 1-1, FIG. 17b shows the MALDI-TOF spectrum of (+3)₂(Ø)₂ conjugate prepared in Comparative Example 1-2, and FIG. 17c shows the MALDI-TOF spectrum of (Ø)₂(−3)₂ conjugate prepared in Comparative Example 1-3.

FIGS. 18a to 18d show the AFM images of G2 Janus-type dendritic polyelectrolyte zwitterions of Examples 5-1 to 5-4 (JDPZs, (+M)₂(−M)₂ conjugate_1). FIG. 18a: (+3)₂(−3)₂ conjugate_1 prepared in Example 5-1, FIG. 18b: (+4)₂(−4)₂ conjugate_1 prepared in Example 5-2. FIG. 18c: (+5)₂(−5)₂ conjugate_1 prepared in Example 5-3, FIG. 18d: (+6)₂(−6)₂ conjugate_1 prepared in Example 5-4.

FIG. 19 shows a result of analyzing the cytotoxicity of a G2 Janus-type dendritic polyelectrolyte zwitterion prepared in Example 5-1 (JDPZ, (+M)₂(−M)₂ conjugate_1) and G2 Janus-type dendrimers prepared in Comparative Examples 1-1 to 1-3.

FIGS. 20a and 20b show the DLS spectra of a Janus-type dendritic polyelectrolyte zwitterion ((+6)₂(−6)₂ conjugate_1) of Example 5-4 (a) and a Janus-type dendrimer ((+3)₂(Ø3)₂ conjugate) of Comparative Example 1-2 (b) under different solution conditions (DW or PBS).

FIG. 21a shows an EMSA result of analyzing the non-fouling property of a Janus-type dendritic polyelectrolyte zwitterion (JDPZ ((+6)₂(−6)₂ conjugate_1)) of Example 5-4 and a Janus-type dendrimer ((+3)₂(Ø3)₂ conjugate) of Comparative Example 1-2 for proteins.

FIG. 21b shows an EMSA result of analyzing the non-fouling property of a Janus-type dendritic polyelectrolyte zwitterion (JDPZ ((+6)₂(−6)₂ conjugate_1)) of Example 5-4 and a Janus-type dendrimer ((+3)₂(Ø3)₂ conjugate) of Comparative Example 1-2 for genetic materials. ‘L’ is an RNA marker.

FIGS. 22a to 22d show the MALDI-TOF spectra of G2 Janus-type dendritic polyelectrolyte zwitterions (JDPZ ((+M)₂(−M)₂ conjugate_FAM) prepared in Examples 6-1 and 6-2 and Comparative Examples 2-1 and 2-2, into which a fluorescence material is introduced. FIG. 22a: (+3)₂(−3)₂ conjugate_FAM prepared in Example 6-1, FIG. 22b: (+6)₂(−6)₂ conjugate_FAM prepared in Example 6-2, FIG. 22c: (+3)₂(Ø3)₂ conjugate_FAM of Comparative Example 2-1, FIG. 22d: (Ø3)₂(−3)₂ conjugate_FAM of Comparative Example 2-2.

FIG. 23 shows a result of analyzing the intracellular delivery efficiency of JDPZ ((+6)₂(−6)₂ conjugate_FAM) prepared in Example 6-2 in HCT116 cells by flow cytometry.

FIG. 24 shows a result of treating HeLa cells with a G2 Janus-type dendritic polyelectrolyte zwitterion (Example 6-2) into which a fluorescent label is introduced and analyzing the same by confocal microscopy (LSM 980, Carl Zeiss, Germany).

FIG. 25 shows a result of analyzing the cytotoxicity of G2 Janus-type dendritic polyelectrolyte zwitterions ((+M)₂(−M)₂ conjugate_FAM) into which a fluorescentlabel is introduced, prepared in Examples 6-1 and 6-2, and G2 Janus-type dendrimers into which the fluorescent label is introduced, prepared in Comparative Examples 2-1 and 2-2, by flow cytometry (FACS).

FIG. 26 shows the MALDI-TOF spectrum of a G2 Janus-type dendritic polyelectrolyte zwitterion prepared in Example 7 ((+6)₂(−6)₂ conjugate 2).

FIG. 27 shows the MALDI-TOF spectrum of a G2 Janus-type dendritic polyelectrolyte zwitterion prepared in Example 8 ((+6)₂(−6)₂ conjugate_3).

FIG. 28 shows a result of treating HeLa cells with a complex of a Janus-type dendritic polyelectrolyte zwitterion (+6)₂(−6)₂ conjugate_2 prepared in Example 7 and EGFP (enhanced green fluorescent protein) mRNA and quantifying the expressed GFP protein by flow cytometry.

FIG. 29 shows a result of treating HeLa cells with a complex of a Janus-type dendritic polyelectrolyte zwitterion (+6)₂(−6)₂ conjugate 3 prepared in Example 8 and EGFP (enhanced green fluorescent protein) mRNA and quantifying the expressed GFP protein by flow cytometry.

FIG. 30 shows a result of quantifying the expression of a complex ((+6)₂(−6)₂_conjugate 3_complex) carrying EGFP mRNA in vivo.

### BEST MODE

Prior to the disclosure and description of the compounds, compositions and/or methods of the present disclosure, it should be understood that the aspects described below are not limited to specific particular compounds, synthetic methods or uses, which may differ. Additionally, it should be understood that the terminology used herein is for the purpose of describing particular aspects only and is not intended to limit the present disclosure.

In this specification and the claims below, reference will be made to a number of terms which are hereby defined to have the following meanings.

Unless specifically stated otherwise, wt % of an ingredient is based on the total weight of a formulation or a composition in which the ingredient is included.

In the present disclosure, “A and/or B” means “A and B, or A or B”.

In the present disclosure, a “dendrimer” refers to a molecule having a regular branched structure, and it is generally synthesized by connecting constituent units one by one. Dendrimers can be used for various purposes such as drug carriers, therapeutic agents, bioimaging, contrast agents, etc. depending on their structure and characteristics.

In the present disclosure, “Janus” is named after the two-faced god of Roman mythology, and refers to a particle containing two structures in one molecule. Narrowly, it refers to a spherical particle having a different structure in each half. But, in the present disclosure, it is used as a general meaning that includes a dendrimer molecule having two parts to which materials with different properties are bound.

In the present disclosure, the “amino acid” is used in the broadest sense and is intended to include naturally occurring L-amino acids or residues. One-letter abbreviations and/or three-letter abbreviations commonly used for naturally occurring amino acids may be used in the present specification. The amino acid includes D-amino acids as well as chemically modified amino acids such as amino acid analogues, naturally occurring amino acids that are not normally incorporated into proteins, e.g. norleucine, and chemically synthesized compounds having characteristics known in the art, including carboxy- and/or amino-terminal amino acids. For example, analogues or mimetics of phenylalanine or proline that allow for conformational restrictions of peptide compounds identical to natural Phe or Pro are included within the definition of the amino acid. Such analogues and mimetics are referred to herein as “functional equivalents” of amino acids. Other examples of the amino acid are described in the literature [Roberts and Vellaccio, The Peptides: Analysis, Synthesis, Biology, Eds. Gross and Meiehofer, Vol. 5, p. 341 (Academic Press, Inc.: N.Y. 1983)].

In the present disclosure, the “peptide” includes all of proteins, protein fragments and peptides isolated from naturally existing ones, or synthesized chemically or by recombinant techniques.

In the present disclosure, the peptide may include those that are 70% or higher, specifically 90% or higher, more specifically 95% or higher, identical to the original “peptide”.

In the present disclosure, “identical” means that there is no significant change in characteristics such as the secondary structure, hydropathic nature, etc. of the polypeptide even when one amino acid is replaced with another amino acid. Variations in amino acids may be achieved based on the relative similarity of amino acid side chain residues, such as polarity, charge, solubility, hydrophobicity, hydrophilicity, and/or amphiphilic nature.

For example, amino acids may be classified as i) hydrophobic (norleucine, methionine, alanine, valine, leucine or isoleucine), ii) neutral and hydrophilic (cysteine, serine, threonine, asparagine or glutamine), iii) acidic (aspartic acid or glutamic acid), iv) basic (histidine, lysine or arginine), v) affecting chain direction (glycine or proline), and vi) aromatic (tryptophan, tyrosine or phenylalanine), depending on the properties of common side chains. A conservative substitution will involve exchange of a member of one of each of these classes for another member of the same class.

Upon analysis of the size, shape and type of amino acid side chain residues, it can be seen that arginine, lysine and histidine are positively charged residues; aspartic acid and glutamic acid are negatively charged residues; alanine, glycine and serine have similar sizes; and phenylalanine, tryptophan and tyrosine have similar shapes. Therefore, based on these considerations, arginine, lysine and histidine; aspartic acid, glutamic acid; alanine, glycine and serine; and phenylalanine, tryptophan and tyrosine can be said to be biologically equivalent in function.

Hereinafter, the present disclosure is described in detail.

An aspect of the present disclosure relates to a polyelectrolyte-zwitterion-based dendritic compound represented by the following General Formula 1 or 2.

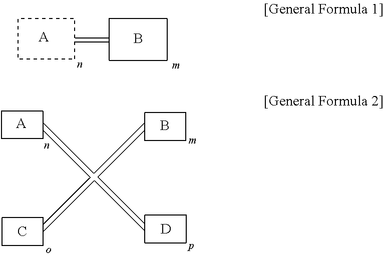

In the above formulas, each of A and C may independently be at least one selected from a group consisting of arginine (Arg, R), histidine (His, H), lysine (Lys, K), ornithine (Orn) and guanidine,
each of B and D is independently at least one selected from a group consisting of glutamic acid (Glu, E) and aspartic acid (Asp, D), and
each of n, m, o and p may independently be an integer selected from 3 to 16, specifically from 3 to 12, more specifically from 3 to 6.

Each of the arginine (Arg, R), histidine (His, H), lysine (Lys, K), ornithine (Orn), guanidine, glutamic acid (Glu, E) and aspartic acid (Asp, D) may independently be an L- or D-amino acid.

Specifically, the dendritic compound may be one in which n, m, o and p are all the same under tsome conditions between of pH 2 and pH 13.

In General Formula 1, the repeating unit represented by A and the repeating unit represented by B may be connected to each other via a linker and, in General Formula 2, the repeating unit represented by A, the repeating unit represented by B, the repeating unit represented by C and the repeating unit represented by D may be connected to each other via a linker.

The linker may be at least one or more lysine residue (Lys or K). Since the lysine has a side chain reactive group, it can provide a reactive group for polymerization of the repeating units represented by A, B, C and D.

Specifically, the dendritic polyelectrolyte-zwitterion(DPZ) compound represented by General Formula 1 or 2 may be any one selected from those represented by Chemical Formulas 1-1 to 1-6.

Another aspect of the present disclosure relates to a dendritic polyelectrolyte zwitterion, which contains a 1st to 5th generation dendrimer-based core; and a positively charged peptide represented by SEQ ID NO: 1 and/or a negatively charged peptide represented by SEQ ID NO: 2 bonded to the terminal functional group of the core.

[SEQ ID NO: 1]
[X₁]ₙ
[SEQ ID NO: 2]
[X₂]ₙ

In the above sequences, X₁ is any one selected from arginine (Arg, R), histidine (His, H), lysine (Lys, K), ornithine (Orn) and guanidine, and X₂ is glutamic acid (Glu, E) or aspartic acid (Asp, D), and
n may be an integer from 3 to 16, specifically from 3 to 12, more specifically from 3 to 6.

In the present disclosure, the “core” refers to a material that binds to a positively charged peptide and a negatively charged peptide and serves as a support that allows the positively charged peptide and the negatively charged peptide to be arranged at regular intervals.

The dendrimer-based core may be, for example, any one selected from a group consisting of a polyamidoamine (PAMAM) dendrimer, a polylysine dendrimer, a polyimine (PI) dendrimer, a polypropyleneimine (PPI) dendrimer, a polyester dendrimer, a polyamide dendrimer, a polyurethane dendrimer, a polyornithine dendrimer, a carbosilane dendrimer, a polyether dendrimer, a polyglutamic acid dendrimer, a polyaspartic acid dendrimer, a polyglycerol dendrimer and a polymelamine dendrimer, although it is not particularly limited as long as it contains a terminal functional group capable of binding to a positively charged peptide and/or a negatively charged peptide.

The dendrimer-based core is specifically of the 1st to 5th generation, more specifically of the 1st or 2nd generation.

In the present disclosure, the generation of the “dendrimer” can be specified depending on the level of overlap of the base compound forming the dendrimer. A dendrimer can be expressed as a 1st generation dendrimer (G1), a 2nd generation dendrimer (G2), a 3rd generation dendrimer (G3), a 4th generation dendrimer (G4) or a 5th generation dendrimer (G5). The number of the generation increases one by one as one additional base compound is added.

When the dendrimer-based core is a polylysine dendrimer, the polylysine dendrimer is composed of at least one or more “lysine (K)”, and one additional lysine is bonded to the lysine core, forming a 1st generation dendrimer. A 1st generation dendrimer has one or more “branches” attached to the core. A 2nd generation dendrimer can be formed by reacting lysine with a 1st generation dendrimer. 3rd, 4th, 5th generations, etc. can be formed through similar reactions.

The polylysine dendrimer consisting of repeating lysines is not particularly limited as long as it is of the 1st generation or higher. Specifically, it may be of the 1st to 5th generation, more specifically of the 1st or 2nd generation. The polylysine dendrimer can be produced through subsequent coupling/deprotection using lysine, and branches can be generated because lysine has three reactive groups.

Specifically, the lysine has side chains of an amino group, a carboxyl group and an amino group, and has a left-handed α-hydrogen stereostructure. That is, the core consisting of one or more lysine residues binds to one or more lysine residues, and the binding may be through a peptide bond with the side chain of the lysine residue.

In the present disclosure, the polylysine has a plurality of terminal amino groups on the surface. A plurality of positively charged peptides represented by SEQ ID NO: 1 and/or negatively charged peptides represented by SEQ ID NO: 2 may be bonded thereto and arranged to be spaced apart from each other.

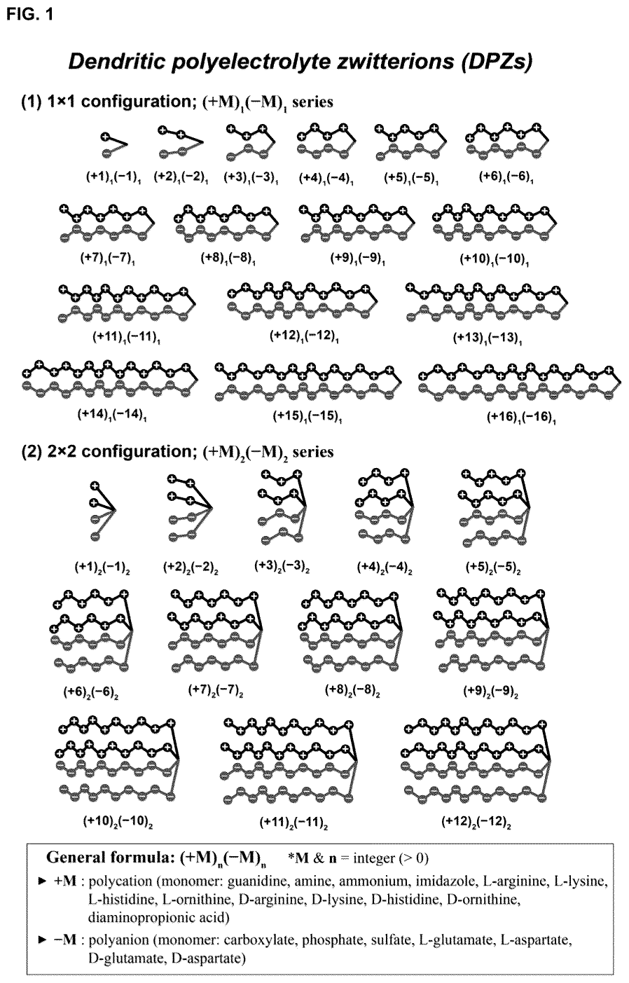

Specifically, each of the positively charged peptide represented by SEQ ID NO: 1 and the negatively charged peptide represented by SEQ ID NO: 2 may be bonded to the terminal amino group of the polylysine dendrimer. That is to say, the dendritic polyelectrolyte zwitterion according to the present disclosure can exhibit polyelectrolyte properties and zwitterion properties at the same time as the peptides with different charges are bonded alternately to the terminal amino group of the dendrimer (FIG. 1).

The dendritic polyelectrolyte zwitterion according to the present disclosure is characterized in that the same number of positively charged peptides represented by SEQ ID NO: 1 and negatively charged peptides represented by SEQ ID NO: 2 are bonded. If the number of one of the peptides is larger or smaller than that of the other peptide, the zwitterion characteristics cannot be exhibited properly as the total charge (net charge) deviates from 0 and, as a result, cytotoxicity may occur. For example, when the number of positively charged peptide chains exceeds the number of negatively charged peptide chains, the dendrimer molecule becomes positively charged, causing cytotoxicity. And, when the number of negatively charged peptide chains exceeds that of the positively charged peptide chains, there is no cytotoxicity, but the intracellular delivery efficiency may decrease significantly.

The number of positively charged peptides and negatively charged peptides per polylysine dendrimer molecule is not particularly limited, but the optimal number may be selected depending on the terminal amino group present on the surface of the polylysine dendrimer. For example, when the polylysine dendrimer is a 1st generation polylysine dendrimer, one strand of the positively charged peptide chain represented by SEQ ID NO: 1 and one strand of the negatively charged peptide chain represented by SEQ ID NO: 2 may be bonded. In addition, when the polylysine dendrimer is a 2nd generation polylysine dendrimer, two strands of the positively charged peptide chain represented by SEQ ID NO: 1 and two strands of the negatively charged peptide chain represented by SEQ ID NO: 2 may be bonded.

In the above sequence, each of arginine (Arg, R), histidine (His, H), lysine (Lys, K), ornithine (Orn), guanidine, glutamic acid (Glu, E) and aspartic acid (Asp, D) may independently be an L- or D-amino acid.

The positively charged peptide is not particularly limited as long as it consists of positively charged amino acid residues, but may be represented by any one selected from a group consisting of SEQ ID NOS: 3 to 98, specifically from a group consisting of SEQ ID NOS: 3 to 50, more specifically from a group consisting of SEQ ID NOS: 3 to 8.

The negatively charged peptide is not particularly limited as long as it consists of a negatively charged amino acid residue, but may be represented by any one selected from a group consisting of SEQ ID NOS: 99 to 162, specifically from a group consisting of SEQ ID NOS: 99 to 130.

The dendritic polyelectrolyte zwitterion according to the present disclosure may be represented by any one of Chemical Formulas 1-1 to 1-6. The weight-average molecular weight (Mw) of the dendritic polyelectrolyte zwitterion according to the present disclosure may be 2,200 to 4,000 Da. It is a safe material that does not exhibit toxicity to cells even at high concentrations.

The dendritic polyelectrolyte zwitterion according to the present disclosure can deliver a drug and can be labeled with a fluorescent material. It is possible to prepare a dendritic polyelectrolyte zwitterion that can deliver a drug or a contrast agent to a target through combination with the drug or contrast agent. For example, it may be represented by any one of Chemical Formulas 2-1 to 2-6.

In the present disclosure, the “drug” may be a synthetic compound drug for alleviating, preventing, treating or diagnosing a disease, wound or specific symptom. Specifically, it may be any one selected from doxorubicin, daunorubicin, epirubicin, idarubicin, valrubicin, mitoxantrone, dexamethasone, triamcinolone, beclomethasone dipropionate, triamcinolone acetonide, triamcinolone diacetate, betamethasone dipropionate, testosterone, budesonide, 17α-ethinylestradiol, levonorgestrel, fluticasone propionate, sorafenib, paclitaxel, docetaxel, doxorubicin, cyclosporine A, amphotericin B, indinavir, rapamycin, doxorubicin, coenzyme Q₁₀, ursodeoxycholic acid, ilaprazole, imatinib mesylate and tanespimycin.

In the present disclosure, the “contrast agent” refers to any material used to contrast structures or fluids in vivo in medical imaging. The contrast agent may include a radiopaque contrast agent, a paramagnetic contrast agent, a superparamagnetic contrast agent, a computed tomography (CT) contrast agent or other contrast agents, although not being limited thereto. For example, the radiopaque contrast agent (for X-ray imaging, etc.) may include inorganic iodine compounds, organic iodine compounds (e.g., diatrizoate), radiopaque metals (e.g., silver, gold, platinum, etc.) and their salts, and other radiopaque compounds (e.g., calcium salts, barium salts such as barium sulfate, tantalum, and tantalum oxide). The paramagnetic contrast agent (for MR imaging) may include gadolinium diethylenetriaminepentaacetic acid (Gd-DTPA) and its derivatives, as well as other gadolinium, manganese, iron, dysprosium, copper, europium, erbium, chromium, nickel and cobalt complexes, such as 1,4,7,10-tetraazacyclododecane-N,N′,N″,N′″-tetraacetic acid (DOTA), ethylenediaminetetraacetic acid (EDTA), 1,4,7,10-tetraazacyclododecane-N,-N′,N″-triacetic acid (D03A), 1,4,7-triazacyclononane-N,N′,N″-triacetic acid (NOTA), 1,4,8,10-tetraazacyclotetradecane-N,N′,N″,N′″-tetraacetic acid (TETA), and hydroxybenzylethylene-diaminediacetic acid (HBED). The superparamagnetic contrast agent (for MR imaging) may include magnetite, superparamagnetic iron oxide (SPIO), ultrasmall superparamagnetic iron oxide (USPIO), and monocrystalline iron oxide. Other suitable contrast agents may include iodinated and non-iodinated, ionic and non-ionic CT contrast agents, contrast agents such as spin labels, or other diagnostically effective agents. Additionally, the contrast agent may include β-galactosidase, green fluorescent protein, blue fluorescent protein, or luciferase. When expressed in cells, it may include a marker gene encoding an easily detectable protein. Various labels such as radionuclides, fluorescent materials, enzymes, enzyme substrates, enzyme cofactors, enzyme inhibitors, etc. can be used.

The fluorescent material may be one or more selected from a group consisting of fluorescein, carboxyfluorescein (FAM), rhodamine, Texas Red, tetramethylrhodamine, carboxyrhodamine, carboxyrhodamine 6G, carboxyrhodol, carboxyrhodamine 110, cascade blue, cascade yellow, coumarin, Cy2, Cy3, Cy3.5, Cy5, Cy5.5, Cy-chrome, phycoerythrin, PerCP (peridinin-chlorophyll α-protein), PerCP-Cy5.5, JOE (6-carboxy-4′,5′-dichloro-2′,7′-dimethoxyfluorescein), NED, ROX (5-(and 6)-carboxy-X-rhodamine), HEX, Lucifer yellow, Marina Blue, Oregon Green 488, Oregon Green 500, Oregon Green 514, Alexa Fluor (trade name) 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, 7-amino-4-methylcoumarin-3-acetic acid, BODIPY (brand name) FL, BODIPY FL-Br2, BODIPY 530/550, BODIPY 558/568, BODIPY 564/570, BODIPY 576/589, BODIPY 581/591, BODIPY 630/650, BODIPY 650/665, BODIPY R6G, BODIPY TMR, and BODIPY TR, although not being limited thereto.

The drug and contrast agent may be additionally bound to the functional group at the other end to which the peptide is not bound, based on the core of the dendrimer.

According to a specific exemplary embodiment of the present disclosure, a composition containing DPZ can be used as a composition that can superiorly deliver a contrast agent or a drug into cells.

Another aspect of the present disclosure relates to a dendritic polyelectrolyte zwitterion, which contains a 1st to 5th generation dendrimer-based core; and a positively charged peptide represented by SEQ ID NO: 1 and/or a negatively charged peptide represented by SEQ ID NO: 2 bound to one terminal functional group based on the dendrimer core, wherein a hydrophobic compound is bound to the other terminal functional group based on the dendrimer core.

[SEQ ID NO: 1]
[X₁]ₙ
[SEQ ID NO: 2]
[X₂]ₙ

In the above sequences, X₁ is any one selected from arginine (Arg, R), histidine (His, H), lysine (Lys, K), ornithine (Orn) and guanidine, and X₂ is glutamic acid (Glu, E) or aspartic acid (Asp, D), and n is an integer from 3 to 16.

The dendrimer-based core is the same as the dendrimer-based core described above with regard to the dendritic polyelectrolyte zwitterion. In the present disclosure, the dendrimer-based core is characterized in that a peptide or hydrophobic compound is bound to the terminal functional group at each end.

The dendrimer-based core has terminal functional groups on the surface according to the generation number. Specifically, the number of the terminal functional groups is equal to 2^{x}, where x is the generation number of the dendrimer. For example, a 1st generation dendrimer-based core has two surface terminal functional groups, a 2nd generation has four surface terminal functional groups, a 3rd generation has eight surface terminal functional groups, a 4th generation has sixteen surface terminal functional groups, and a 5th generation has thirty two surface terminal functional groups.

Among the total terminal functional groups (2^{x}) present in the dendrimer-based core, half (50%) may be bound to the peptide (one side) and the other half (50%) may be bound independently to the hydrophobic compound or the amino acid derivative (the other side).

When the dendrimer-based core is lysine, it may be specifically represented by [Chemical Formula A] or [Chemical Formula B] below, and the terminal functional group may be the N-terminal or the side chain terminal of lysine, or both.

Lys(α,ε) [Chemical Formula A]G1

Lys(α,ε)-Lys [Chemical Formula B]G2

Lys(α,ε)

The Janus-type dendritic polyelectrolyte zwitterion of the present disclosure is one in which materials with different characteristics are bonded to one side and the other side of the total terminal functional groups based on the dendrimer-based core molecule. The dendritic polyelectrolyte zwitterion is formed as each of positively charged and negatively charged hydrophilic peptides is bonded to the amino group at the a position of lysine and the amino group of the side chain at the F position, and a hydrophobic compound is bonded to the other side.

The same number of positively charged peptides represented by SEQ ID NO: 1 and negatively charged peptides represented by SEQ ID NO: 2 may be bound to the ends of the dendrimer-based core. For example, when the number of positively charged peptide chains exceeds the number of negatively charged peptide chains, the dendrimer molecule becomes positively charged, causing cytotoxicity. And, when the number of negatively charged peptide chains exceeds that of the positively charged peptide chains, there is no cytotoxicity, but the intracellular delivery efficiency may decrease significantly.

The number of positively charged peptides and negatively charged peptides per dendrimer-based core molecule is not particularly limited, but the optimal number may be selected depending on the terminal functional groups present on the surface of the dendrimer-based core. For example, when the dendrimer-based core is of the 2nd generation, one strand of the positively charged peptide chain represented by SEQ ID NO: 1 and one strand of the negatively charged peptide chain represented by SEQ ID NO: 2 may be bound. In addition, when the dendrimer-based core is of the 3rd generation, two strands of the positively charged peptide chain represented by SEQ ID NO: 1 and two strands of the negatively charged peptide chain represented by SEQ ID NO: 2 may be combined.

The positively charged peptide is not particularly limited as long as it consists of positively charged amino acid residues, but may be represented by any one selected from a group consisting of SEQ ID NOS: 3 to 98, specifically from a group consisting of SEQ ID NOS: 3 to 50, more specifically from a group consisting of SEQ ID NOS: 3 to 8.

The negatively charged peptide is not particularly limited as long as it consists of a negatively charged amino acid residue, but may be represented by any one selected from a group consisting of SEQ ID NOS: 99 to 162, most specifically SEQ ID NOS: 99 to 130.

Because of the Janus form as described above, the dendritic polyelectrolyte zwitterion self-assembles into a nanostructure with an elongated fiber-shaped nanofiber structure in solution. It is formed since the Janus-type polyelectrolyte-zwitterion-based dendrimer has a polyelectrolyte-zwitterion molecular structure and an amphiphilic property due to the presence of both hydrophilic and hydrophobic groups. It is a thermodynamically stable and uniform structure formed by the amphiphilic material.

Due to the amphiphilic nature of this nanofiber form, the Janus form allows the dendritic polyelectrolyte zwitterion to be protected from various mechanisms in the human body and move efficiently to the desired site.

In addition, since the Janus-type dendritic polyelectrolyte zwitterion contains a hydrophobic compound bound to the dendrimer molecule, it can be stably delivered into cells without toxicity to the cells. In addition, when administered in vivo, it has non-fouling property for biological substances (nucleic acids, proteins, etc.) present in the body. So, the Janus-type dendritic polyelectrolyte zwitterion can solve side effects such as additional degradation of biological functions. Therefore, the Janus-type dendritic polyelectrolyte zwitterion can be excellently used for various purposes such as drug carriers, therapeutic agents, bioimaging, and contrast agents.

The hydrophobic compound is not particularly limited as long as it is a hydrophobic material. For example, the hydrophobic compound may be one or more selected from a group consisting of fatty acids, bile acids, hydrophobic drugs, contrast agents, and fluorescent materials.

The fatty acid is a hydrophobic fatty acid capable of amide bonding with the N-terminal of a polylysine dendrimer through a carboxyl group, and includes C₃-C₃₀ fatty acids and aliphatic carboxylic acids, e.g., C₄-C₂₀ aliphatic carboxylic acids. For example, the aliphatic carboxylic acids include saturated aliphatic carboxylic acids and unsaturated aliphatic carboxylic acids, which may be linear or branched aliphatic carboxylic acids.

The saturated aliphatic carboxylic acids may include one or more saturated aliphatic monocarboxylic acid selected from a group consisting of butanoic acid (butyric acid), pentanoic acid (valeric acid), hexanoic acid (caproic acid), heptanoic acid (enanthic acid), octanoic acid (caprylic acid), nonanoic acid (pelargonic acid), decanoic acid (capric acid), undecanoic acid (undecylic acid), dodecanoic acid (lauric acid), tridecanoic acid (tridecylic acid), tetradecanoic acid (myristic acid), pentadecanoic acid (pentadecylic acid), hexadecanoic acid (palmitic acid), heptadecanoic acid (margaric acid), octadecanoic acid (stearic acid), nonadecanoic acid (nonadecylic acid), icosanoic acid (arachidic acid), heneicosanoic acid (heneicosylic acid), docosanoic acid (behenic acid), tricosanoic acid (tricosylic acid), tetracosanoic acid (lignoceric acid), pentocosanoic acid (pentacosylic acid), hexacosanoic acid (cerotic acid), heptocosanoic acid (heptacosylic acid), octacosanoic acid (montanic acid), nonacosanoic acid (nonacosylic acid) and triacontanoic acid (melissic acid), although not being limited thereto.

In addition, aliphatic dicarboxylic acids such as succinic acid, glutaric acid, adipic acid, pimelic acid, suberic acid, azelaic acid and sebacic acid; and aliphatic tricarboxylic acids such as citric acid, isocitric acid and propane-1,2,3-tricarboxylic acid may be included.

In addition, the unsaturated aliphatic carboxylic acid may be any one or more selected from a group consisting of butenoic acid such as crotonic acid, pentenoic acid, hexenoic acid, heptenoic acid, octenoic acid, nonenoic acid, decenoic acid such as cis-4-decenoic acid (obtusilic acid; 10:1(n-6)), cis-9-decenoic acid (caproleic acid; (10:(n-1)), aconitic acid, myristoleic acid (14: 1), α-linolenic acid (18:3), stearidonic acid (18:4), eicosapentaenoic acid (EPA; 20:5), docosahexaenoic acid (22:6), linoleic acid (18:2), γ-linolenic acid (18:3), dihomo-γ-linolenic acid (20: 3), arachidonic acid (20:4), adrenic acid (22:4), palmitoleic acid (16:1), vaccenic acid (18:1), paullinic acid (20:1), oleic acid (18:1), elaidic acid (trans-18:1), gondoic acid (11-eicosenoic acid; 20:1), erucic acid (22:1), nervonic acid (24:1), mead acid (20:3) and ximenic acid (26:1).

For example, the hydrophobic fatty acid may be one or more selected from a group consisting of lauric acid (dodecanoic acid), tridecylic acid (tridecanoic acid), myristic acid (tetradecanoic acid), pentadecylic acid (pentadecanoic acid), palmitic acid (hexadecanoic acid), margaric acid (heptadecanoic acid), stearic acid (octadecanoic acid), nonadecylic acid (nonadecanoic acid), arachidic acid (icosanoic acid, heneicosylic acid (heneicosanoic acid), behenic acid (docosanoic acid), myristoleic acid (14:1), α-linolenic acid (18:3), stearidonic acid (18:4), eicosapentaenoic acid (EPA; 20:5), docosahexaenoic acid (DHA; 22:6), linoleic acid (18:2), γ-linolenic acid (18:3), dihomo-γ-linolenic acid (20:3), arachidonic acid (20:4), adrenic acid (22:4)), palmitoleic acid (16:1), vaccenic acid (18:1), paullinic acid (20:1), oleic acid (18:1), elaidic acid (trans-18:1), gondoic acid (11-eicosenoic acid; 20:1) and erucic acid (22:1), more specifically, one or more selected from a group consisting of dodecanoic acid, myristic acid, palmitic acid, stearic acid, arachidic acid, behenic acid, palmitoleic acid and oleic acid.

The hydrophobic drug is not particularly limited thereto, but may specifically include a chemical substance or a biodrug having a water solubility of about 10 mg/mL or lower. For example, the hydrophobic drug may be an anthracycline-based substance, a hydrophobic glucocorticoid, a steroid-based substance, a taxane-based drug, a cyclic peptide-based drug, or a combination thereof. The anthracycline-based substance may be doxorubicin, daunorubicin, epirubicin, idarubicin, valrubicin, mitoxantrone, or a combination thereof. The hydrophobic glucocorticoid may be, for example, dexamethasone, triamcinolone, beclomethasone dipropionate, triamcinolone acetonide, triamcinolone diacetate, betamethasone dipropionate, testosterone, budesonide, 17α-ethinylestradiol, levonorgestrel, fluticasone propionate, or a combination thereof. For example, the hydrophobic drug may be sorafenib, paclitaxel, docetaxel, doxorubicin, cyclosporine A, amphotericin B, indinavir, rapamycin, doxorubicin, coenzyme Q₁₀, ursodeoxycholic acid, ilaprazole, imatinib mesylate, or a combination thereof.

The contrast agent (imaging agent or contrast medium) refers to a substance which increases the contrast of the image of a tissue or a blood vessel by artificially increasing the difference in X-ray absorption during magnetic resonance imaging or computed tomography. The contrast agent may be, for example, a transition element or a chelate complex of a transition element.

The fluorescent material is not particularly limited as long as it exhibits hydrophobicity, but specifically may be one or more selected from a group consisting of fluorescein, carboxyfluorescein (FAM), rhodamine, Texas Red, tetramethylrhodamine, carboxyrhodamine, carboxyrhodamine 6G, carboxyrodol, carboxyrhodamine 110, Cascade Blue, Cascade Yellow, coumarin, Cy2, Cy3, Cy3.5, Cy5, Cy5.5, Cy-chromium, phycoerythrin, PerCP (peridinine-chlorophyll α-protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer yellow, Marina Blue, Oregon Green 488, Oregon Green 500, Oregon Green Green) 514, Alexa Fluor (brand name) 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, 7-amino-4-methylcoumarin-3-acetic acid, BODIPY (brand name) FL, BODIPY FL-Br2, BODIPY 530/550, BODIPY 558/568, BODIPY 564/570, BODIPY 576/589, BODIPY 581/591, BODIPY 630/650, BODIPY 650/665, BODIPY R6G, BODIPY TMR and BODIPY TR, although not being limited thereto.

The amino acid derivative may be one or more selected from a group consisting of allantoin, N,N-dimethylglycine, homoserine, hypoxanthine, lactate, malic acid and glycerol 3-phosphate.

The Janus-type dendritic polyelectrolyte zwitterion according to the present disclosure may be represented by any one of Chemical Formulas 3-1 to 3-6, 4-1 to 4-12, 5-1 to 5-4, 6-1 to 6-2, 7 and 8. In the above Chemical Formulas, a hydrophobic compound or an amino acid derivative may be represented by one or more of R₁ to R₄, and each of R₁ to R₄ may independently be selected from the above-mentioned hydrophobic compounds or amino acid derivatives.

The structure that has polyelectrolyte and zwitterion properties as well as hydrophilic and hydrophobic properties in the Janus-type dendritic polyelectrolyte zwitterion according to the present disclosure is a newly designed structure that does not originate from anywhere. Most conventional drug carriers have polyelectrolyte properties. It is known that drug carriers having polyelectrolyte properties can form a strong bond with the negatively charged cell membrane and can be effectively delivered into cells. However, positively charged particles induce high toxicity and immune responses in vivo, and there is a problem of the polyelectrolyte effect where nanoparticles agglomerate with each other at ion concentrations similar to in vivo conditions. In fact, although various positively charged nanoparticles have been designed to suppress aggregation through surface modification, but in this case, the intracellular delivery efficiency is reduced, or even when delivered, they do not remain bound to other substances (nucleic acids, proteins, etc.) in vivo but remain in an aggregated state. This may cause side effects that impede biological functions.

However, the present disclosure allows the positively charged peptide represented by SEQ ID NO: 1 and/or the negatively charged peptide represented by SEQ ID NO: 2 to be alternately bonded to one terminal functional group based on the dendrimer core (specifically polylysine), thereby providing stable polyelectrolyte and zwitterionic properties together with appropriate hydrophilicity for self-assembly, and can achieve high intracellular delivery efficiency, low toxicity, and high structural stability at physiological salt concentrations even when a hydrophobic compound is bound to the other functional group.

According to a specific exemplary embodiment of the present disclosure, the Janus-type dendritic polyelectrolyte zwitterion of the present disclosure does not aggregate with each other, but self-assembles into a nanofiber-like structure, which exists stably in solution. In addition, it has the advantage of having non-fouling property for biomaterials (nucleic acids, proteins, etc.).

In addition, since the Janus-type dendritic polyelectrolyte zwitterion according to the present disclosure does not form cross-linkages or aggregates even when mixed with external substances such as nucleic acid materials, proteins, etc., it can be advantageously used immediately without additional surface modification or addition of a carrier. Therefore, it can be used in various fields such as medicine, stimulus-responsive molecular machines, cosmetics, etc.

Another aspect of the present disclosure relates to a nanostructure including the Janus-type dendritic polyelectrolyte zwitterion. It can have numerous medical uses. Specifically, it can be used to deliver a drug, a fluorescent material, etc. to a subject in need of treatment or to deliver a target gene or a drug to cells and tissues.

Another aspect of the present disclosure relates to a gene or drug carrier containing: the Janus-type dendritic polyelectrolyte zwitterion described above; and a target gene or a drug loaded in the dendritic polyelectrolyte zwitterion.

As described above, since the Janus-type dendritic polyelectrolyte zwitterion according to the present disclosure is an amphiphilic molecule that has both hydrophilic and hydrophobic properties, and therefore has the characteristics of an amphiphilic molecule, it can form a nanostructure with a specific structure through self-assembly in solution.

The Janus-type dendritic polyelectrolyte zwitterion of the present disclosure forms a supramolecular nanostructure with an elongated fiber structure in the form of nanofibers in solution. In the nanofiber structure, amphiphilic molecules form an aggregated nanostructure with uniform size and shape. In the present disclosure, the nanofiber structure may be an elongated fiber having a thread structure.

The nanostructure may typically have an average diameter of 10 to 400 nm, specifically 8 to 30 nm.

The Janus-type dendritic polyelectrolyte zwitterion may be loaded with a target gene or a drug. Specifically, when the Janus-type dendritic polyelectrolyte zwitterion and the target gene or drug are mixed in solution, the target gene or drug is encapsulated in a nanostructure formed by self-assembly of the Janus-type dendritic polyelectrolyte zwitterion. Since it exists very stably in solution and has superior non-fouling property, it can not only provide long-term protection from substances present outside and inside the body, but can also be efficiently delivered into cells. In addition, the above-described process is advantageous in that it proceeds easily and fast automatically through self-assembly in solution. In addition, the nanostructure present in the composition of the present disclosure has the advantage of having non-fouling property for external substances present inside and outside the living body. So, it can be stored and used without separate surface modification or a carrier.

The nanostructure of the present disclosure improves the problems of conventional drug carriers, i.e., the inability to maintain shape for a long period of time due to unstable membrane structure of the drug carrier or rapidly decreased concentration in vivo due to binding to external substances in the body. In addition, the nanostructure according to the present disclosure can be stored in various solvents such as deionized water (DW), PBS, etc. And, stability is maintained during storage since the average diameter and size distribution remain unchanged as compared to conventional drug carriers. Furthermore, it was confirmed that the nanostructure of the present disclosure can be significantly delivered into cells (into the nuclei and cytoplasm of cells) and, in particular, can efficiently deliver the loaded target gene or drug for expression as a protein.

The nanostructure according to the present disclosure can be administered to a subject using techniques known in the art. It will be appreciated that the actually desired amount of nanostructure for a particular patient will vary depending on the particular compound used, the particular composition formulated, the mode of application, and the particular site and target being treated.

The nanostructure according to the present disclosure is effective in delivering a target gene or a drug into cells, and can ultimately be used for the treatment or prevention of many diseases, including genetic diseases and cancer. As used herein, the term “treat” is defined as reducing the symptoms of a disease or maintaining the symptoms of a disease so that they do not worsen. The term “treat” is also defined as preventing any symptoms associated with a particular disease.

The target gene or drug is a substance to be delivered into cells, and is not particularly limited as long as it does not impair the purpose of the present disclosure. The gene may be a plasmid, an mRNA, an RNA, a DNA, or a combination thereof, and the drug may be a low-molecular-weight drug, a gene drug, a protein drug, an antibody drug, a synthetic compound drug, or a combination thereof.

The gene may be a normal target gene related to a disease or a gene that suppresses the expression of a target protein. For example, it may be a cancer-treating gene that induces death of cancer cells and ultimately degenerates tumor, such as a tumor suppressor gene, an immunoregulatory gene, a cytokine gene, a chemokine gene, an antigenic gene, a suicide gene, a cytotoxic gene, a cytostatic gene, a pro-apoptotic gene, an anti-angiogenic gene, etc., although not being limited thereto. In addition, many therapeutic genes that can be usefully used to treat various diseases can also be delivered by the complex according to a specific exemplary embodiment. For example, genes encoding cytokines, interleukins, chemokines or colony-stimulating factors, genes expressing tissue-type plasminogen activator (tPA) or urokinase, and LAL-producing genes, which provides a sustained thrombotic effect and prevents hypercholesterolemia, may be included, and various polynucleotides for treating viral, malignant and inflammatory diseases and conditions such as cystic fibrosis, adenosine deaminase deficiency, and AIDS may also be included. The base sequence of the gene or polynucleotide can be obtained from a base sequence database such as GenBank or EMBL.

The cancer may be ovarian cancer, colorectal cancer, pancreatic cancer, stomach cancer, liver cancer, breast cancer, cervical cancer, thyroid cancer, parathyroid cancer, lung cancer, non-small-cell lung cancer, prostate cancer, gallbladder cancer, biliary tract cancer, non-Hodgkin lymphoma, Hodgkin lymphoma, blood cancer, bladder cancer, kidney cancer, melanoma, colon cancer, bone cancer, skin cancer, head cancer, uterine cancer, rectal cancer, brain cancer, perianal cancer, fallopian tube carcinoma, endometrial carcinoma, vaginal cancer, vulvar carcinoma, esophageal cancer, small intestine cancer, endocrine cancer, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma, although not being limited thereto.

The composition of the present disclosure can be administered orally or parenterally (e.g., intravenously, subcutaneously, intraperitoneally, intranasally, by inhalation, or by topical application) depending on the desired method, and the dosage varies depending on the patient's condition and weight, the degree of a disease, drug type, and administration route and time, but can be selected appropriately by a person skilled in the art.

The composition of the present disclosure is administered in a pharmaceutically effective amount. In the present disclosure, the “pharmaceutically effective amount” means an amount sufficient to treat or diagnose a disease with a reasonable benefit/risk ratio applicable to medical treatment or diagnosis, and the effective dose level may be determined based on factors well known in the medical field, such as the patient's disease type, severity, drug activity, sensitivity to the drug, time of administration, route of administration, excretion rate, duration of treatment, concurrently used drugs, and other factors. The pharmaceutical composition according to the present disclosure may be administered as an individual therapeutic agent or in combination with another therapeutic agent. It may be administered sequentially or simultaneously with conventional therapeutic agents, and may be administered single or multiple times. Considering all of the above factors, it is important to administer an amount that can achieve the maximum effect with the minimum amount without side effects, and this can be easily determined by a person skilled in the art.

Specifically, the effective amount of the composition of the present disclosure may vary depending on the patient's age, sex, condition, body weight, the absorption, inactivation and excretion rate of the active ingredient in the body, disease type, and concurrently used drugs. In addition, the composition can be administered every day or every other day, 1 to 3 times a day. However, since the dosage may be increased or decreased depending on the route of administration, severity of obesity, sex, body weight, age, etc., the above dosage does not limit the scope of the present disclosure in any way.

When the composition of the present disclosure is administered, it may be formulated with a suitable amount of a pharmaceutically acceptable vehicle or carrier to provide an appropriate dosage form.

Meanwhile, the composition may further contain a carrier, an excipient, and a diluent used in the preparation of pharmaceutical compositions.

The carrier may be a commonly used one and includes, but is not limited to, saline solution, sterilized water, Ringer's solution, buffered saline, cyclodextrin, dextrose solution, maltodextrin solution, glycerol, ethanol, liposome, etc., and other common additives such as an antioxidant, a buffer, etc. may be further added as needed.

In addition, an excipient, a diluent, a dispersant, a surfactant, a binder, a lubricant, etc. can be additionally added to formulate an injectable formulation such as an aqueous solution, a suspension, an emulsion, etc., a pill, a capsule, a granule or a tablet.

The excipient or diluent may be lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate or mineral oil, although not being limited thereto.

Suitable pharmaceutically acceptable carriers and formulations can be referred to those disclosed in the Remington's literature. The pharmaceutical composition of the present disclosure is not particularly limited in formulation, but may be formulated as an injection formulation, an infusion formulation, a spray formulation, an inhalation formulation, or a formulation for external application to the skin.

Additionally, the composition can be formulated and used in the form of an oral formulation such as a powder, a granule, a tablet, a capsule, a suspension, an emulsion, a syrup, an aerosol, a formulation for external application, a suppository, and a sterile injectable solution.

Solid preparations for oral administration may include a tablet, a pill, a powder, a granule, a capsule, etc., and the solid preparation may be prepared by mixing the nanostructure according to the present disclosure with at least one excipient, e.g., starch, calcium carbonate, sucrose, lactose, gelatin, etc. Additionally, in addition to the above excipients, a lubricant such as magnesium sterate and talc may be used. Liquid preparations for oral administration may include a suspension, an oral solution, an emulsion, a syrup, etc. In addition to simple diluents such as water and liquid paraffin, various excipients such as a wetting agent, a sweetener, a fragrance, a preservative, etc. may be used.

Preparations for parenteral administration may include a sterilized aqueous solution, a non-aqueous solution, a suspension, an emulsion, a freeze-dried preparation and a suppository. For the non-aqueous solution or suspension, propylene glycol, polyethylene glycol, vegetable oil such as olive oil, or an injectable ester such as ethyl oleate may be used. As a base for the suppository, witepsol, macrogol, tween 61, cocoa butter, laurin butter and glycerogelatin may be used.

Hereinafter, the present disclosure will be described in more detail with reference to specific examples. However, these examples are only for illustrating the present disclosure in more detail, and it will be apparent to those skilled in the art that the scope of the present disclosure is not limited thereto.

### Examples and Experimental Examples

### Examples 1-1 to 1-6. Synthesis of Dendritic Polyelectrolyte Zwitterion (DPZ)

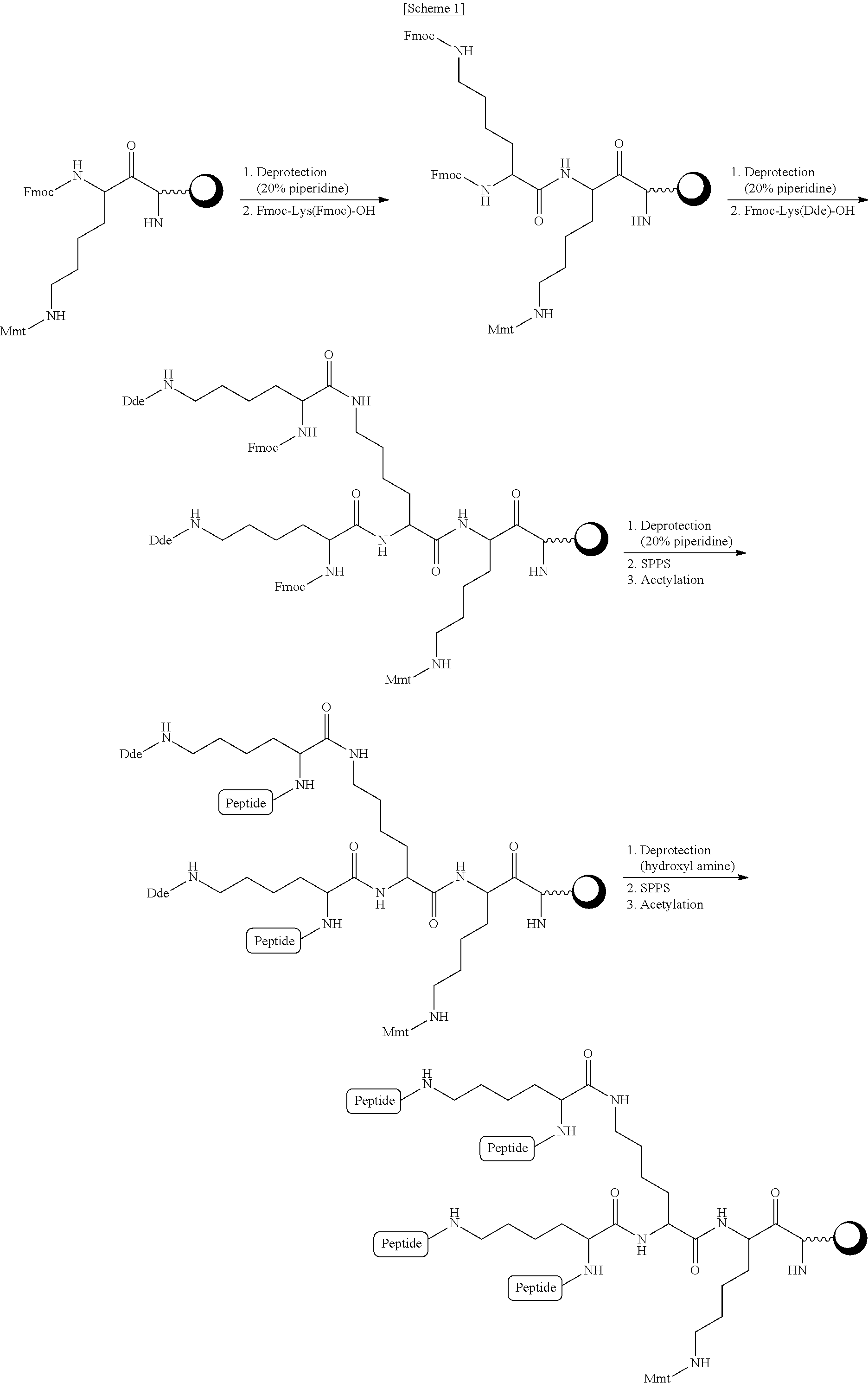

Solid-phase peptide synthesis (SPSS) allows easy synthesis of peptides, but it is limited in that hydrophobic sequences that tend to aggregate are difficult to synthesize because the aggregation reaction is not controlled due to their intrinsic hydrophobicity and hydrogen bonding. To overcome this limitation, a dendritic polyelectrolyte zwitterion (DPZ) was synthesized from lysine protected with Fmoc (9-fluorenylmethyloxycarbonyl) and Dde (4,4-dimethyl-2,6-dioxocyclohex-1-ylidene)ethyl) groups by a bifurcation synthesis method using solid-phase peptide synthesis (SPPS) according to Scheme 1.

All Fmoc-amino acids were purchased from AAPPTec (USA). A polylysine dendrimer was prepared step by step from the newly generated two amines by selective deprotection of the two Fmoc groups by piperidine and SPPS, according to Scheme 1.

Specifically, the dendritic polyelectrolyte zwitterion (DPZ) was synthesized on Rink Amide 4-methylbenzhydrylamine (MBHA) low loading (LL) resin. A 2nd generation (G2) polylysine dendrimer was synthesized by sequentially coupling Fmoc-Lys(Fmoc)-OH and Fmoc-Lys(4,4-dimethyl-2,6-dioxocyclohex-1-ylidene)ethyl, Dde)-OH on the resin. In order to attach a positively charged peptide and a negatively charged peptide to the terminal amino group of the polylysine dendrimer, the Fmoc group was removed with 20% piperidine, and two positively charged peptide strands were synthesized sequentially using SPPS. The Dde group was then removed with 2% hydrazine monohydrate solution in DMF, and the other two negatively charged peptide strands were extended by SPPS.

The positively charged peptide chain used in the DPZ synthesis was synthesized as SEQ ID NOS: 3 to 8 depending on the length of the positively charged arginine residue (Arg, R), and the negatively charged peptide chain was synthesized as SEQ ID NOS: 99 to 104 depending on the length of the negatively charged glutamic acid (Glu, E) residue.

The prepared polyelectrolyte zwitterion-based dendrimer was DPZ(+M)ₙ(−M)ₙ, wherein M is the number of amino acid residues constituting the positively charged peptide and the negatively charged peptide, and n is the number of peptide strands. M is an integer from 1 to 16, specifically from 1 to 12, more specifically from 1 to 6, and n is an integer from 1 to 10, specifically from 1 to 4, more specifically from 1 to 2. Since the positively charged peptide and the negatively charged peptide chain are linked to form the polylysine dendrimer, the dendrimer exhibits the characteristics of both a polyelectrolyte and a zwitterion and also exhibits hydrophilicity.

According to the experimental method described above, (+1)₂(−1)₂ (Example 1-1, Chemical Formula 1-1), (+2)₂(−2)₂ (Example 1-2, Chemical Formula 1-2), (+3)₂(−3)₂ (Example 1-3, Chemical Formula 1-3), (+4)₂(−4)₂ (Example 1-4, Chemical Formula 1-4), (+5)₂(−5)₂ (Example 1-5, Chemical Formula 1-5) and (+6)₂(−6)₂ (Example 1-6, Chemical Formula 1-6) were synthesized.

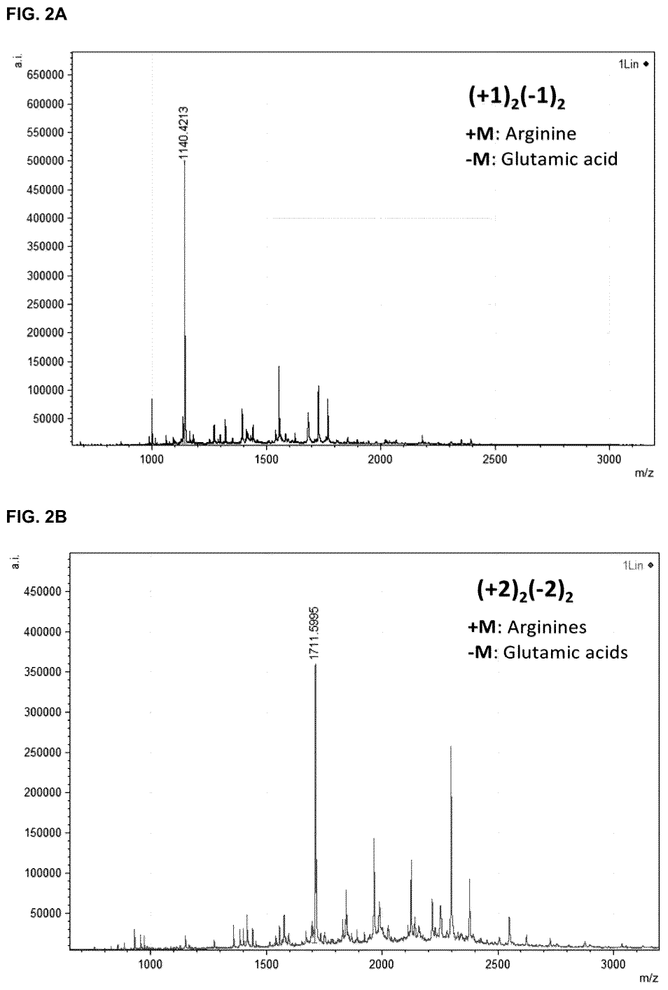

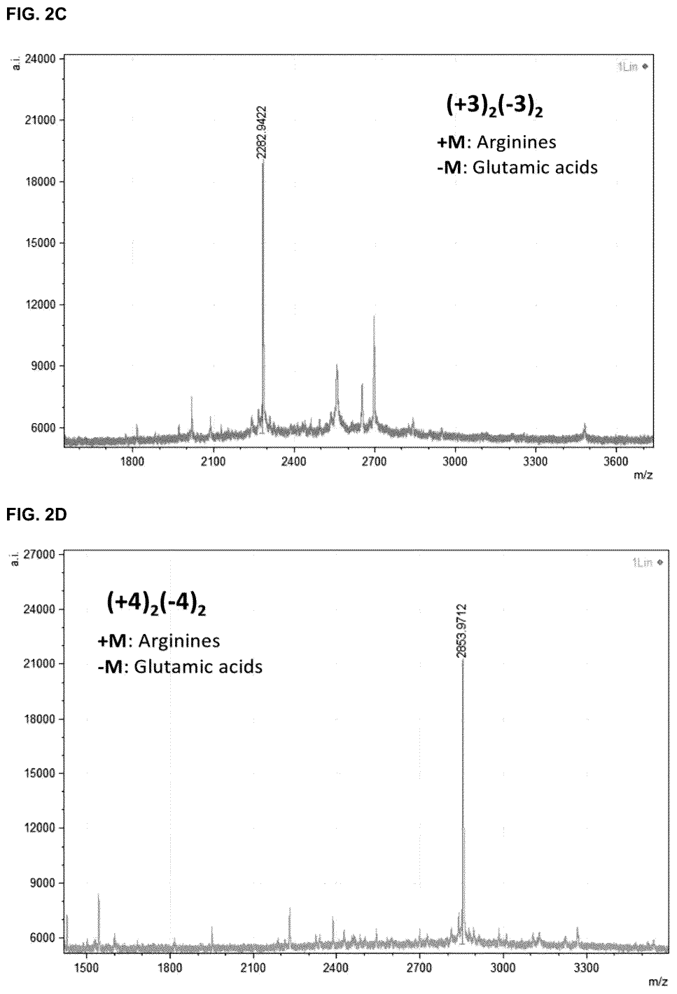

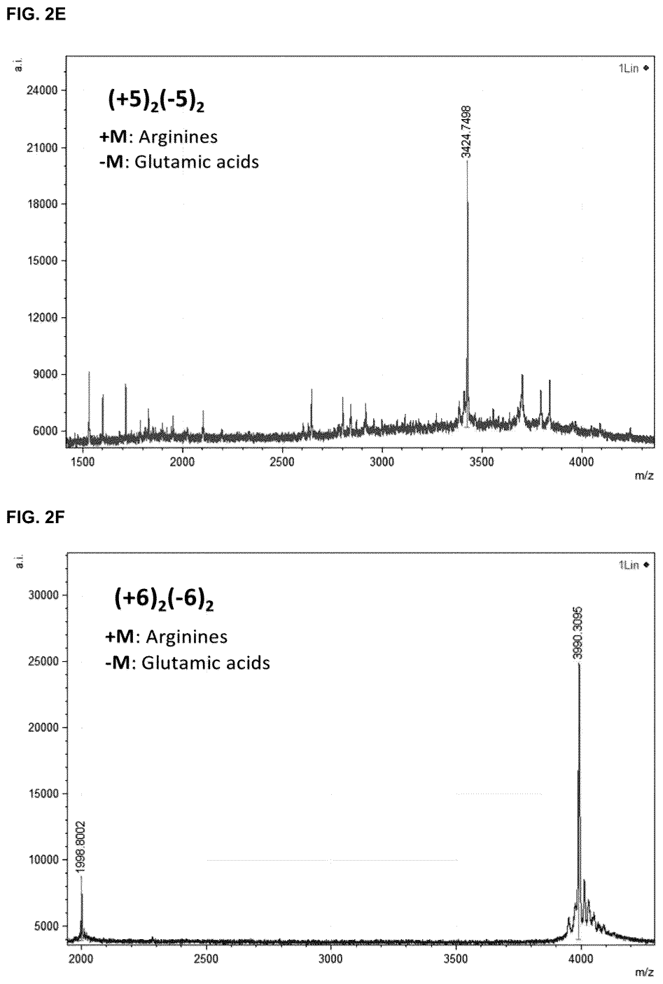

The synthesized molecule was purified by high-performance liquid chromatography (HPLC) using a C4 reversed-phase column (Waters, USA) at room temperature. Distilled water (0.1% TFA) and acetonitrile (ACN 0-100%) were used as an eluent. The molecular weight of the molecule was measured using a matrix-assisted laser desorption/ionization time-of-flight (MALDI-TOF) mass spectrometer (FIG. 2). As a result of the analytical HPLC, purity was confirmed to be >95%.

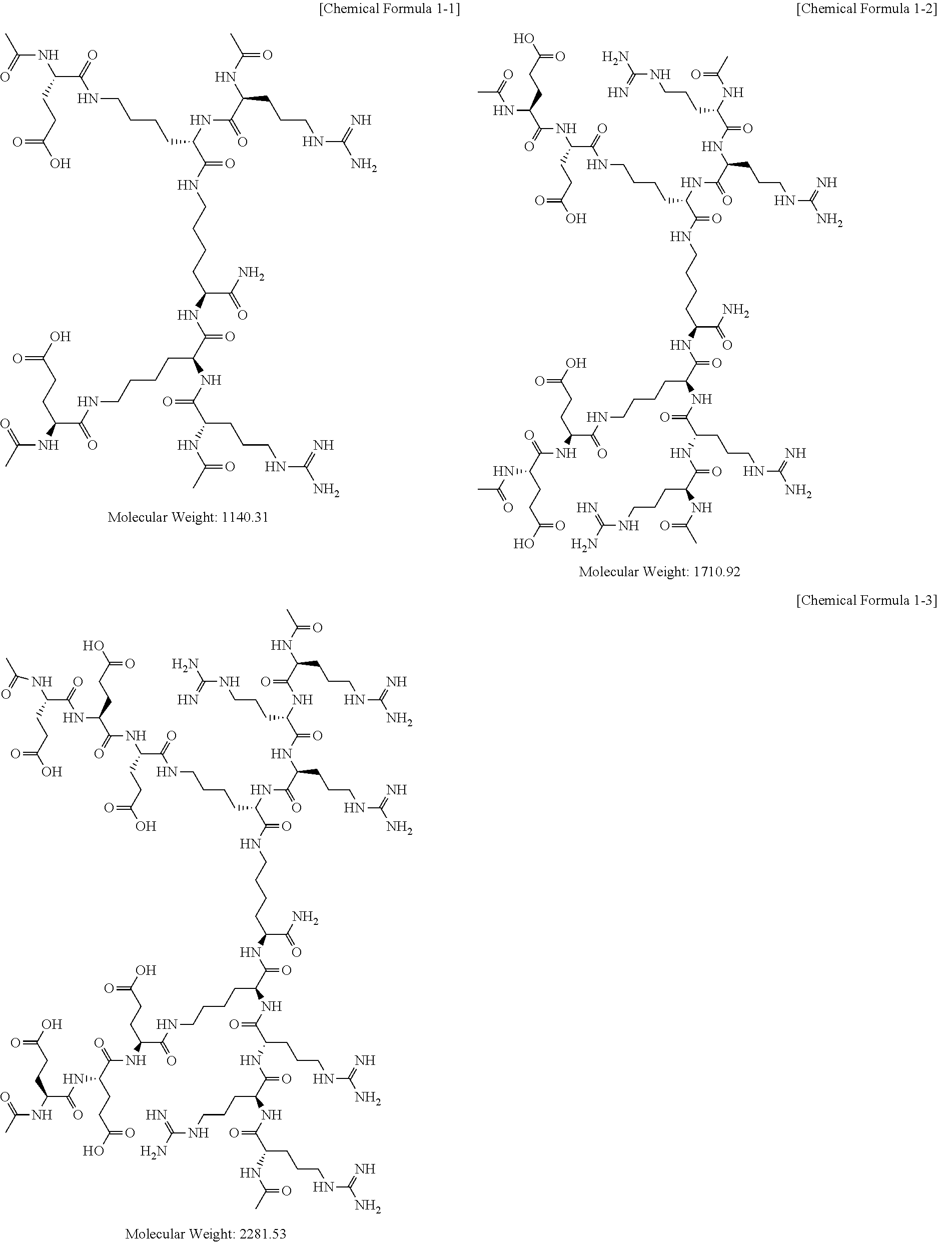

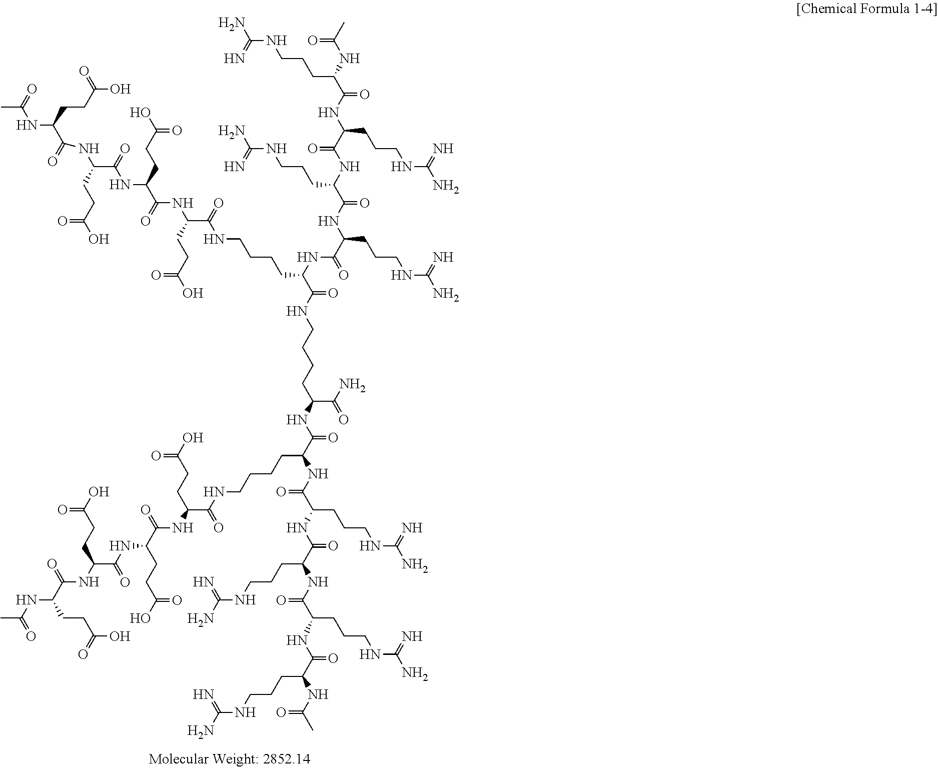 [SMILES: CC(=O)N[C@@H](CCC(=O)O)C(=O)N[C@@H](CCC(=O)O)C(=O)N[C@@H](CCC(=O)O)C(=O)N[C@@H](CCC(=O)O)C(=O)NCCCC[C@H](NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O)C(=O)NCCCC[C@H](NC(=O)[C@H](CCCCNC(=O)[C@H](CCC(=O)O)NC(=O)[C@H](CCC(=O)O)NC(=O)[C@H](CCC(=O)O)NC(=O)[C@H](CCC(=O)O)NC(C)=O)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O)C(N)=O]

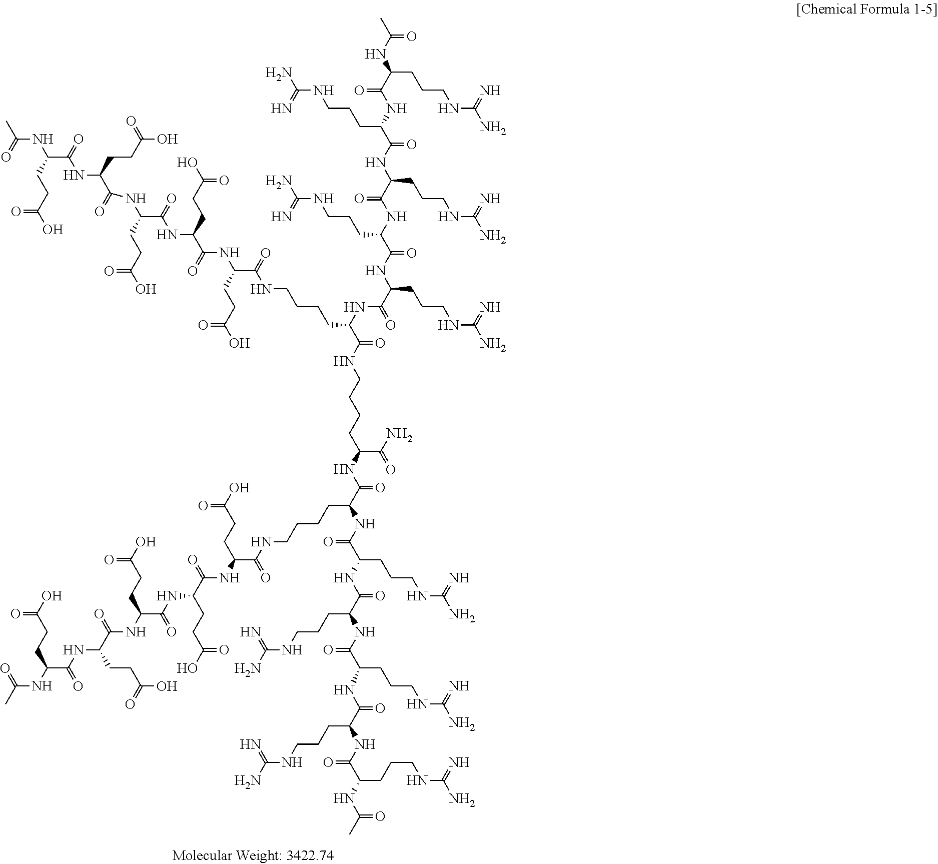

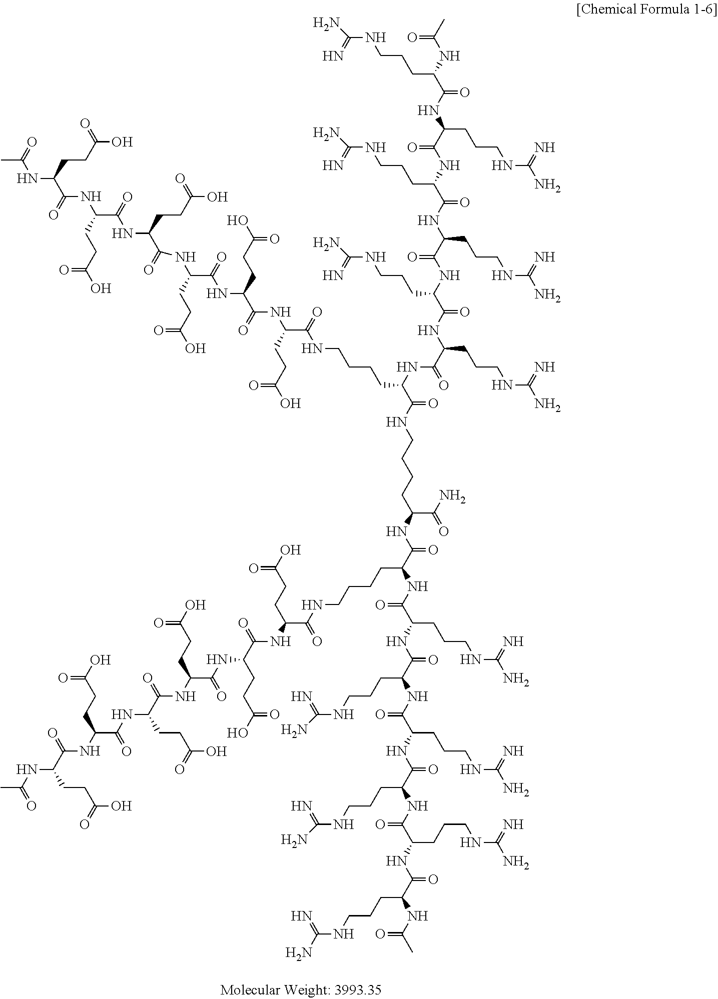

### Examples 2-1 to 2-6. Synthesis of Fluorescent Material-Introduced Dendritic Polyelectrolyte Zwitterion (DPZ)

After completing peptide chain growth as in Scheme 1, Examples 1-1 to 1-6 were synthesized in the same manner as described above, except that the Mmt group was deprotected and coupling of 5(6)-FAM (FAM=carboxyfluorescein) was performed. Through the above-described process, dendritic polyelectrolyte zwitterions (DPZ((+M)₂(−M)₂_FAM)) into which the fluorescent material FAM was introduced were synthesized (Chemical Formula Formulas 2-1 to 2-6 in order). Since bonds to various compounds can be formed through deprotection of the Mmt group, it was confirmed that the compounds applicable to the present disclosure are not particularly limited as long as they can form a bond with an amino group.

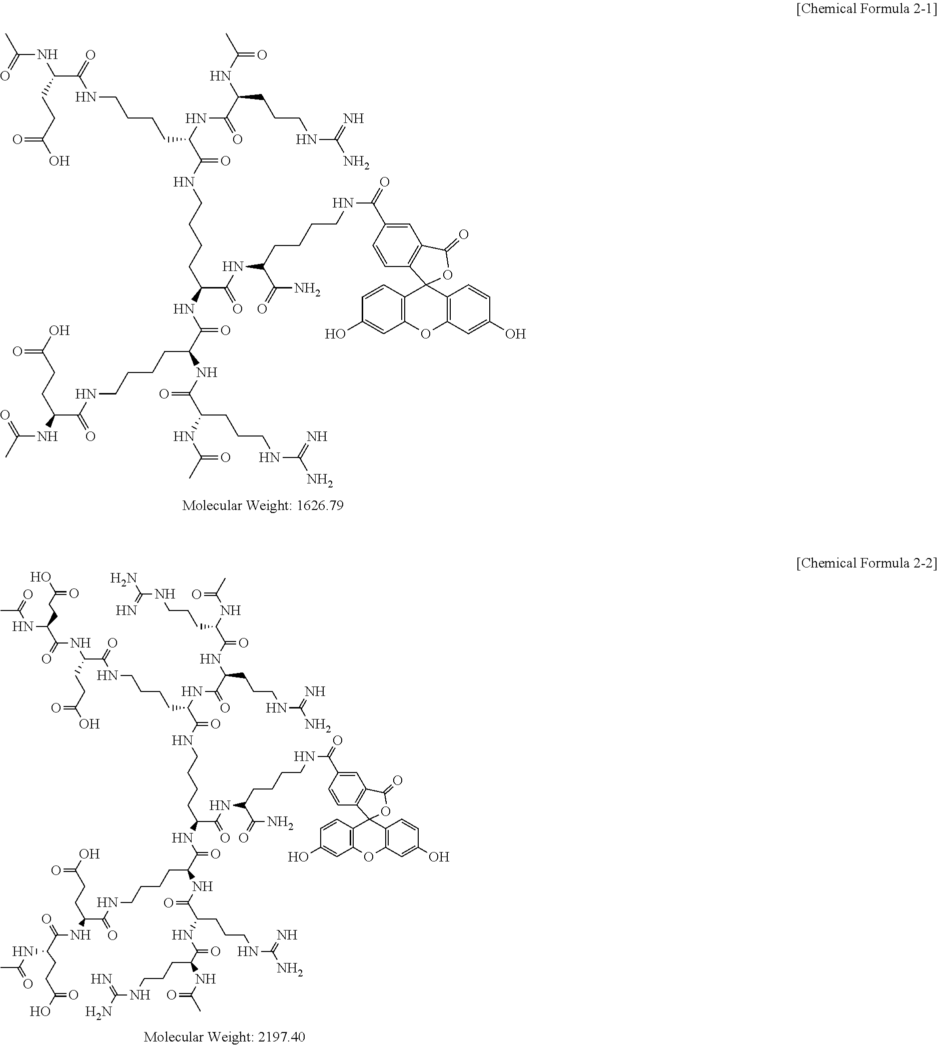

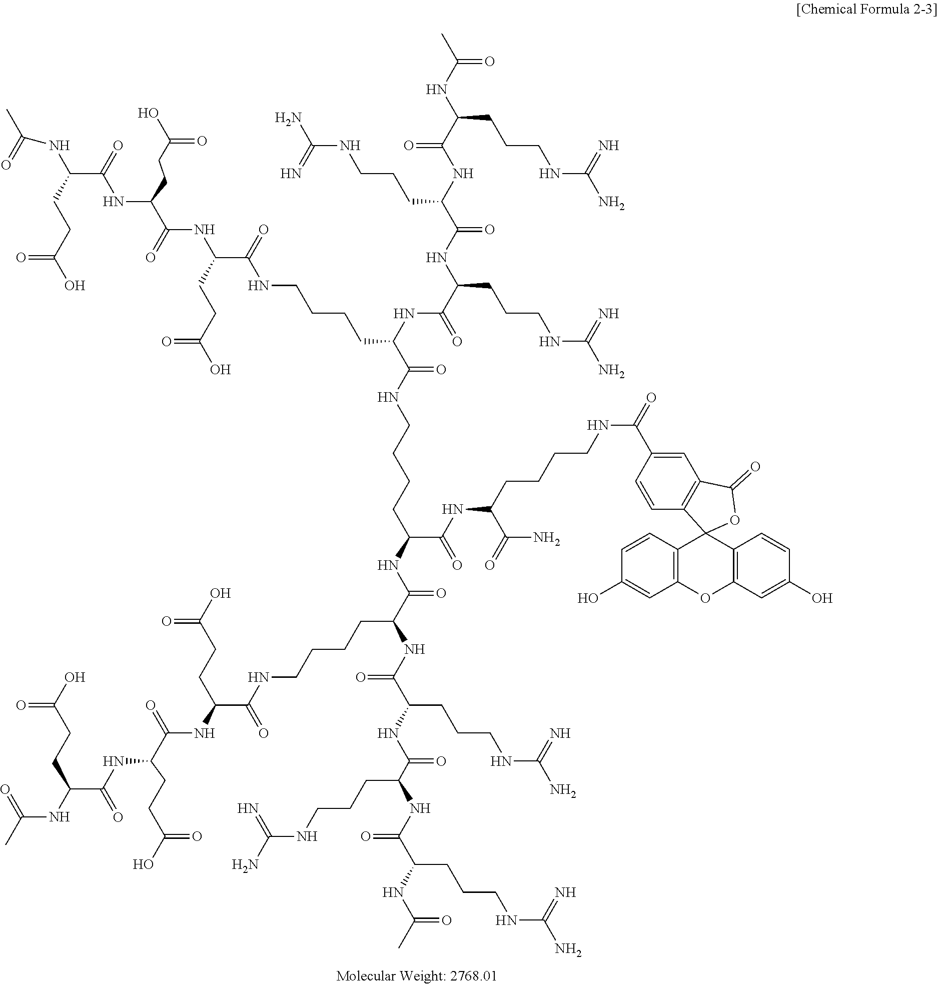

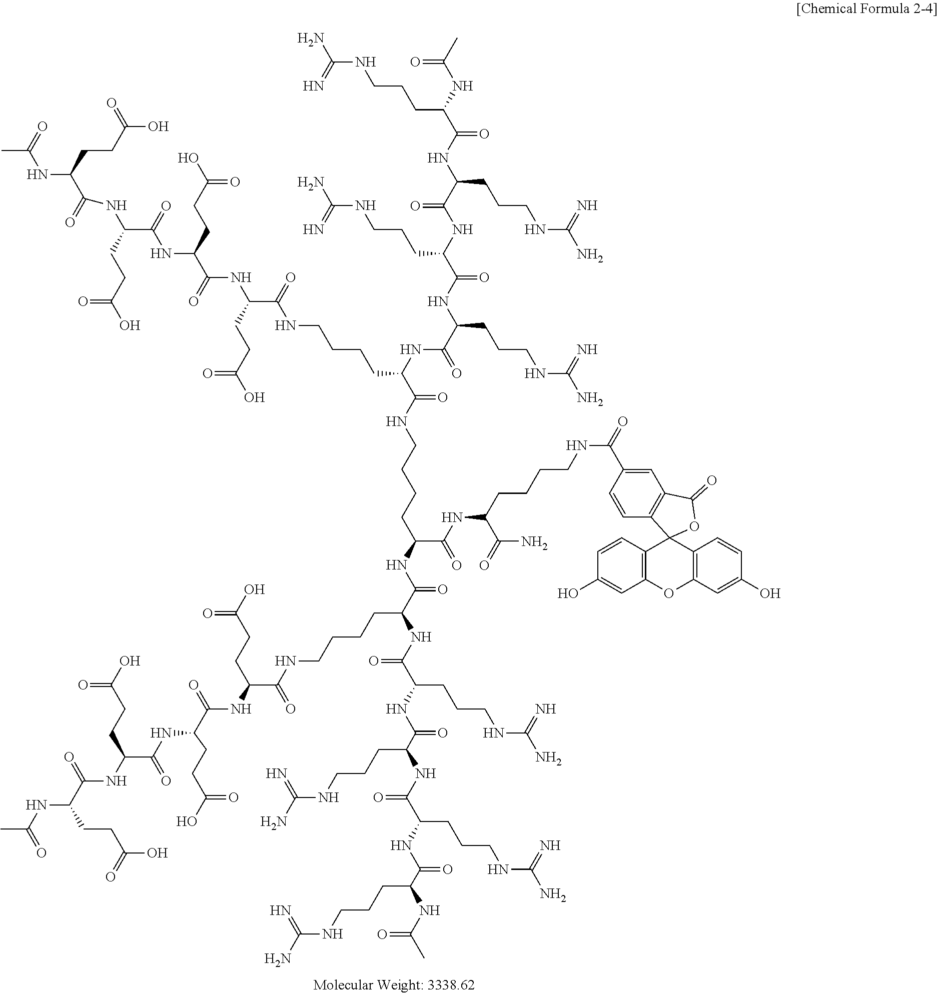

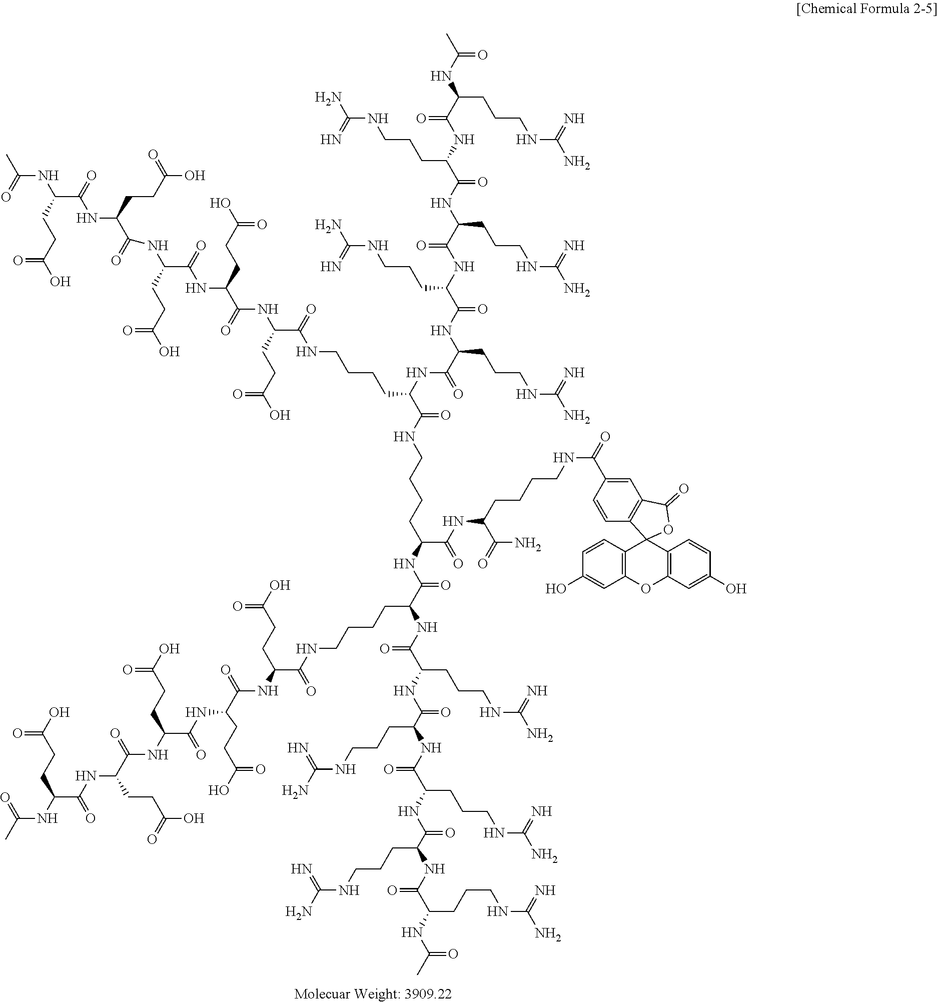

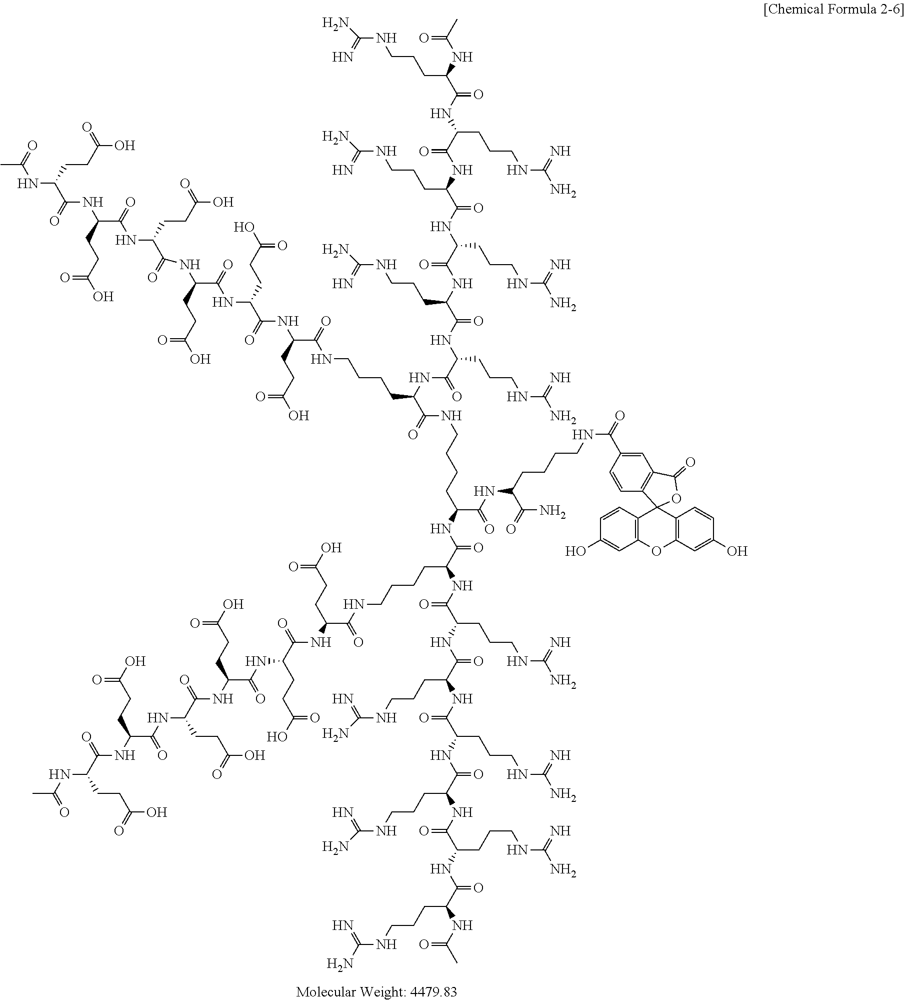

### Examples 3-1 to 3-6. G1 Janus-Type Dendritic Polyelectrolyte Zwitterion (JDPZ ((+M)₁(−M)₁-(Cₒ)ₚ)═(+M)₁(−M)₁ conjugate_1)

After synthesizing a dendritic polyelectrolyte zwitterion (DPZ) using a bifurcation synthesis method, Fmoc-Lys(Fmoc)-OH and a fatty acid alkyl chain were sequentially coupled to the amine generated from the Mmt deprotection. As a result, a Janus-type dendritic polyelectrolyte zwitterion (JDPZ ((+M)₁(−M)₁-(Cₒ)ₚ) consisting of two strands of peptide and two hydrophobic compound was synthesized (Chemical Formulas 3-1 to 3-6 in order).

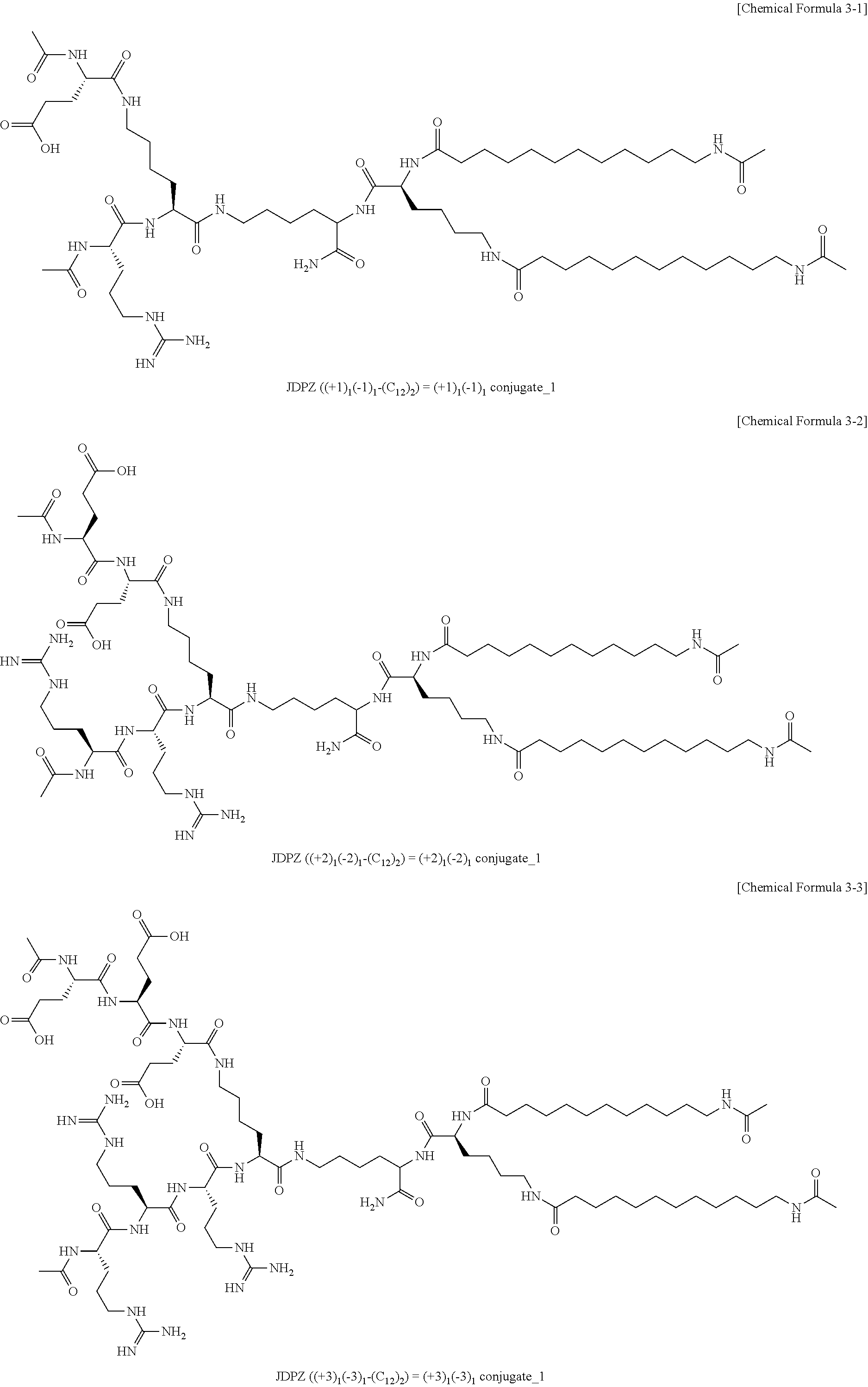

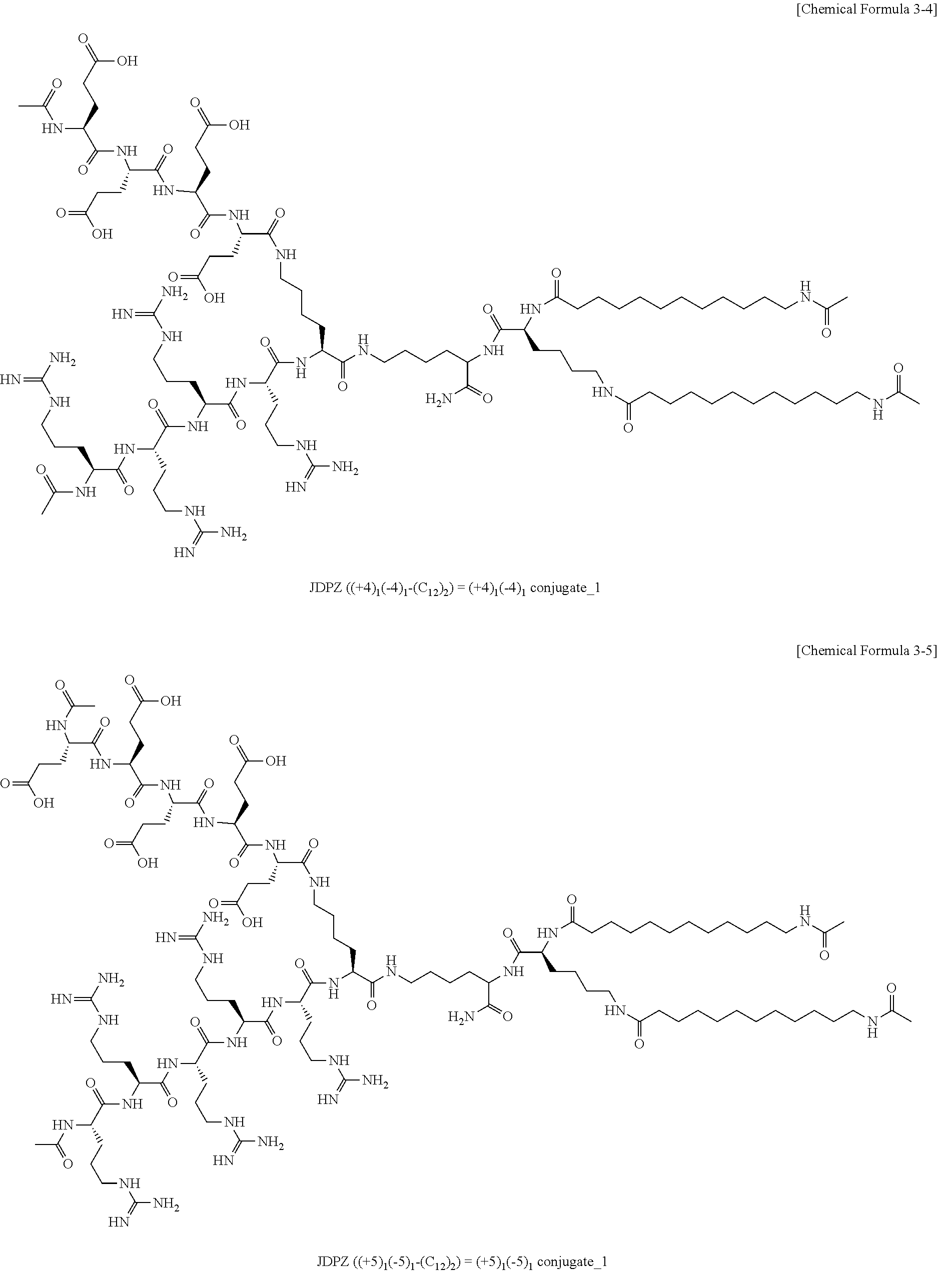

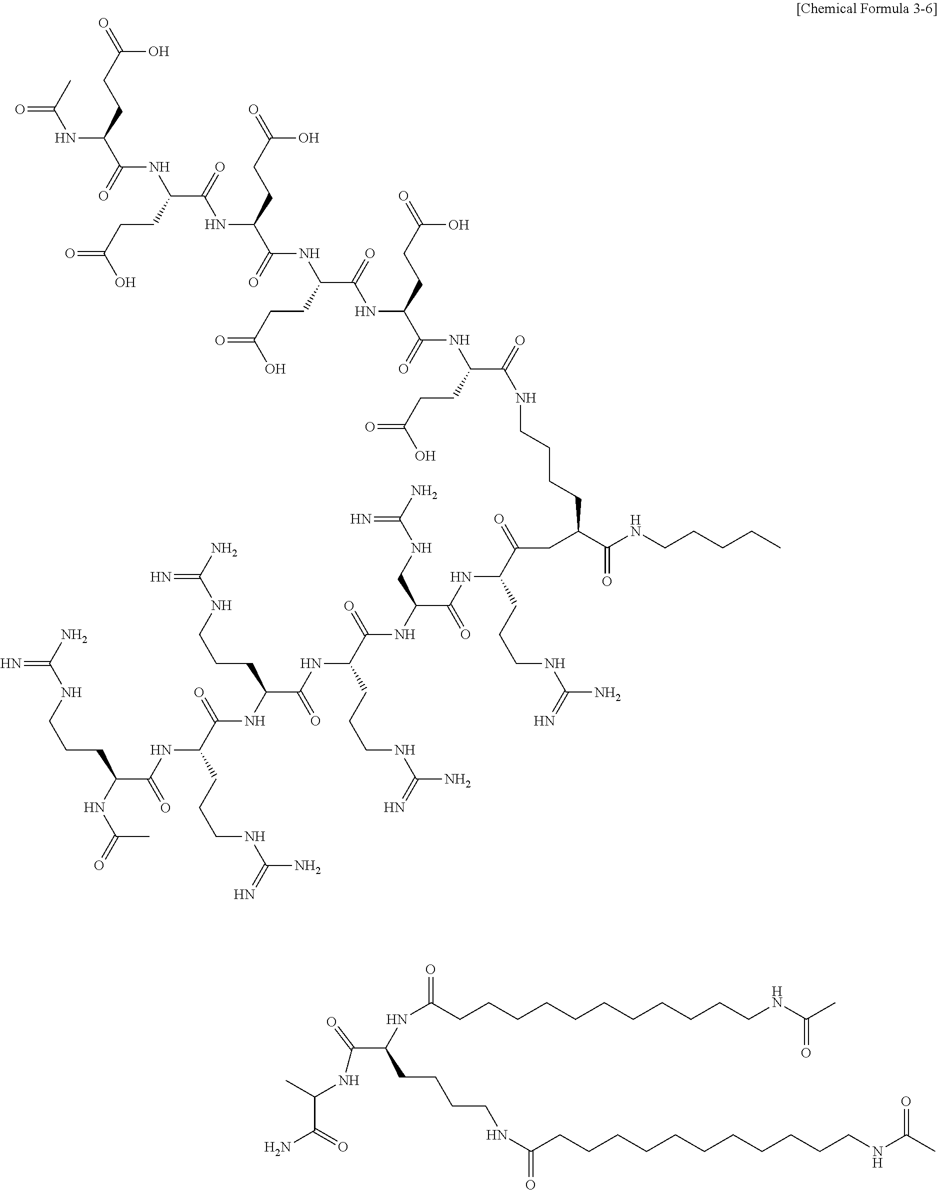 [SMILES: CCCCCNC(=O)[C@H](CCCCNC(=O)[C@H](CCC(=O)O)NC(=O)[C@H](CCC(=O)O)NC(=O)[C@H](CCC(=O)O)NC(=O)[C@H](CCC(=O)O)NC(=O)[C@H](CCC(=O)O)NC(=O)[C@H](CCC(=O)O)NC(C)=O)CC(=O)[C@H](CCCNC(=N)N)NC(=O)[C@H](CNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O ; CC(=O)NCCCCCCCCCCCC(=O)NCCCC[C@H](NC(=O)CCCCCCCCCCCNC(C)=O)C(=O)NC(C)C(N)=O]

First, or the synthesis in Rink Amide MBHA (4-methylbenzhydrylamine) LL (low loading) resin, Fmoc-Lys(Mmt)-OH and Fmoc-Lys(4,4-dimethyl-2,6-dioxocyclohex-1-ylidene)ethyl, Dde)-OH) were sequentially coupled on the resin to synthesize a 1 st generation (G1) polylysine dendrimer.

The deprotection process of the Mmt group involves treating the resin with 1% trifluoroacetic acid in dichloromethane several times (1 minute×up to 8 times) and then coupling Fmoc-Lys(Fmoc)-OH to the generated primary amine. After the deprotection of two Fmoc groups with piperidine, the primary amine was coupled using 12-aminododecanoic acid and O-(6-chlorobenzotriazol-1-yl)-N,N,N′,N′-tetramethyluronium hexafluorophosphate (HCTU), hydroxybenzotriazole (HOBt), and N,N-diisopropylethylamine (DIPEA) dissolved in NMP.

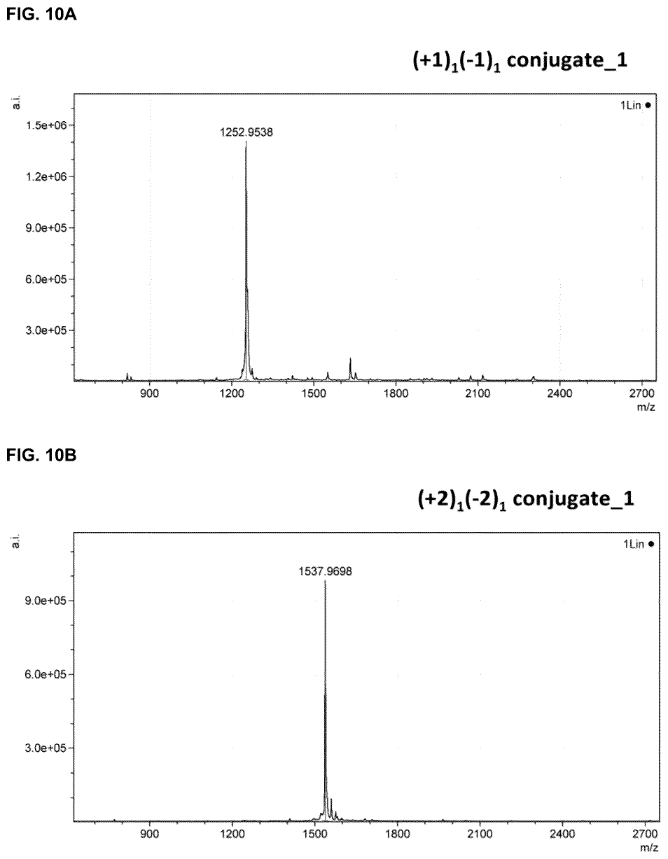

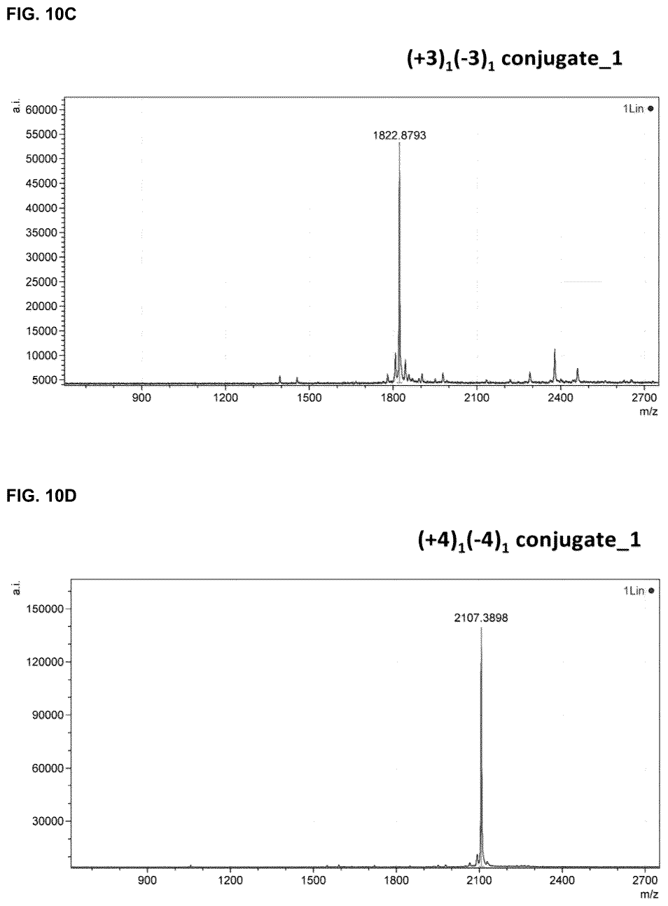

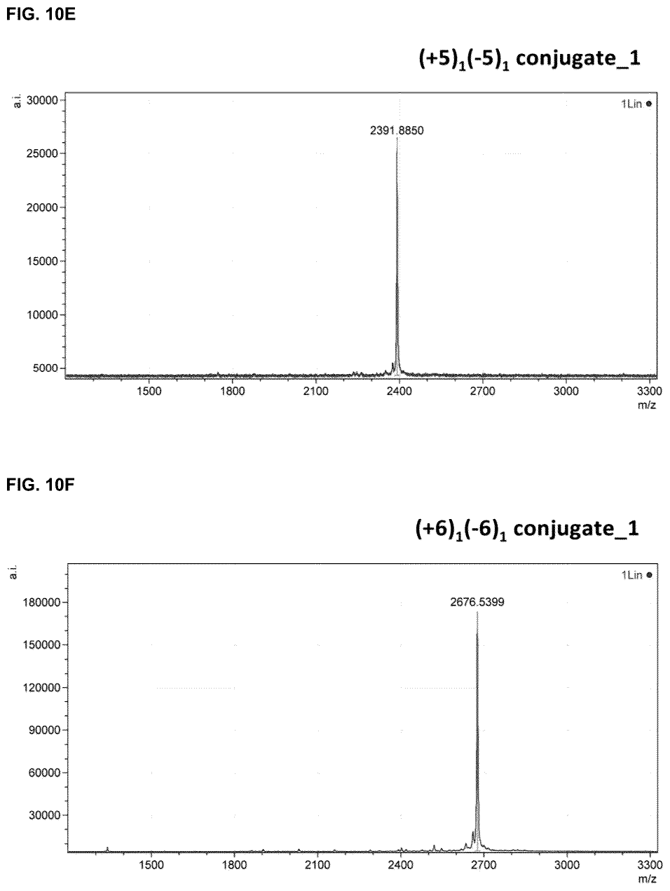

JDPZ ((+M)₁(−M)₁-(Cₒ)ₚ) (wherein M is the number of amino acid residues constituting the positively charged peptide and the negatively charged peptide, n=1 is the number of peptide strand, o is the number of carbons, and p is the number of fatty acid alkyl chains, with M being an integer from 1 to 30, o being an integer from 10 to 30, and p being an integer from 1 to 10) prepared from the above process was purified at room temperature by high-performance liquid chromatography (HPLC) using a C4 reversed-phase column (Waters, USA). Distilled water (0.1% TFA) and acetonitrile (ACN 0-100%) were used as an eluent. The molecular weight of the molecule was measured using a matrix-assisted laser desorption/ionization time-of-flight (MALDI-TOF) mass spectrometer and the result is shown in FIG. 10. As a result of the analytical HPLC, purity was confirmed to be >95%.

### Examples 4-1 to 4-12. Fluorescent Material-Introduced G1 Janus-Type Dendritic polyelectrolyte zwitterion (JDPZ ((+M)₁(−M)₁-(Cₒ)ₚ_FAM)=(+M)₁(−M)₁ conjugate_FAM)

A dendritic polyelectrolyte zwitterion (DPZ) was synthesized using a bifurcation synthesis method, and then Dde-Lys(Fmoc)-OH was coupled to the amine generated from Mmt deprotection. First, the Fmoc group was deprotected selectively and then the fatty acid alkyl chain was coupled. Then, after deprotecting the Dde group, a Janus-type dendritic polyelectrolyte zwitterion consisting of two strands of a peptide, two hydrophobic compounds, and FAM bound to the end of the hydrophobic compound (JDPZ ((+M)₁(−M)₁-(Cₒ)ₚ_FAM)) was synthesized by sequentially coupling the fatty acid alkyl chain and the fluorescent material 5(6)-FAM (Chemical Formulas 4-1 to 4-12 in order).

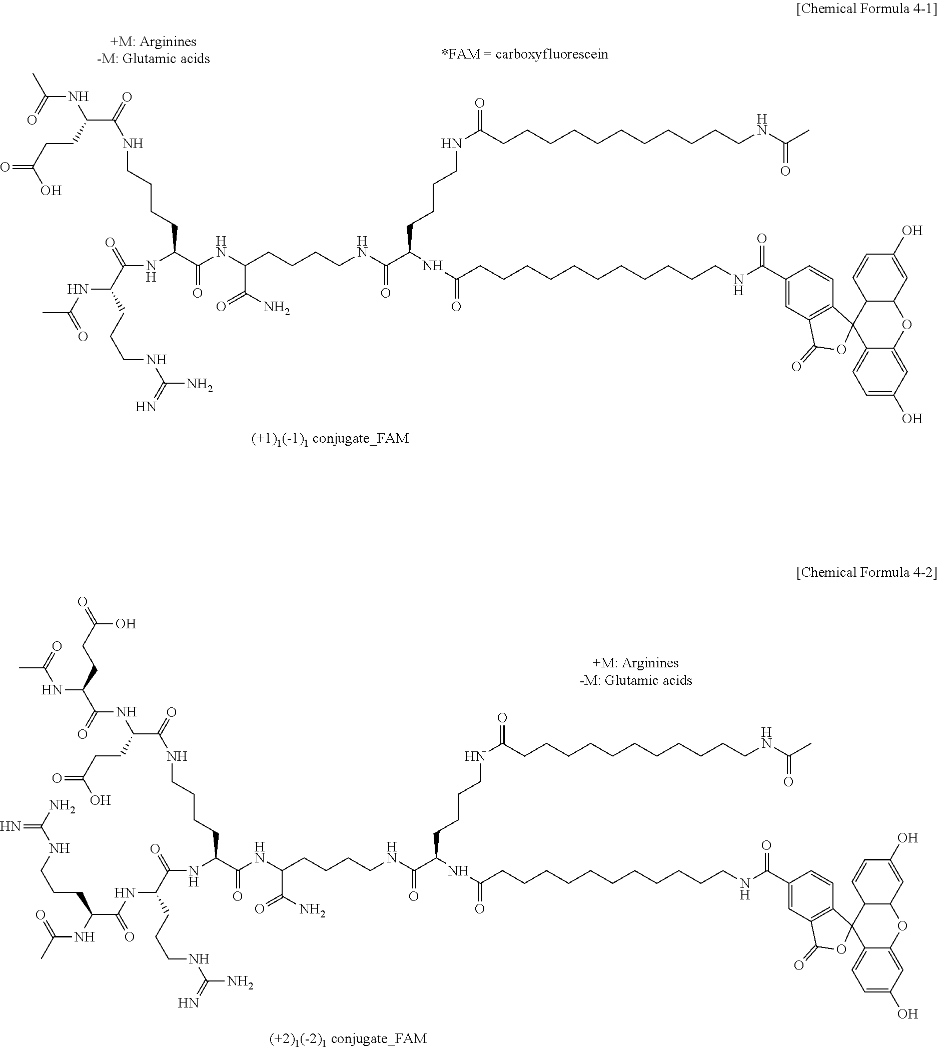 [SMILES: CC(=O)NCCCCCCCCCCCC(=O)NCCCC[C@@H](NC(=O)CCCCCCCCCCCNC(=O)c1ccc2c(c1)C(=O)OC21c2ccc(O)cc2OC2C=C(O)C=CC21)C(=O)NCCCCC(NC(=O)[C@H](CCCCNC(=O)[C@H](CCC(=O)O)NC(C)=O)NC(=O)[C@H](CCCNC(=N)N)NC(C)=O)C(N)=O ; CC(=O)NCCCCCCCCCCCC(=O)NCCCC[C@@H](NC(=O)CCCCCCCCCCCNC(=O)c1ccc2c(c1)C(=O)OC21c2ccc(O)cc2OC2C=C(O)C=CC21)C(=O)NCCCCC(NC(=O)[C@H](CCCCNC(=O)[C@H](CCC(=O)O)NC(=O)[C@H](CCC(=O)O)NC(C)=O)NC(=O)[C@H](CCCNC(=N)N)NC(=O)[C@H](CCCNC(=N)N)NC(C)=O)C(N)=O]

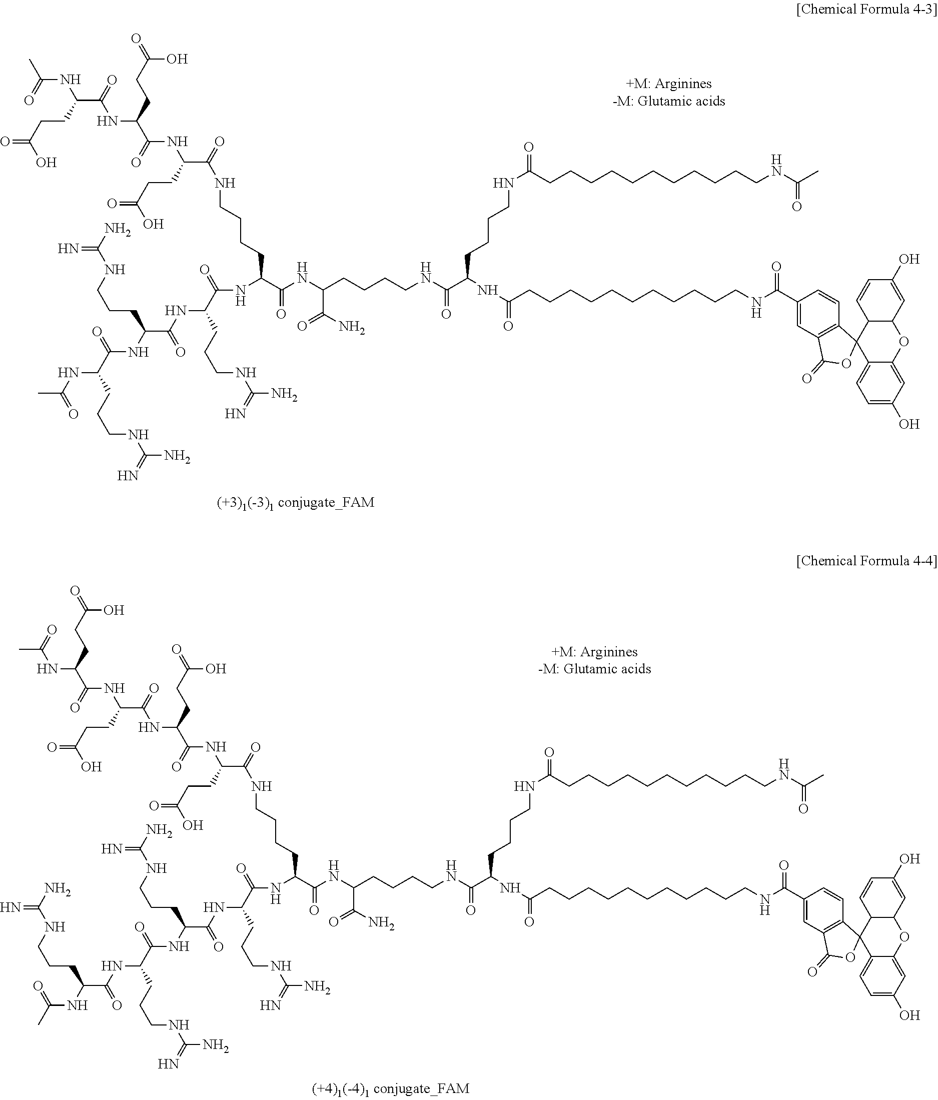

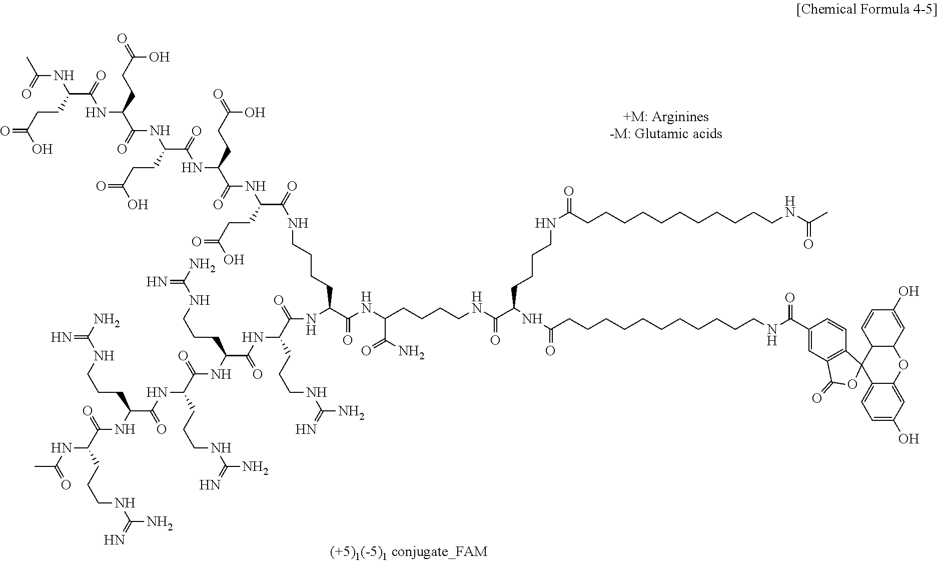 [SMILES: CC(=O)NCCCCCCCCCCCC(=O)NCCCC[C@@H](NC(=O)CCCCCCCCCCCNC(=O)c1ccc2c(c1)C(=O)OC21c2ccc(O)cc2OC2C=C(O)C=CC21)C(=O)NCCCCC(NC(=O)[C@H](CCCCNC(=O)[C@H](CCC(=O)O)NC(=O)[C@H](CCC(=O)O)NC(=O)[C@H](CCC(=O)O)NC(=O)[C@H](CCC(=O)O)NC(=O)[C@H](CCC(=O)O)NC(C)=O)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O)C(N)=O]

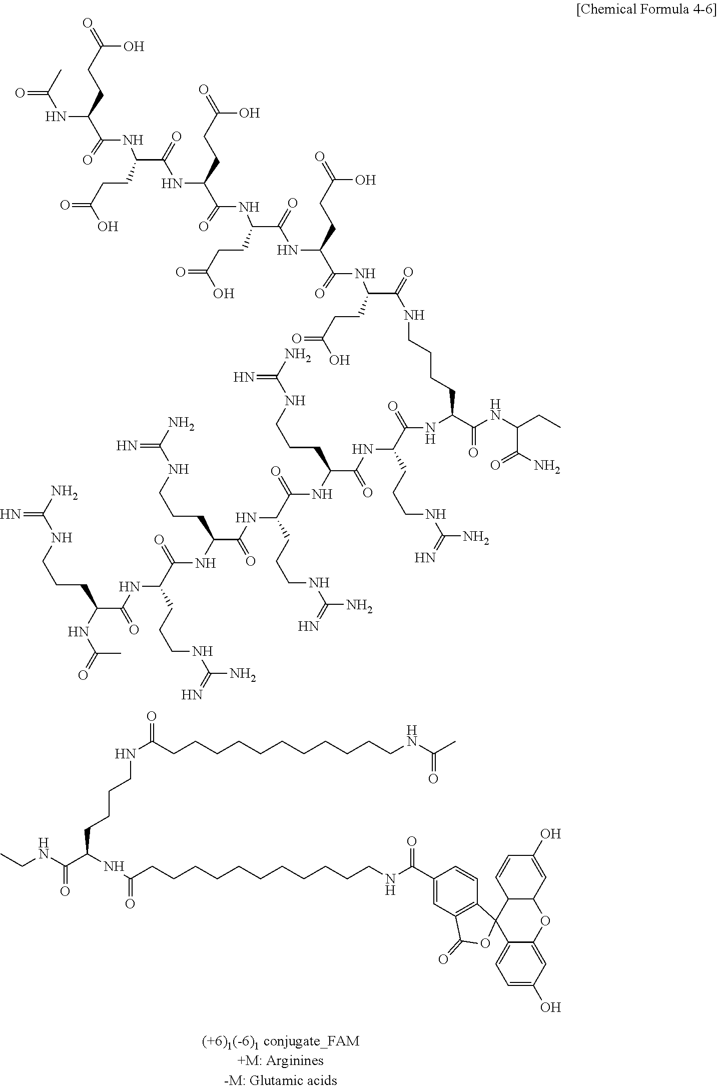

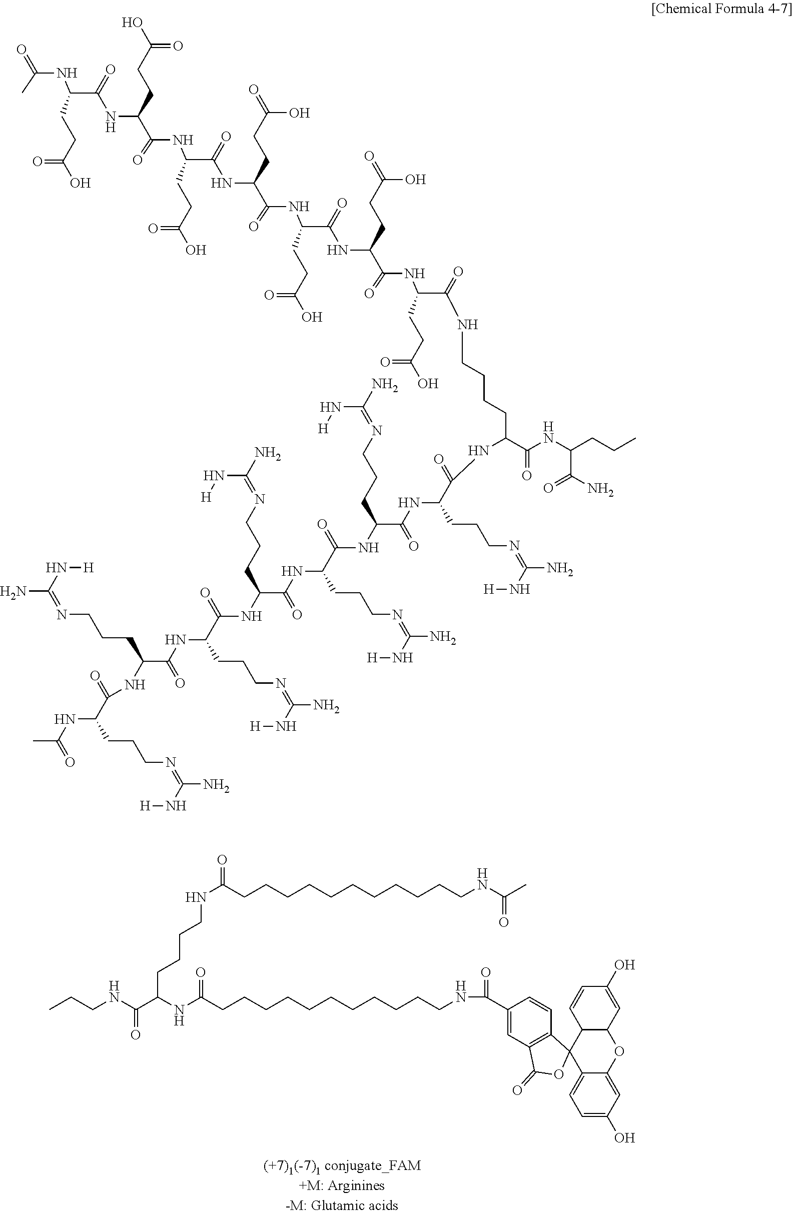

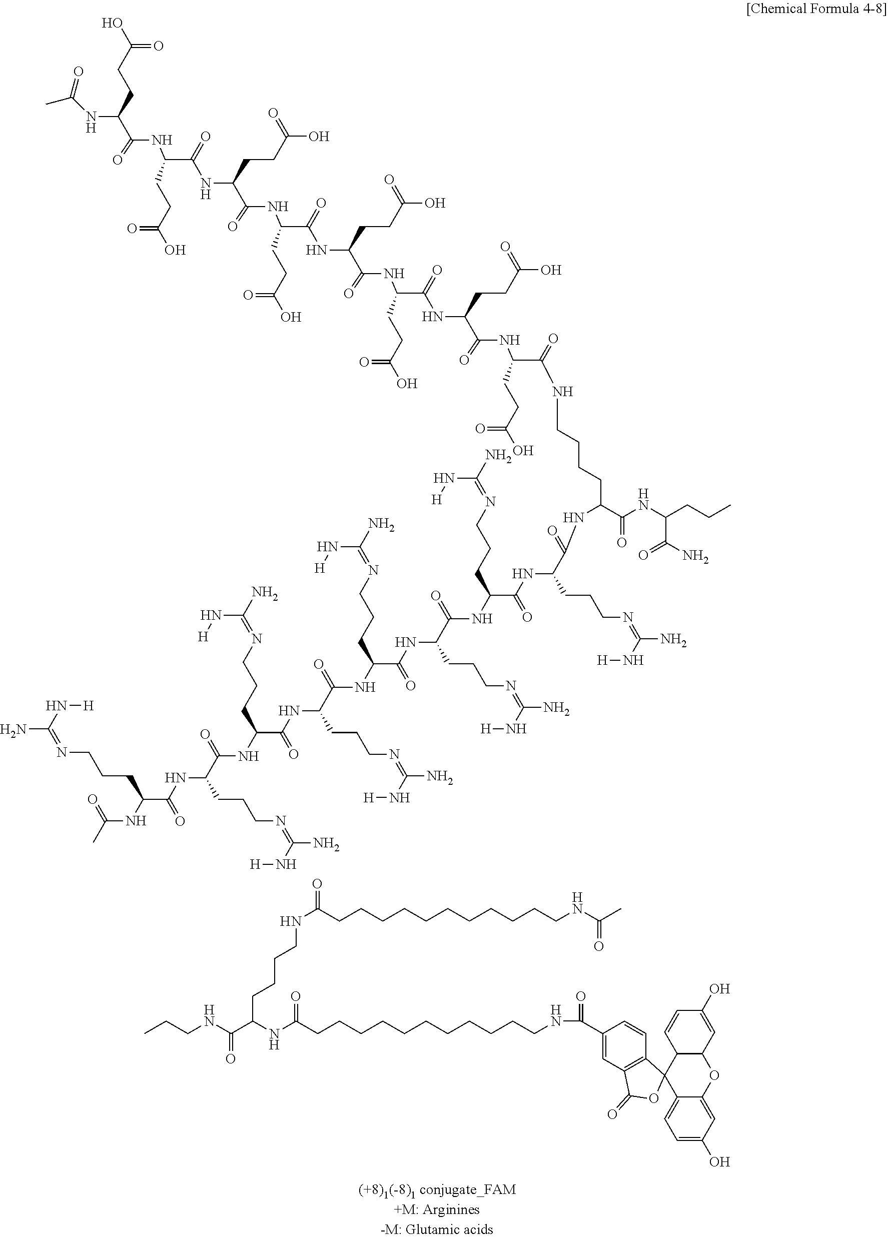

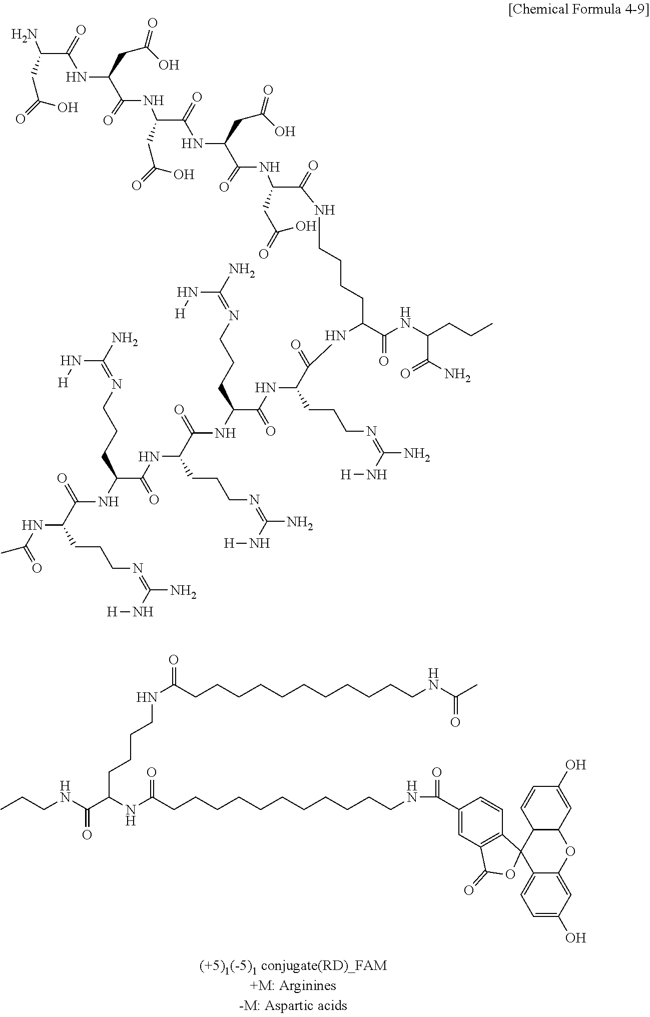

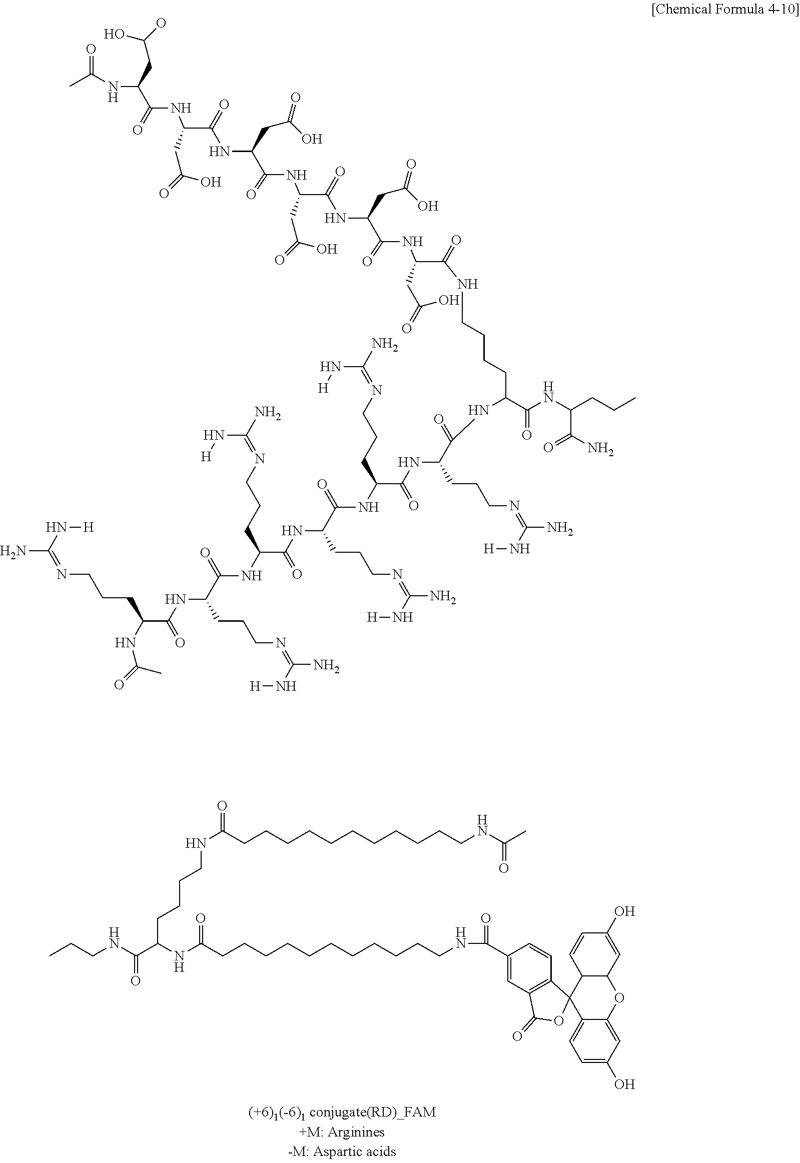

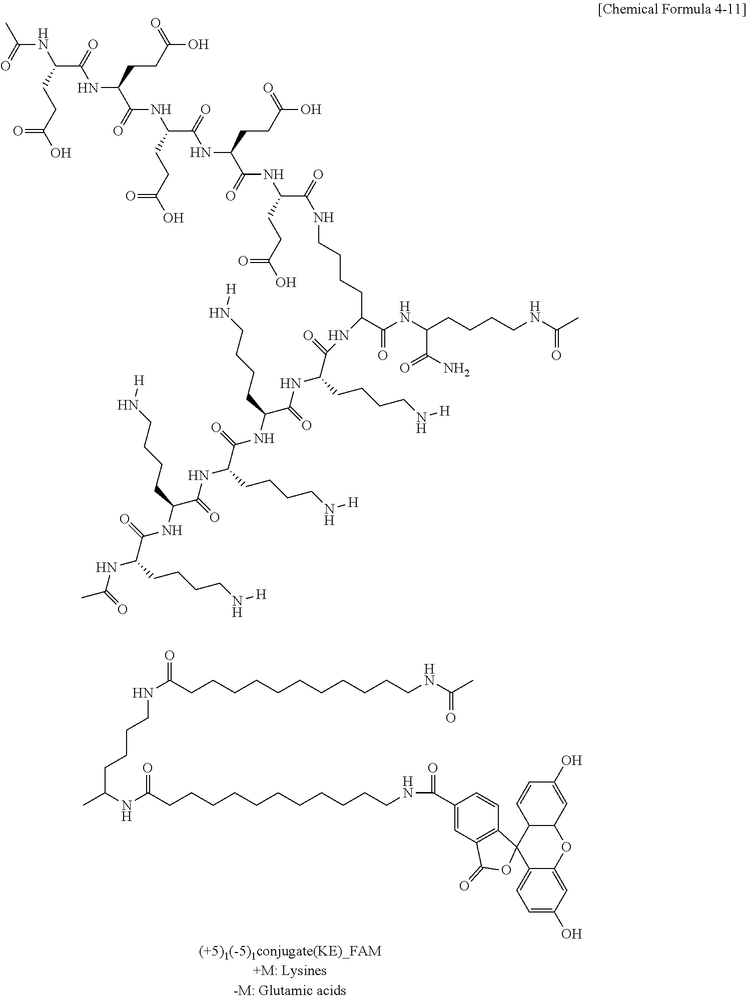

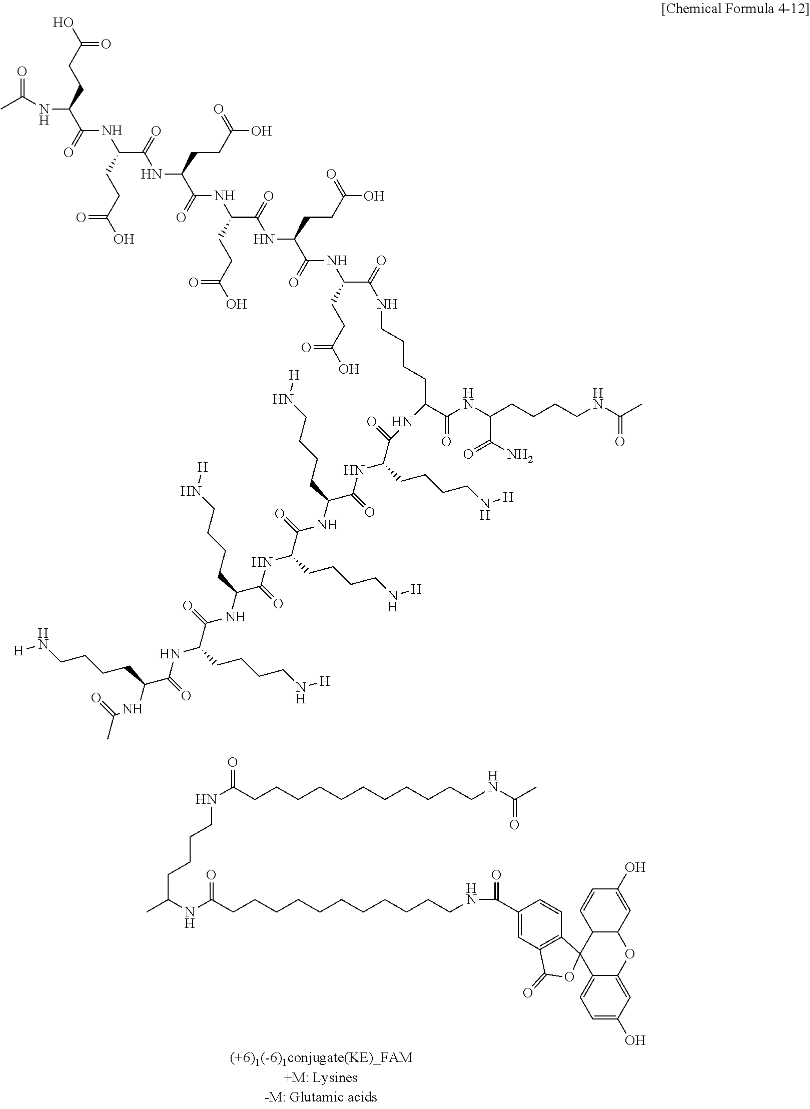

### Examples 5-1 to 5-4. G2 Janus-type dendritic polyelectrolyte zwitterion (JDPZ ((+M)₂(−M)₂-(Cₒ)ₚ)═(+M)₂(−M)₂ conjugate_1)

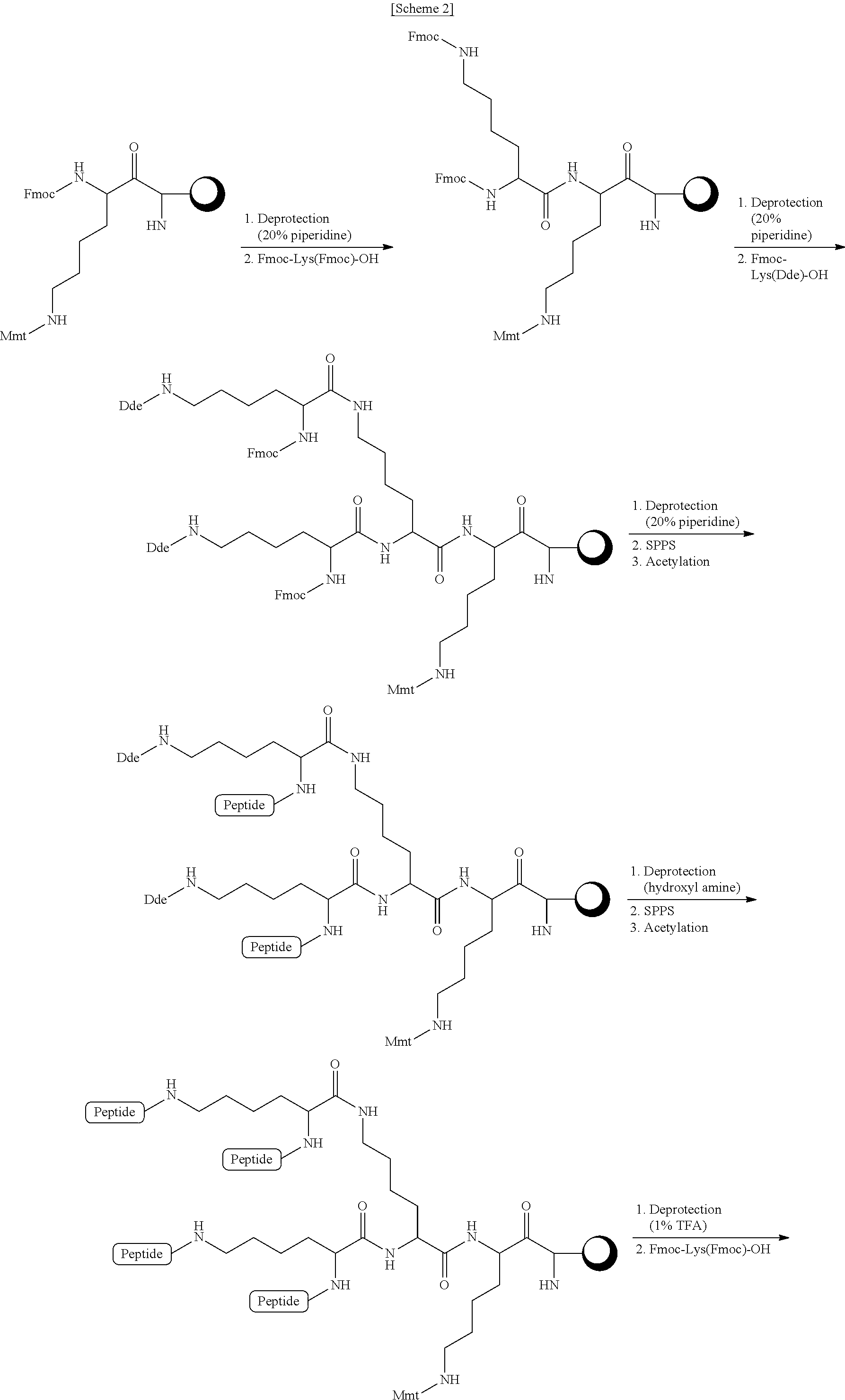

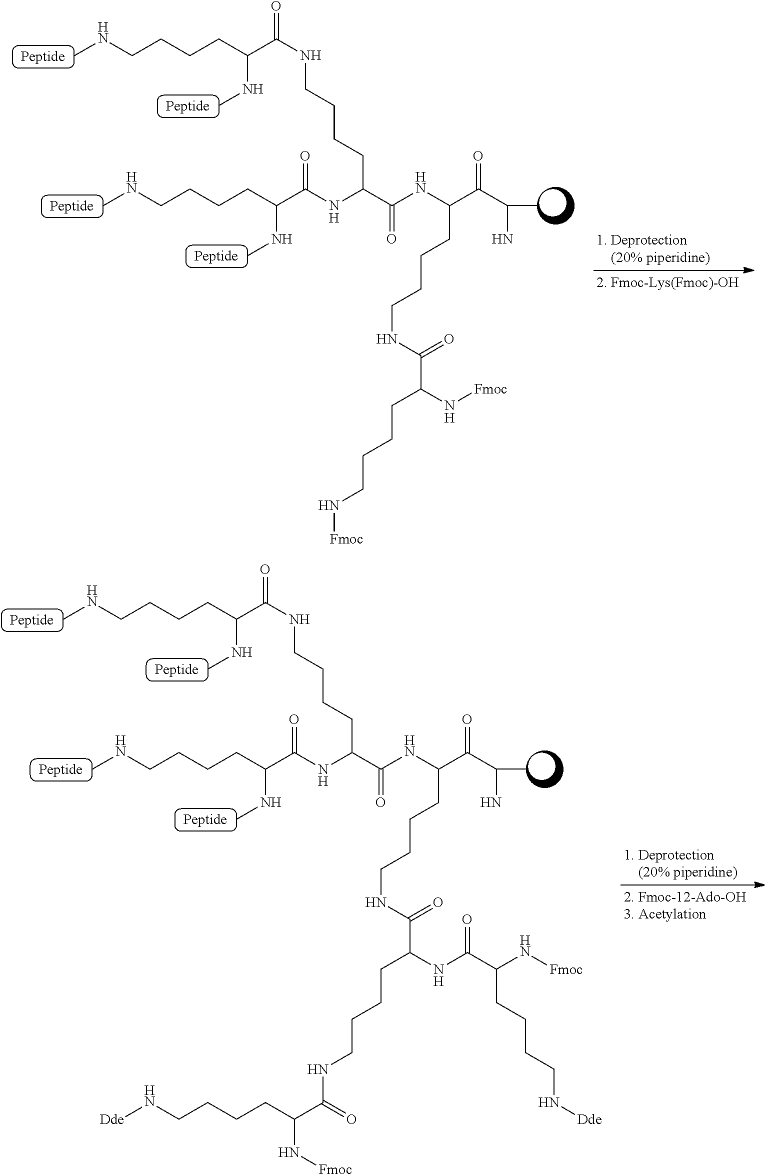

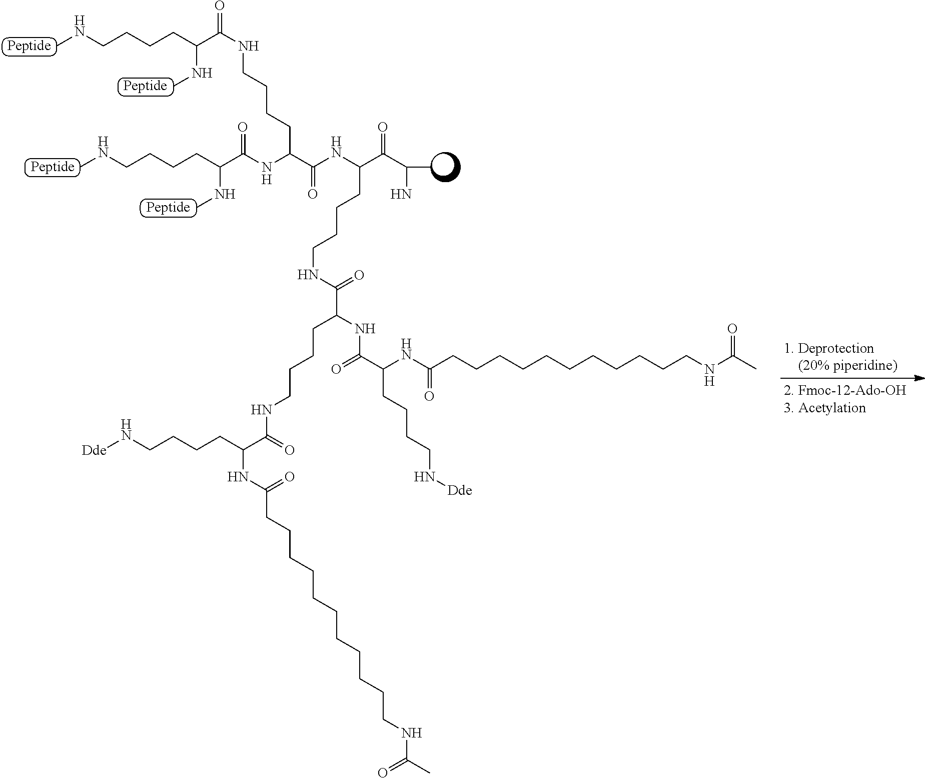

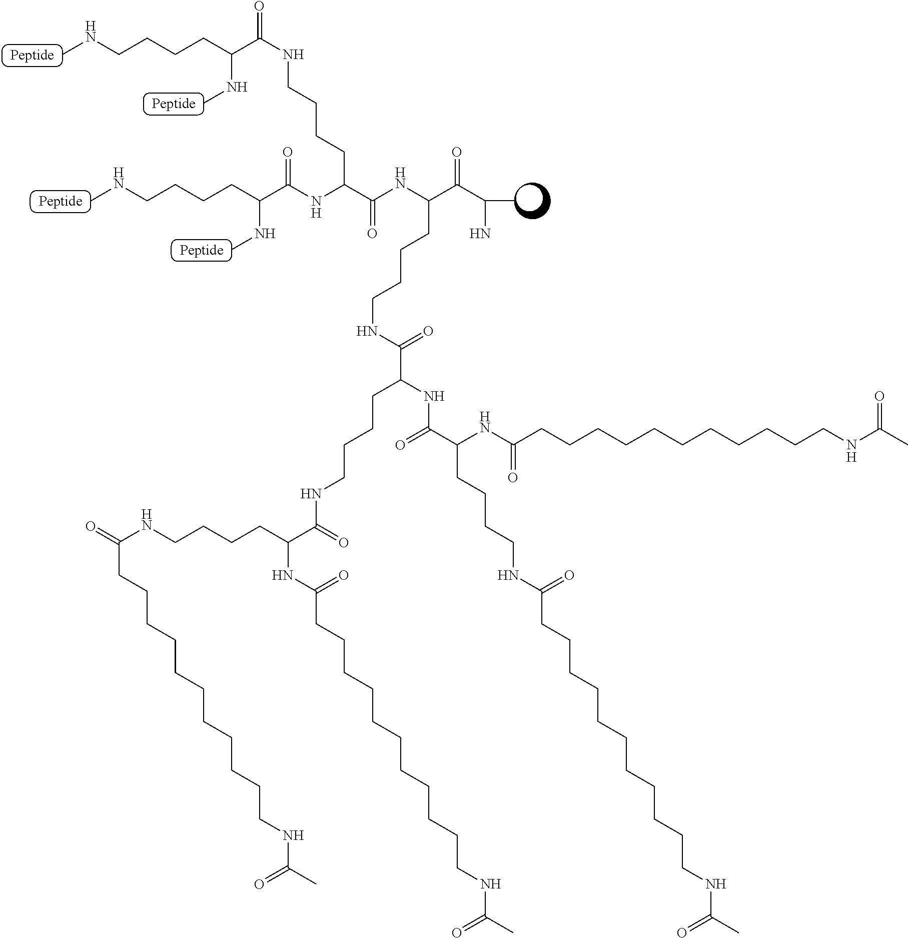 [SMILES: CNCCCCC(NC)C(=O)NCCCCC(NC(=O)C(CCCCNC)NC)C(=O)NC(CCCCNC(=O)C(CCCCNC(=O)C(CCCCNC(=O)CCCCCCCCCCCNC(C)=O)NC(=O)CCCCCCCCCCCNC(C)=O)NC(=O)C(CCCCNC(=O)CCCCCCCCCCCNC(C)=O)NC(=O)CCCCCCCCCCCNC(C)=O)C(=O)C(C)N]

A dendritic polyelectrolyte zwitterion (dendritic polyelectrolyte zwitterion, DPZ) was synthesized using the bifurcation synthesis method. The DPZ was synthesized on Rink Amide MBHA (4-Methylbenzhydrylamine) LL (low loading) resin, and a 2nd generation (G2) polylysine dendrimer was synthesized by sequentially coupling Fmoc-Lys(Mmt)-OH, Fmoc-Lys(Fmoc)-OH and Fmoc-Lys(4,4-dimethyl-2,6-dioxocyclohex-1-ylidene)ethyl, Dde)-OH. In order to attach a positively charged peptide and a negatively charged peptide to the terminal amino group of the polylysine dendrimer, the Fmoc group was removed with 20% piperidine, and two positively charged peptide strands were synthesized sequentially using SPPS. The Dde group was then removed with 2% hydrazine monohydrate solution in DMF, and the other two negatively charged peptide strands were extended by SPPS.

Afterwards, Fmoc-Lys(Fmoc)-OH was coupled to the amine generated from Mmt deprotection. After the deprotection of two Fmoc groups with piperidine, Fmoc-Lys(Dde)-OH was coupled to each amine group. After removing the Fmoc group with 20% piperidine, two fatty acid alkyl chains were coupled, and then after removing the Dde group, two fatty acid alkyl chains were coupled to synthesize a Janus-type dendritic polyelectrolyte zwitterion (JDPZ ((+M)₂(−M)₂-(Cₒ)ₚ)) consisting of four strands of peptide and four hydrophobic compounds (Chemical Formulas 5-1 to 5−4 in order).

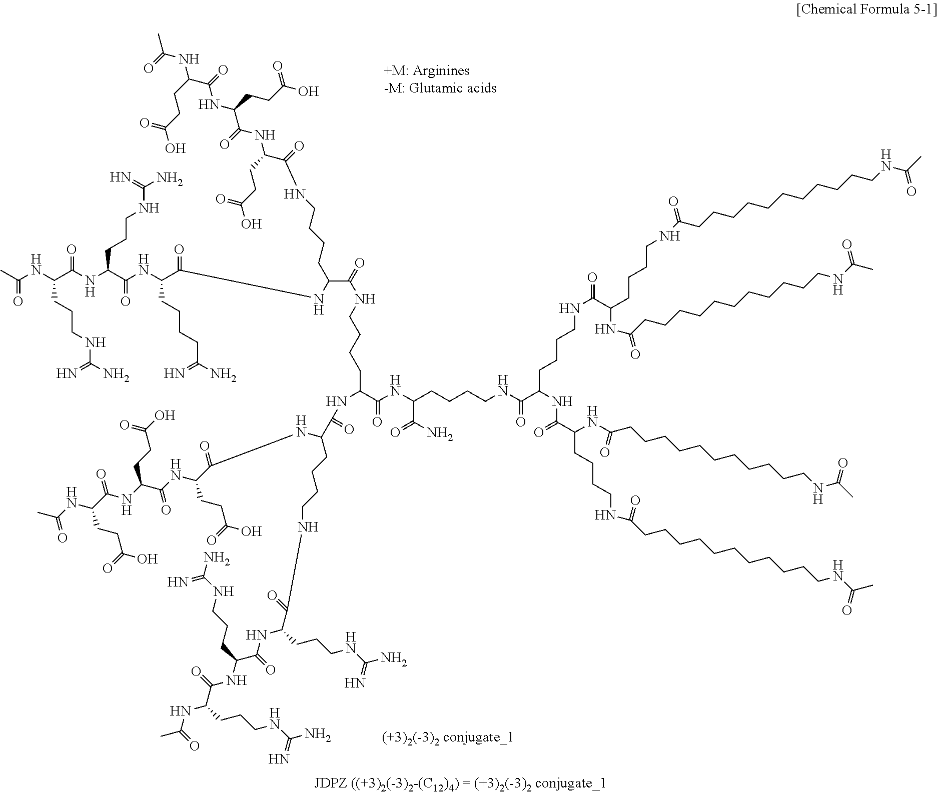 [SMILES: CC(=O)NCCCCCCCCCCCC(=O)NCCCCC(NC(=O)CCCCCCCCCCCNC(C)=O)C(=O)NCCCCC(NC(=O)C(CCCCNC(=O)CCCCCCCCCCCNC(C)=O)NC(=O)CCCCCCCCCCCNC(C)=O)C(=O)NCCCCC(NC(=O)C(CCCCNC(=O)C(CCCCNC(=O)[C@H](CCC(=O)O)NC(=O)[C@H](CCC(=O)O)NC(=O)C(CCC(=O)O)NC(C)=O)NC(=O)[C@H](CCCCC(=N)N)NC(=O)[C@H](CCCNC(=N)N)NC(=O)[C@H](CCCNC(=N)N)NC(C)=O)NC(=O)C(CCCCNC(=O)[C@H](CCCNC(=N)N)NC(=O)[C@H](CCCNC(=N)N)NC(=O)[C@H](CCCNC(=N)N)NC(C)=O)NC(=O)[C@H](CCC(=O)O)NC(=O)[C@H](CCC(=O)O)NC(=O)[C@H](CCC(=O)O)NC(C)=O)C(N)=O]

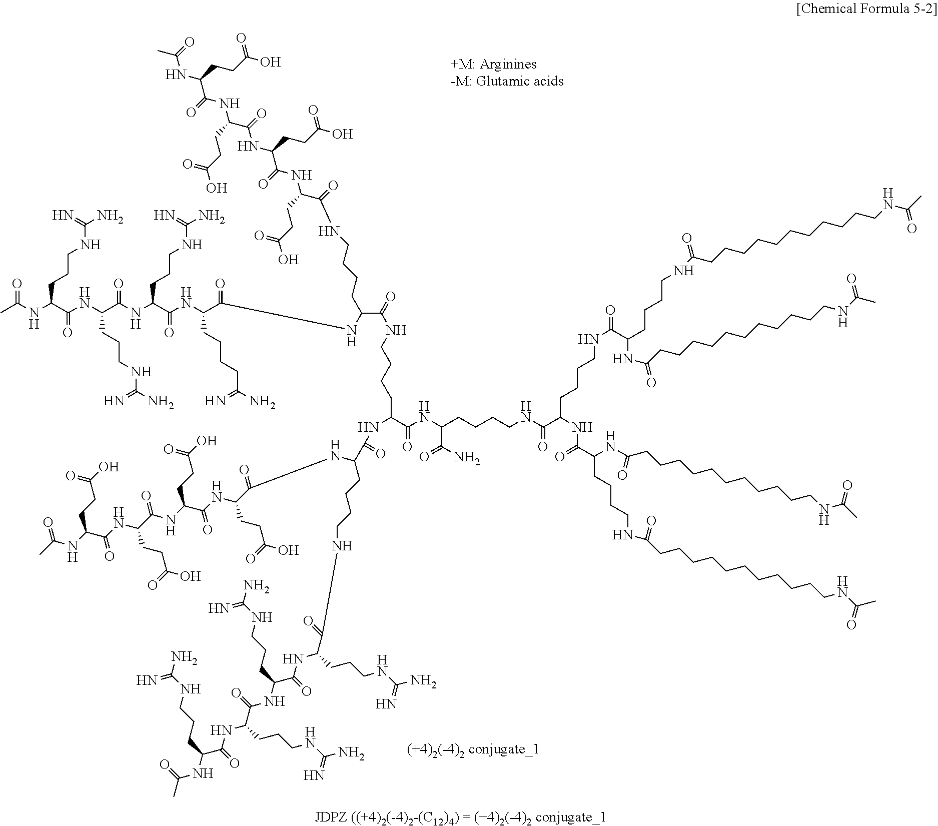

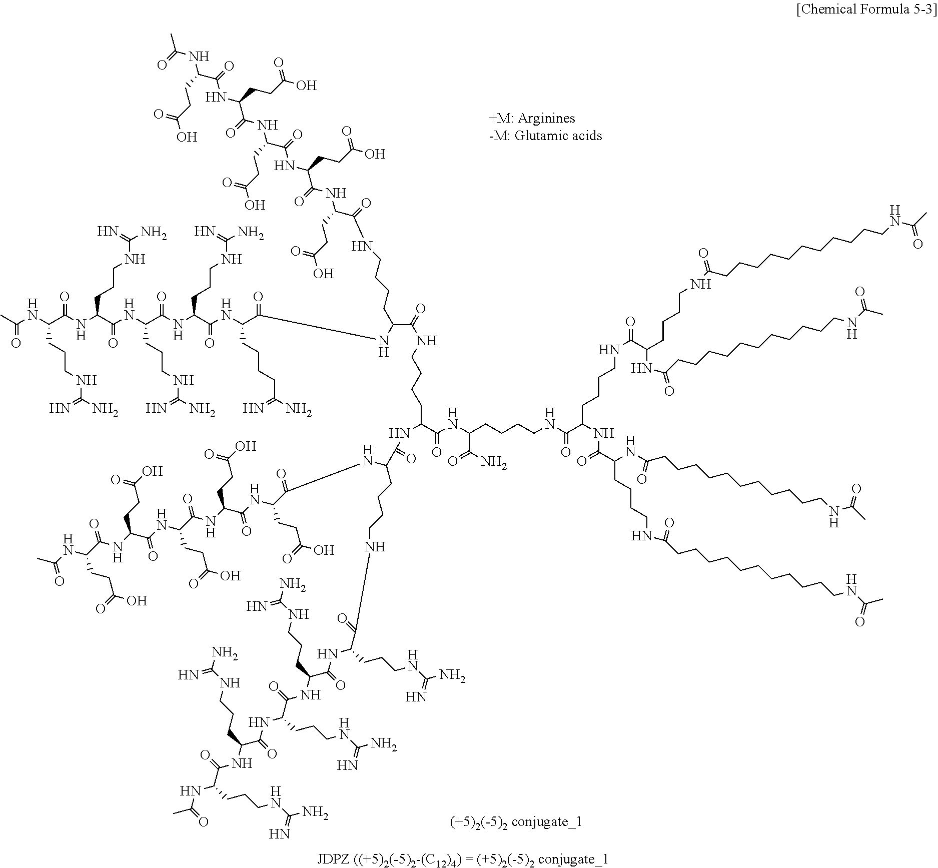

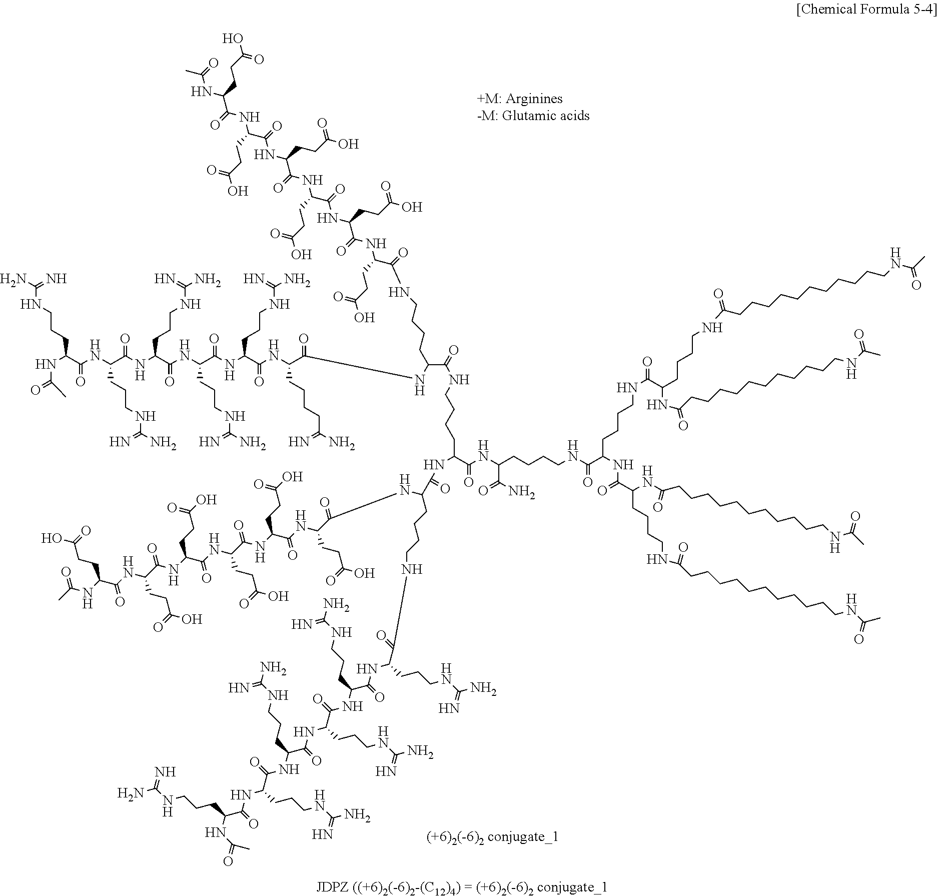

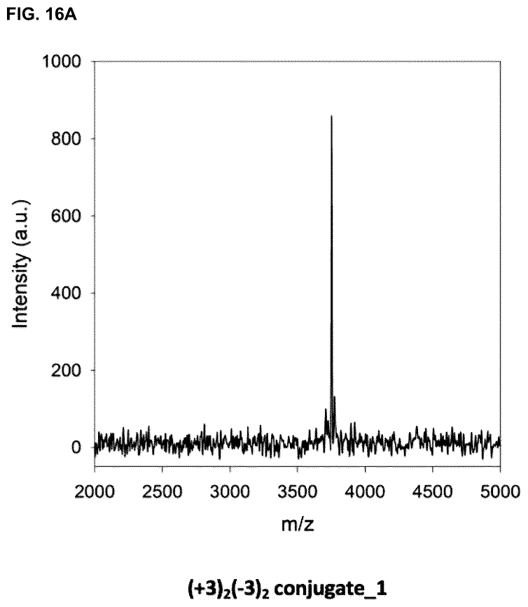

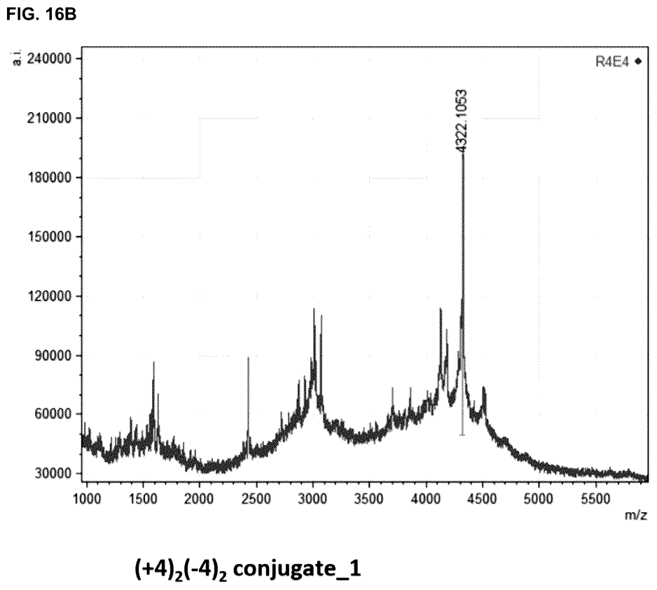

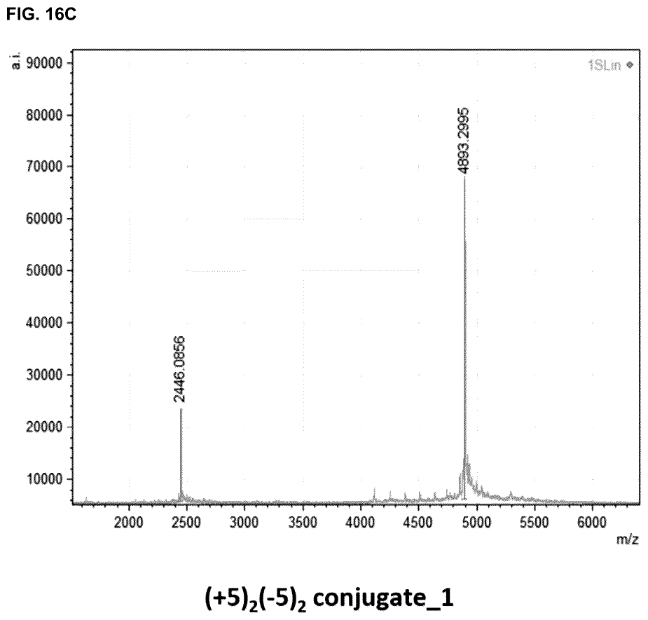

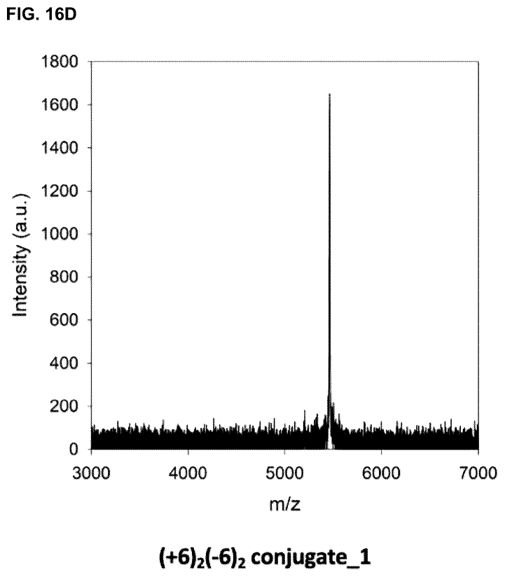

The JDPZ ((+M)₂(−M)₂-(Cₒ)ₚ) (wherein M is the number of amino acid residues constituting the positively charged peptide and the negatively charged peptide, n=2 is the number of strands of peptide, o is the number of carbons, and p is the number of fatty acid alkyl chains, with M being an integer from 1 to 30, o being an integer from 10 to 30, and p being an integer from 1 to 10) prepared through the above-described process was purified by high-performance liquid chromatography (HPLC) at room temperature using a C4 reversed-phase column (Waters, USA). Distilled water (0.1% TFA) and acetonitrile (ACN 0-100%) were used as an eluent. The molecular weight of the molecule was measured using a matrix-assisted laser desorption/ionization time-of-flight (MALDI-TOF) mass spectrometer and the result is shown in FIG. 16. As a result of the analytical HPLC, purity was confirmed to be >95%.

### Examples 6-1 to 6-2. Fluorescent material-introduced G2 Janus-type dendritic polyelectrolyte zwitterion (JDPZ ((+M)₂(−M)₂-(Cₒ)ₚ_FAM)=(+M)₂(−M)₂ conjugate_FAM)

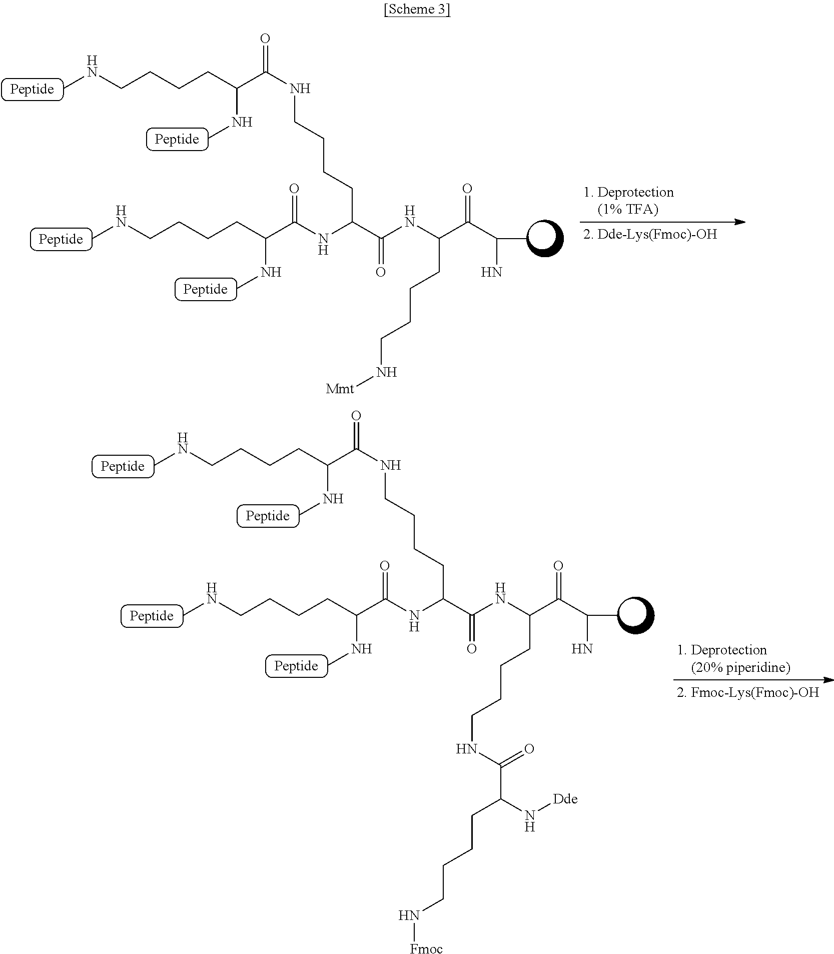

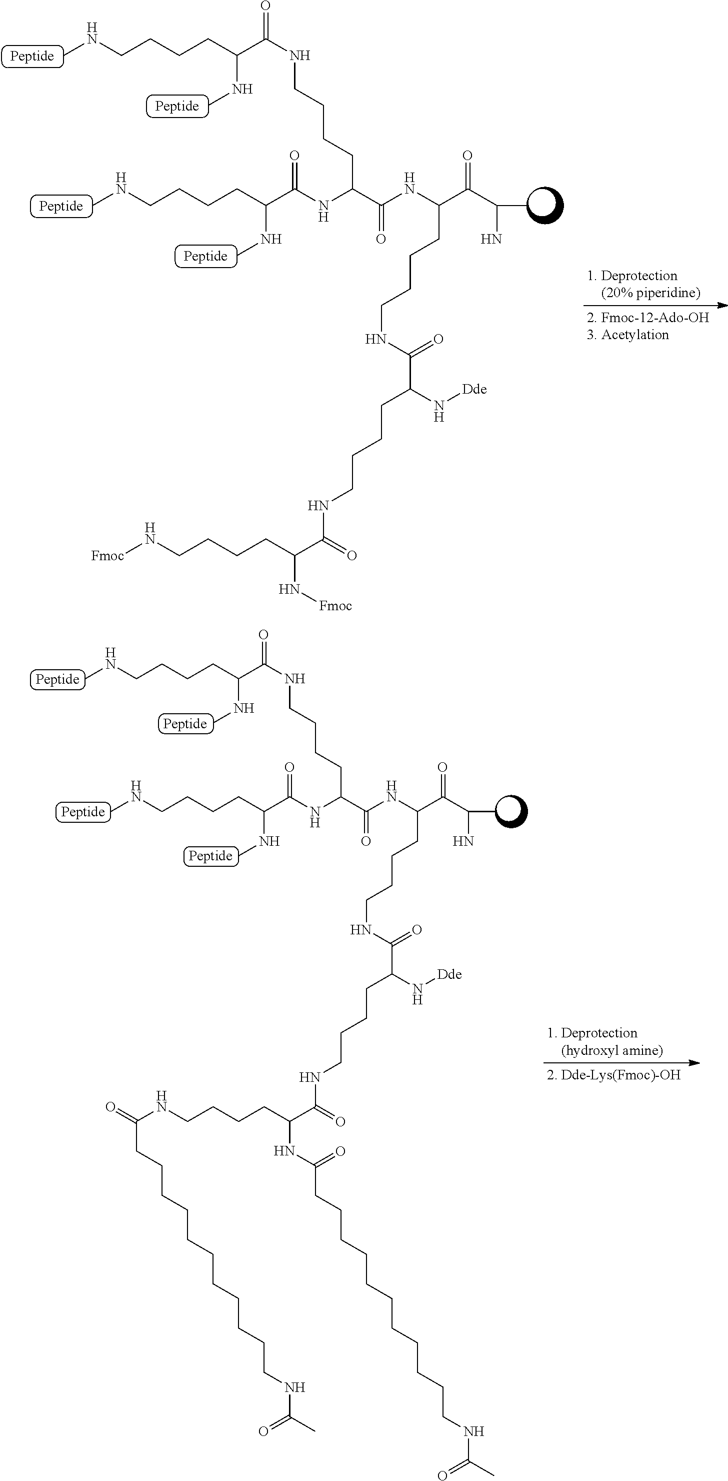

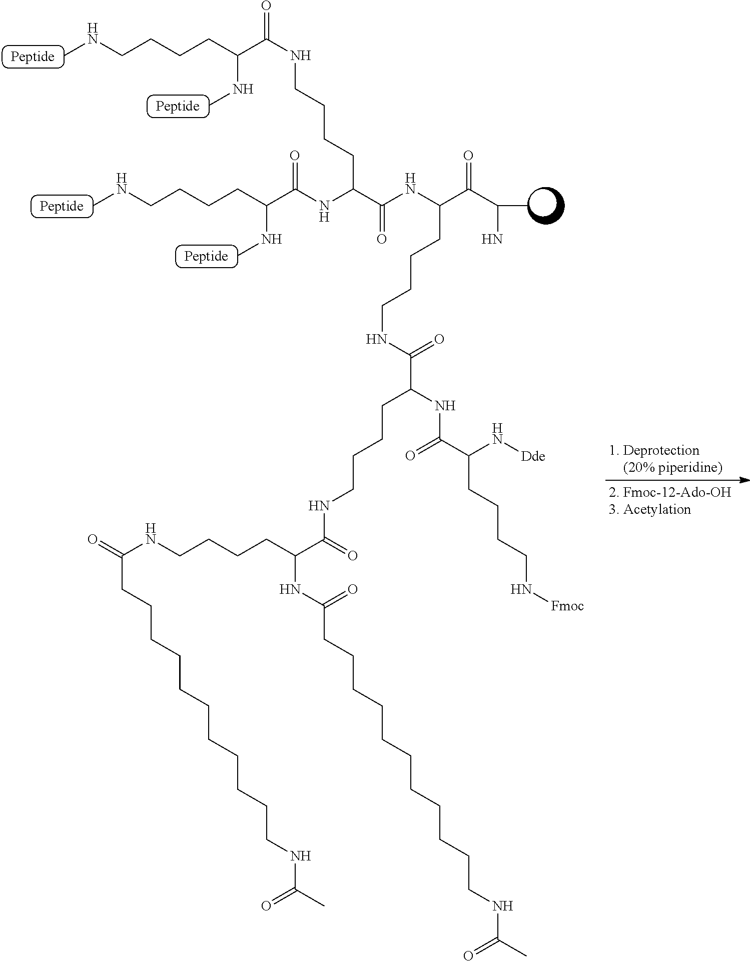

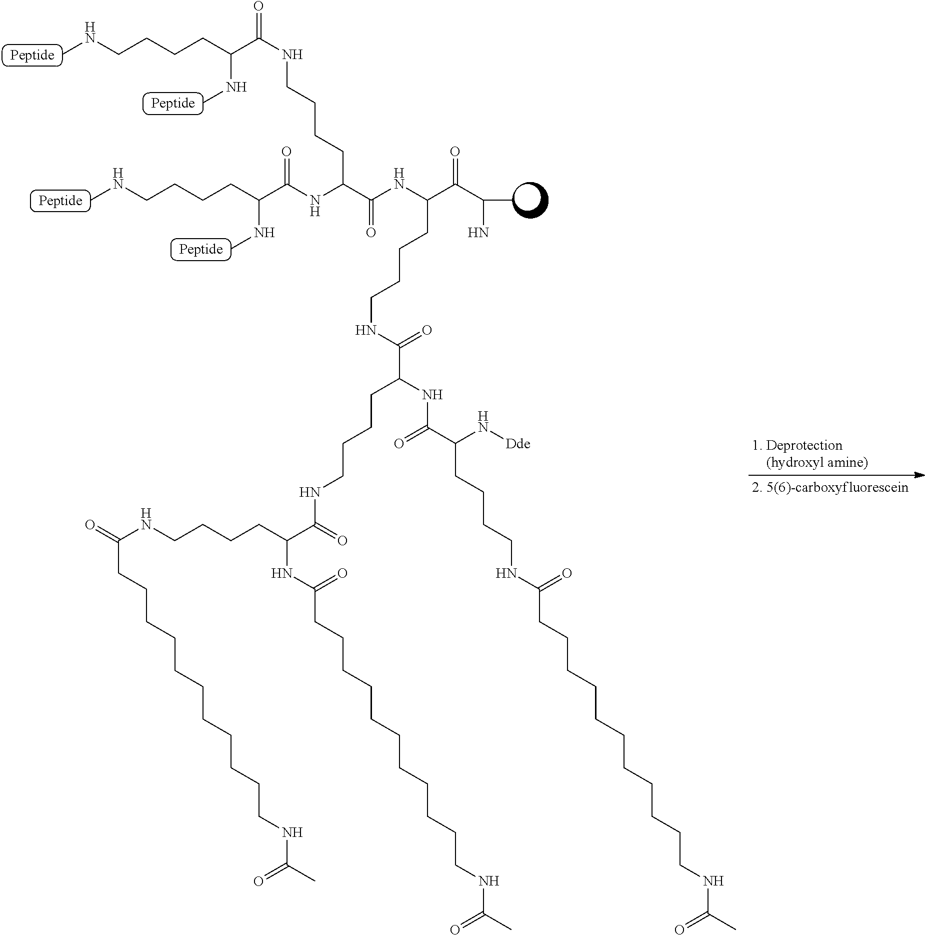

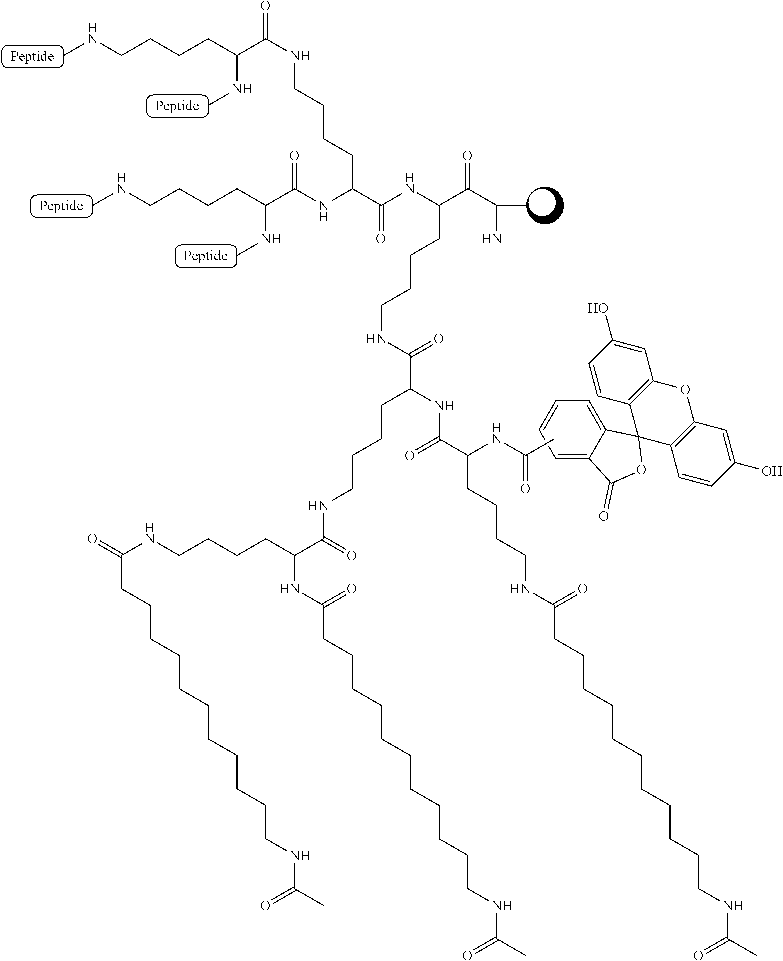

As shown in Scheme 3, after the growth of peptide chain was completed, Dde-Lys(Fmoc)-OH was coupled to the amine generated from Mmt deprotection. Afterwards, Fmoc was deprotected selectively and Fmoc-Lys(Fmoc)-OH was coupled to the resulting amine. After the deprotection of two Fmoc groups with piperidine, two fatty acid alkyl chains were coupled to each amine group. After deprotecting the Dde group, Dde-Lys(Fmoc)-OH was coupled, and sequentially, after deprotection of the Fmoc group, coupling of one fatty acid alkyl chain was performed and after deprotection of the Dde group, coupling of 5(6)-FAM was performed to complete synthesis. Through the above-described process, a Janus-type dendritic polyelectrolyte zwitterion (JDPZ ((+M)₂(−M)₂-(Cₒ)ₚ_FAM) was synthesized (Chemical Formulas 6-1 to 6-2 in order). Since bonds can be formed to various hydrophobic compounds through deprotection using the Mmt group, it can be seen that the hydrophobic compounds applicable to the present disclosure are not particularly limited as long as they can form a bond with an amino group.

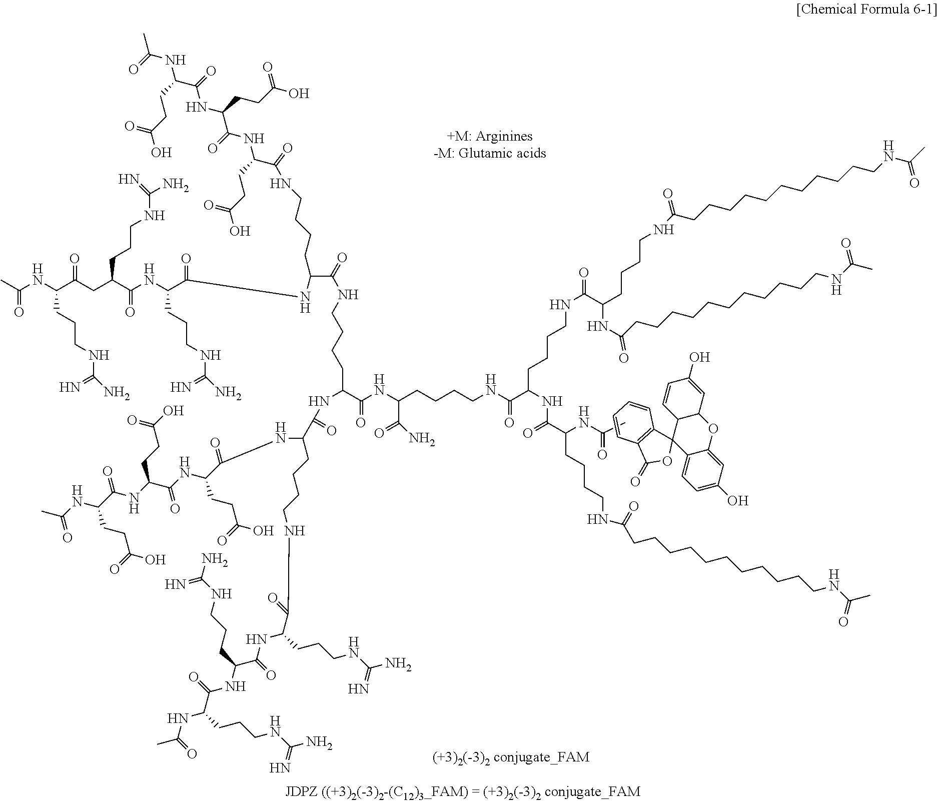

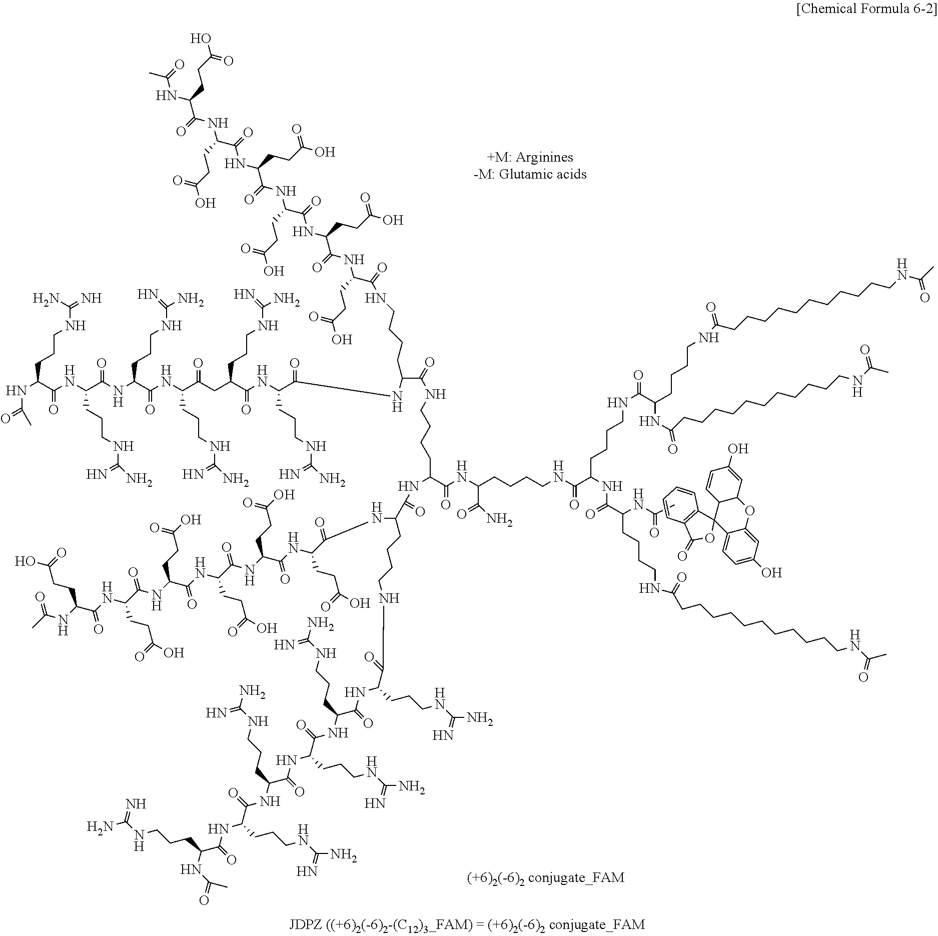

### Example 7. G2 Janus-type dendritic polyelectrolyte zwitterion (JDPZ ((+6)₂(−6)₂-(Cₒ)ₚ(C_{q})ᵣ)═(+6)₂(−6)₂ conjugate_2)

A 2nd generation (G2) polylysine dendrimer was synthesized using the bifurcation synthesis method. Afterwards, two positively charged peptide strands and two negatively charged peptide strands were synthesized sequentially. Then, Fmoc-Lys(Fmoc)-OH was coupled to the amine generated from Mmt deprotection. After removing the Fmoc group with 20% piperidine, two fatty acid alkyl chains were coupled to synthesize a G2 Janus-type dendritic polyelectrolyte zwitterion JDPZ ((+M)₂(−M)₂-(Cₒ)ₚ)═(+M)₂(−M)₂ conjugate 2).

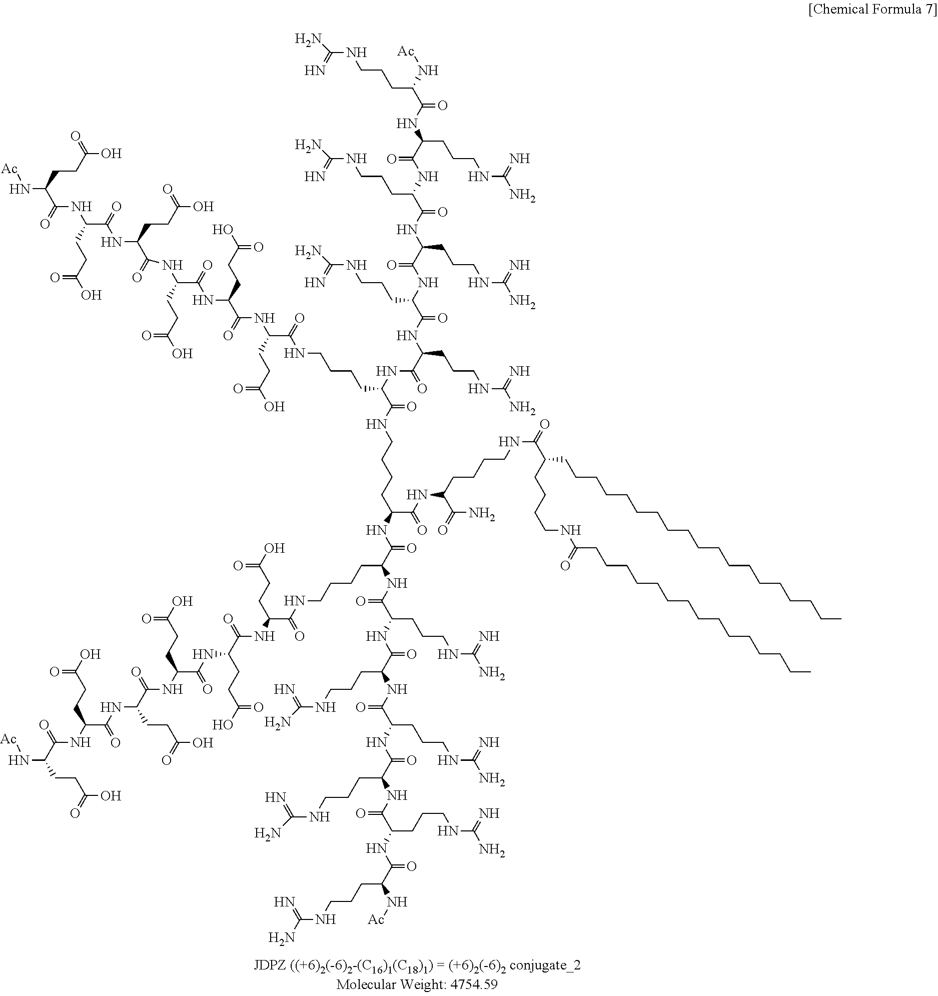

### Example 8. G2 Janus-type dendritic polyelectrolyte zwitterion (JDPZ ((+6)₂(−6)₂-(Cₒ)ₚ(DMG)_{q})=(+6)₂(−6)₂ conjugate 3)

A 2nd generation (G2) polylysine dendrimer was synthesized using the bifurcation synthesis method. Afterwards, two positively charged peptide strands and two negatively charged peptide strands were synthesized sequentially. Then, Fmoc-Lys(Fmoc)-OH was coupled to the amine generated from Mmt deprotection. After the Fmoc group was removed with 20% piperidine, two Dde-Lys(Fmoc)-OH were coupled. After deprotecting the Fmoc group, two DMG (N,N-dimethylglycine) were coupled, and then after deprotecting the Dde group, two Dde-Lys(Fmoc)-OH were coupled. Next, after deprotecting the Fmoc group, two fatty acid alkyl chains were coupled, and then after deprotecting the Dde group, two DMGs were coupled to synthesize a G2 Janus-type dendritic polyelectrolyte zwitterion JDPZ ((+6)₂(−6)₂-(Cₒ)ₚ(DMG)_{q})=(+6)₂(−6)₂ conjugate_3).

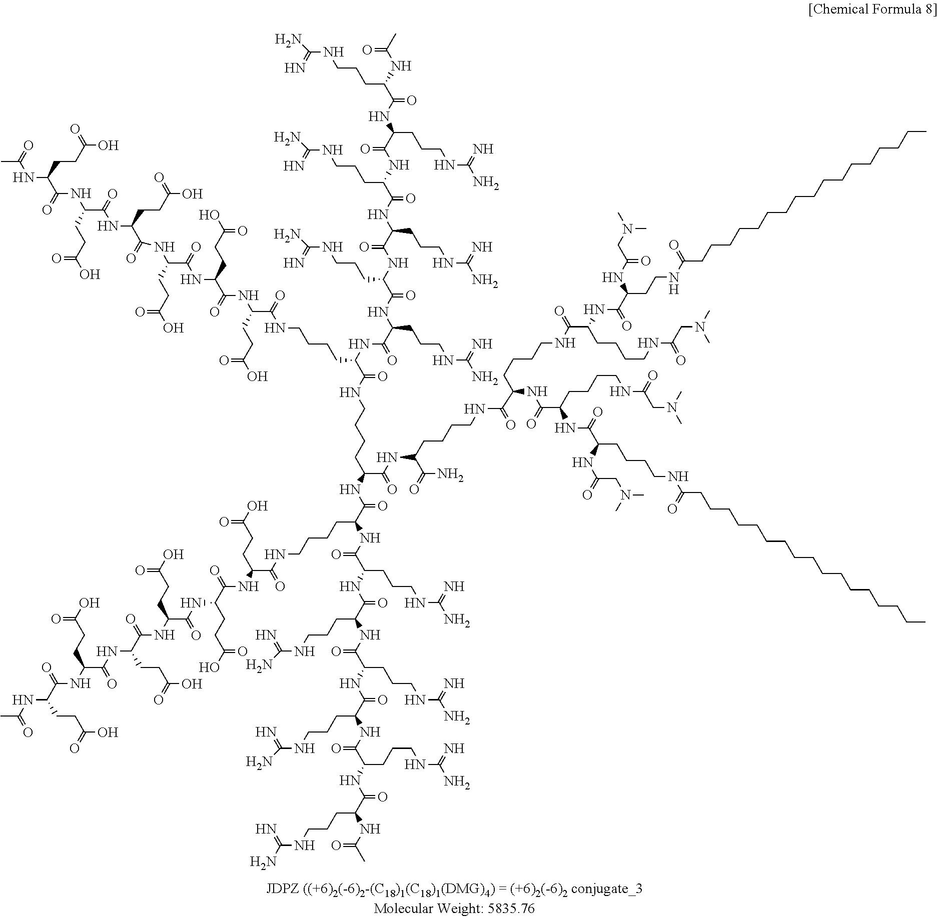

### Comparative Examples 1-1 to 1-3. G2 Janus-type dendrimer

A G2 Janus-type dendrimer was synthesized in the same manner as in Example 5-1, except that arginine was used as the positively charged peptide, glutamic acid was used as the negatively charged peptide, and serine (Ser, S) was used as the neutral charge (Ø) (Comparative Examples 1-1 to 1-3 in order).

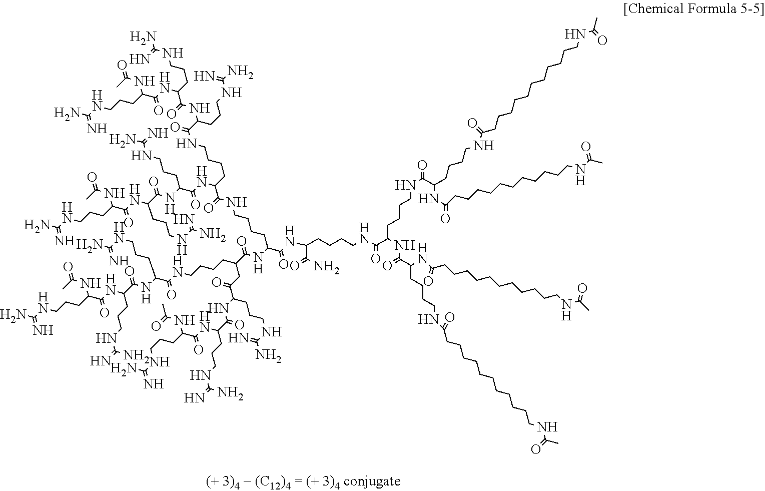 [SMILES: CC(=O)NCCCCCCCCCCCC(=O)NCCCCC(NC(=O)CCCCCCCCCCCNC(C)=O)C(=O)NCCCCC(NC(=O)C(CCCCNC(=O)CCCCCCCCCCCNC(C)=O)NC(=O)CCCCCCCCCCCNC(C)=O)C(=O)NCCCCC(NC(=O)C(CCCCNC(=O)C(CCCCNC(=O)C(CCCNC(=N)N)NC(=O)C(CCCNC(=N)N)NC(=O)C(CCCNC(=N)N)NC(C)=O)NC(=O)C(CCCNC(=N)N)NC(=O)C(CCCNC(=N)N)NC(=O)C(CCCNC(=N)N)NC(C)=O)NC(=O)C(CCCCNC(=O)C(CCCNC(=N)N)NC(=O)C(CCCNC(=N)N)NC(=O)C(CCCNC(=N)N)NC(C)=O)CC(=O)C(CCCNC(=N)N)NC(=O)C(CCCNC(=N)N)NC(=O)C(CCCNC(=N)N)NC(C)=O)C(N)=O]

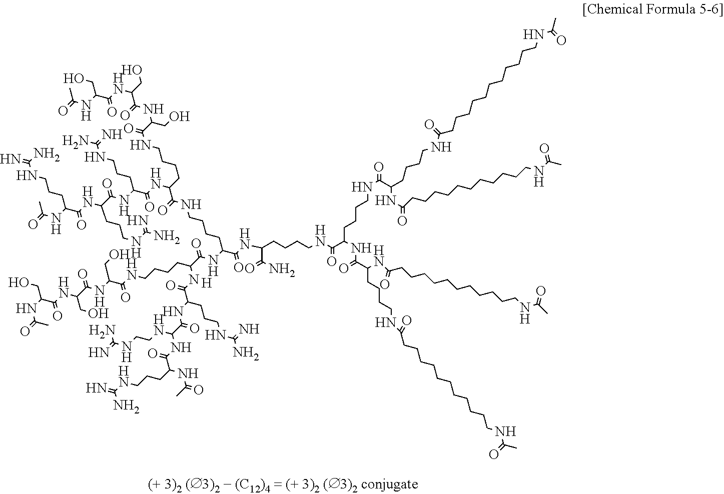 [SMILES: CC(=O)NCCCCCCCCCCCC(=O)NCCCCC(NC(=O)CCCCCCCCCCCNC(C)=O)C(=O)NCCCCC(NC(=O)C(CCCCNC(=O)CCCCCCCCCCCNC(C)=O)NC(=O)CCCCCCCCCCCNC(C)=O)C(=O)NCCCCC(NC(=O)C(CCCCNC(=O)C(CCCCNC(=O)C(CO)NC(=O)C(CO)NC(=O)C(CO)NC(C)=O)NC(=O)C(CCCNC(=N)N)NC(=O)C(CCCNC(=N)N)NC(=O)C(CCCNC(=N)N)NC(C)=O)NC(=O)C(CCCCNC(=O)C(CO)NC(=O)C(CO)NC(=O)C(CO)NC(C)=O)NC(=O)C(CCCNC(=N)N)NC(=O)C(NCCNC(=N)N)NC(=O)C(CCCNC(=N)N)NC(C)=O)C(N)=O]

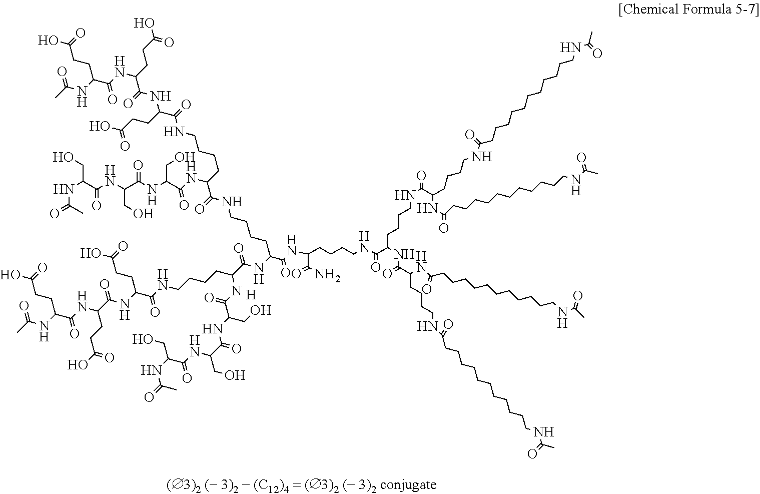 [SMILES: CC(=O)NCCCCCCCCCCCC(=O)NCCCCC(NC(=O)CCCCCCCCCCCNC(C)=O)C(=O)NCCCCC(NC(=O)C(CCCCNC(=O)CCCCCCCCCCCNC(C)=O)NC(=O)CCCCCCCCCCCNC(C)=O)C(=O)NCCCCC(NC(=O)C(CCCCNC(=O)C(CCCCNC(=O)C(CCC(=O)O)NC(=O)C(CCC(=O)O)NC(=O)C(CCC(=O)O)NC(C)=O)NC(=O)C(CO)NC(=O)C(CO)NC(=O)C(CO)NC(C)=O)NC(=O)C(CCCCNC(=O)C(CCC(=O)O)NC(=O)C(CCC(=O)O)NC(=O)C(CCC(=O)O)NC(C)=O)NC(=O)C(CO)NC(=O)C(CO)NC(=O)C(CO)NC(C)=O)C(N)=O]

### Comparative Examples 2-1 to 2-2. Fluorescent material-introduced G2 Janus-type dendrimer

A G2 Janus-type dendrimer was synthesized in the same manner as in Example 5-1, except that arginine was used as the positively charged peptide, glutamic acid was used as the negatively charged peptide, and serine (Ser, S) was used as the neutral charge (Ø) (Comparative Examples 2-1 to 2-2 in order).

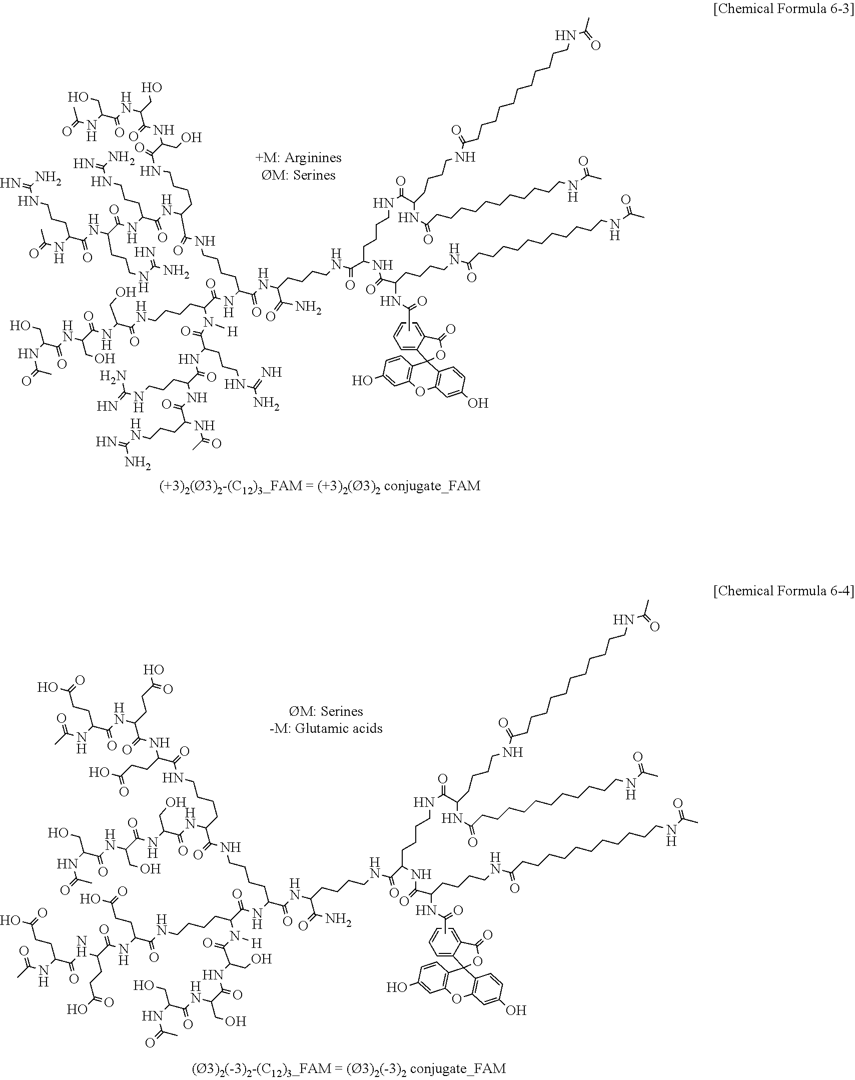

### Comparative Examples 3-1 to 3-2. Zwitterionic peptide complex (Ac(ER)12, AcE12R12)

A peptide represented by SEQ TD NO: 163 (3-1), in which cationic and anionic amino acids are linked alternately, and a peptide represented by SEQ ID NO: 164 (3-2), in which zwitterionic peptides and anionic peptides are bound, were prepared by solid-phase peptide synthesis (SPSS).

### Experimental Example 1. Characterization of Dendritic Polyelectrolyte Zwitterion (DPZ)

### 1) MALDI-TOF mass spectrometry

The synthesis of the dendritic polyelectrolyte zwitterions (DPZs) prepared in Examples 1-1 to 1-6 was analyzed by MALDI-TOF.

FIG. 2a is the MALDI-TOF spectrum of (+1)₂(−1)₂ prepared in Example 1-1, FIG. 2b is the MALDI-TOF spectrum of (+2)₂(−2)₂ prepared in Example 1-2, FIG. 2c is the MALDI-TOF spectrum of (+3)₂(−3)₂ prepared in Example 1-3, FIG. 2d is the MALDI-TOF spectrum of (+4)₂(−4)₂ prepared in Example 1-4, FIG. 2e is the MALDI-TOF spectrum of (+5)₂(−5)₂ prepared in Example 1-5, and FIG. 2f is the MALDI-TOF spectrum (+6)₂(−6)₂ prepared in Example 1-6. It can be seen that the dendritic polyelectrolyte zwitterions (DPZs) prepared in Examples 1-1 to 1-6 were synthesized successfully.

### 2) Cytotoxicity

Since DPZ contains both polyelectrolytes and zwitterions in a single molecule, it is expected to have non-fouling property and low toxicity, while also having advantageous intracellular delivery efficiency due to its interaction with the cell membrane. Cytotoxicity was analyzed first. HeLa cells were subcultured in DMEM supplemented with 10% FBS (fetal bovine serum), dispensed into each well at 1×10⁴ cells/well, and cultured at 37° C. for 24 hours. The cells were treated with the samples (Examples 1-1 to 1-6 or Comparative Examples 3-1 and 3-2) at various concentrations and then cultured at 37° C. for 4 hours. WST-8 solution was added to the cultured cells, and absorbance was measured at 450 nm with a microplate reader (Perkin-Elmer, USA) 4 hours later.

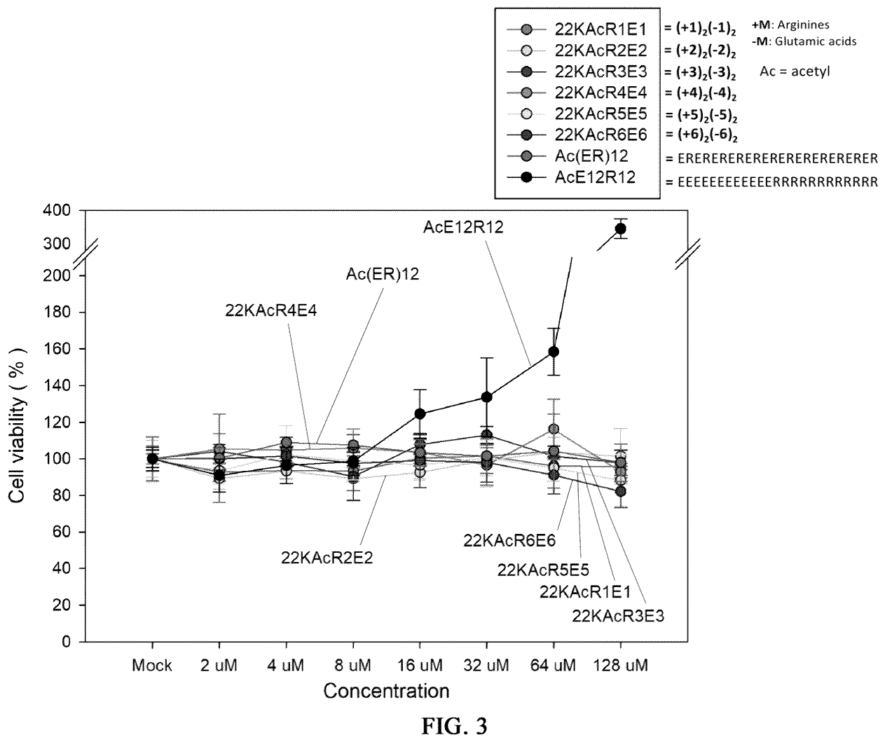

FIG. 3 shows the result of analyzing the cytotoxicity of the dendritic polyelectrolyte zwitterions prepared in Examples 1-1 to 1-6 (DPZ(+M)ₙ(−M)ₙ) and the peptides prepared in Comparative Examples 3-1 and 3-2 for the HeLa cells. As shown in FIG. 3, it can be seen that the zwitterion of Comparative Example 3-1, the polyelectrolyte zwitterion of Comparative Example 3-2, and the dendritic polyelectrolyte zwitterions (DPZ(+M)ₙ(−M)ₙ) of Examples 1-1 to 1-6 are stable substances that do not cause toxicity in the cells. It was confirmed that the polyelectrolyte-zwitterion of Comparative Example 3-2 promoted cell growth.

### 3) Characterization of Non-Fouling Property

Various proteins and genetic materials exist in living organisms. It is known that conventional cell-penetrating peptides or delivery systems cannot deliver drugs effectively or cause side effects due to interactions with various molecules in the body. The non-fouling property of the dendritic polyelectrolyte zwitterions according to the present disclosure for molecules in the body was investigated by EMSA (electrophoretic mobility shift assay).

First, 0.5 μg of BSA, an adhesive protein present in the blood at a high proportion, was prepared as a model protein and mixed with the dendritic polyelectrolyte zwitterions of Examples 1-1 to 1-6 (DPZ(+M)ₙ(−M)ₙ) at various concentration ratios. The mixing ratio was R/B ratio, wherein the R/B ratio was arginine/BSA ratio. After reacting the mixture at room temperature for 12 hours, electrophoresis was performed on a 12% native polyacrylamide gel electrophoresis (PAGE) gel at 22 mA for 90 minutes. For visualization, the bands were stained with Coomassie brilliant blue R-250.

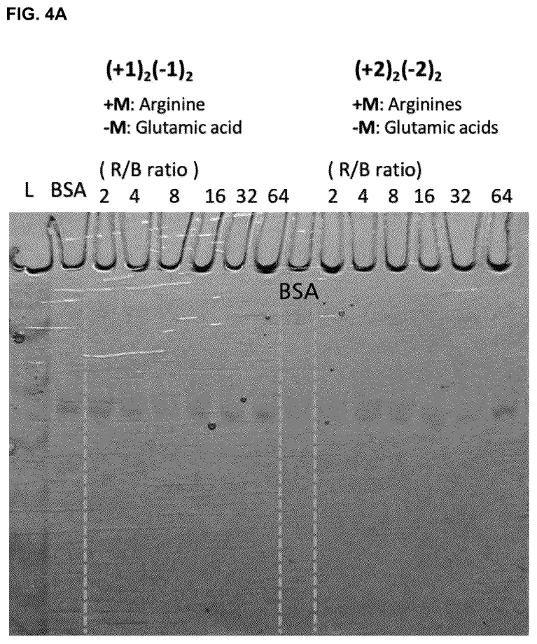

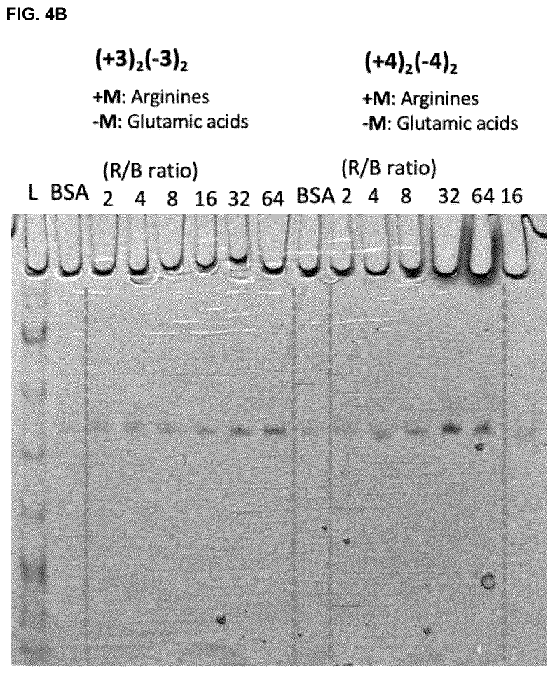

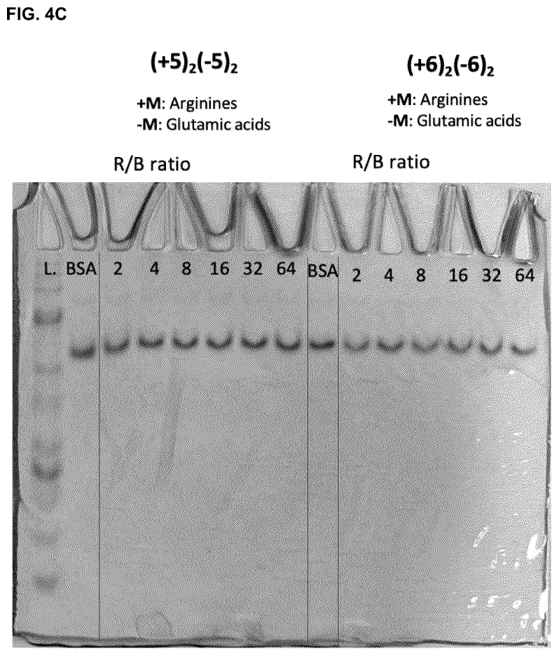

FIG. 4a shows the EMSA result of analyzing the non-fouling property of the dendritic polyelectrolyte zwitterions of Examples 1-1 and 1-2 for proteins ((+1)₂(−1)₂ and (+2)₂(−2)₂). FIG. 4b shows an EMSA result of analyzing the non-fouling property of dendritic polyelectrolyte zwitterions of Examples 1-3 and 1-4 ((+3)₂(−3)₂ and (+4)₂(−4)₂) for proteins. FIG. 4c shows an EMSA result of analyzing the non-fouling property of dendritic polyelectrolyte zwitterions of Examples 1-5 and 1-6 ((+5)₂(−5)₂ and (+6)₂(−6)₂) for proteins. In FIGS. 4a to 4c, ‘L’ is a protein marker, and R/B ratio is the mixing ratio of arginine present in the DPZ and BSA.

As shown in FIGS. 4a to 4c, the dendritic polyelectrolyte zwitterions of Examples 1-1 to 1-6 did not bind to BSA even in a mixed solution in which the dendritic polyelectrolyte zwitterion is in excess of BSA (R/B ratio>1). That is to say, it can be seen that the dendritic polyelectrolyte zwitterions of Examples 1-1 to 1-6 have significantly higher non-fouling property with respect to proteins existing in vivo.

### 4) Comparative Analysis of Non-Fouling Property of Linear R12 with the Same Number of Cationic Residues as Example 1-6

A linear peptide R12, which has the same number of cationic residues (12 arginines) as the dendritic polyelectrolyte zwitterion of Example 1-6 ((+6)₂(−6)₂), was prepared by solid-phase peptide synthesis (SPSS) and was analyzed by EMSA in the same way as in ‘3) Characterization of non-fouling property’.

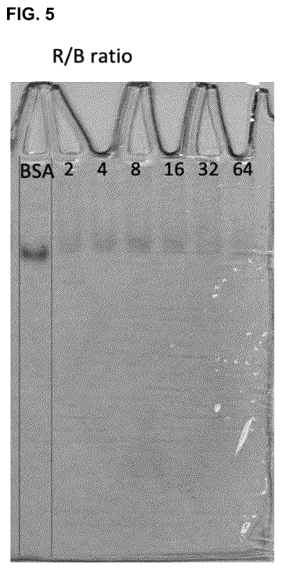

FIG. 5 shows an EMSA result of analyzing the non-fouling property of a linear peptide R12 (SEQ ID NO: 165) for proteins. It was confirmed that the linear peptide R12 (SEQ ID NO: 165) interacts with BSA and induces EMS (electrophoretic mobility shift) and the weakening of the intensity of the BSA band. Therefore, the linear peptide R12 (SEQ ID NO: 165), which has only cationic residues, did not exhibit non-fouling property.

### Experimental Example 3. Formation, Cellular Delivery and Expression of mRNA Complex

It was confirmed that the dendritic polyelectrolyte zwitterion (DPZ) of the present disclosure exists stably inside cells without toxicity. Next, it was investigated whether the dendritic polyelectrolyte zwitterion according to the present disclosure plays a role as a carrier of a genetic material and as a therapeutic agent through fusion with a genetic material.

### 1) Analysis of Complex Formation Between Dendritic Polyelectrolyte Zwitterion and Genetic Material

Single-stranded DNA (ssDNA) (SEQ ID NO: 166) and mRNA (SEQ ID NO: 167) were purchased from Bioneer (Korea) and TriLink BioTechnologies (USA), respectively, as genetic materials. Plasmid DNA (SEQ ID NO. 168) with a double helix structure was prepared by cloning in-house. In the case of the mRNA, although the total length of the sequence is 997 nt, since the manufacturer (Trilink BioTechnologies) discloses only the open reading frame (ORF) 720 nt, only the corresponding sequence was described.

Complexes were prepared by varying the mixing ratio of each genetic material and the dendritic polyelectrolyte zwitterion of Examples 1-6 ((+6)₂(−6)₂). The mixing ratio is expressed as charge ratio (+/−), which is calculated by (total positive charge of DPZ)/(total negative charge of genetic material). The mixing amount of the DPZ of Examples 1-6 was calculated based on the weight of the genetic materials (ssDNA 200 nt, mRNA 997 nt, plasmid DNA 4,848 bp). DW was used as a mixing solvent. The mixture was subjected to electrophoresis on a 0.8% agarose gel at 100 V for 30 minutes to confirm the formation of a complex.

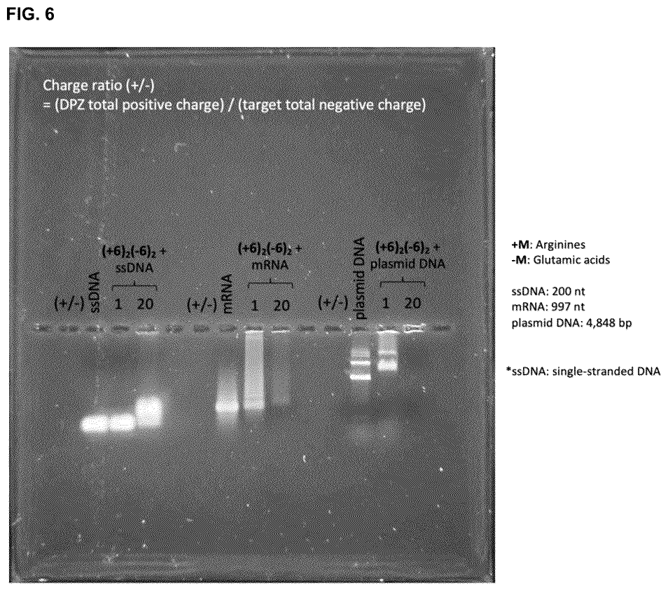

FIG. 6 shows an EMSA result of analyzing the interaction pattern between a dendritic polyelectrolyte zwitterion of Examples 1-6 ((+6)₂(−6)₂) and various genetic materials (single-stranded DNA, mRNA, and plasmid DNA).

As shown in FIG. 6, the dendritic polyelectrolyte zwitterion of Examples 1-6 ((+6)₂(−6)₂) formed complexes with various types of genetic materials such as single-stranded DNA, mRNA and plasmid DNA, confirming that it induces the EMS (electrophoretic mobility shift) of these genetic materials.

### 2) Formation of Complex Between Dendritic Polyelectrolyte Zwitterion and mRNA

Complexes of mRNA and the dendritic polyelectrolyte zwitterions (DPZ(+M)ₙ(−M)ₙ of Examples 1-3 to 1-6) were prepared with different charge ratios. Specifically, the amount of EGFP mRNA was fixed to 997 nt, and the dendritic polyelectrolyte zwitterions of Examples 1-3 to 1-6 ((+3)₂(−3)₂, (+4)₂(−4)₂, (+5)₂(−5)₂ and (+6)₂(−6)₂) were mixed at a charge ratio of 0 to 4. The mixing ratio is expressed as charge ratio (+/−), which is calculated by (total positive charge of DPZ)/(total negative charge of genetic material). The mixture was subjected to electrophoresis on a 0.8% agarose gel at 100 V for 30 minutes to confirm complex formation.

FIG. 7 shows an EMSA result of analyzing complex formation between dendritic polyelectrolyte zwitterions of Examples 1-3 to 1-6 ((+3)₂(−3)₂, (+4)₂(−4)₂, (+5)₂(−5)₂ and (+6)₂(−6)₂) and mRNA depending on charge ratio.

As shown in FIG. 7, the dendritic polyelectrolyte zwitterions of Examples 1-3 to 1-6 induced stronger EMS as the charge ratio (+/−) increased. In addition, it was confirmed that the dendritic polyelectrolyte zwitterion of Examples 1-3 to 1-6 induced stronger EMS as the length of the positively charged peptide and the negatively charged peptide increased.

### Experimental Example 4. Characterization of Fluorescent Material-Introduced Dendritic Polyelectrolyte Zwitterion (DPZ)

### 1) MALDI-TOF Mass Spectrometry

The synthesis of DPZ ((+M)ₙ(−M)ₙ_FAM) into which the fluorescent material FAM was introduced, prepared in Examples 2-1 to 2-6, was confirmed by MALDI-TOF analysis.

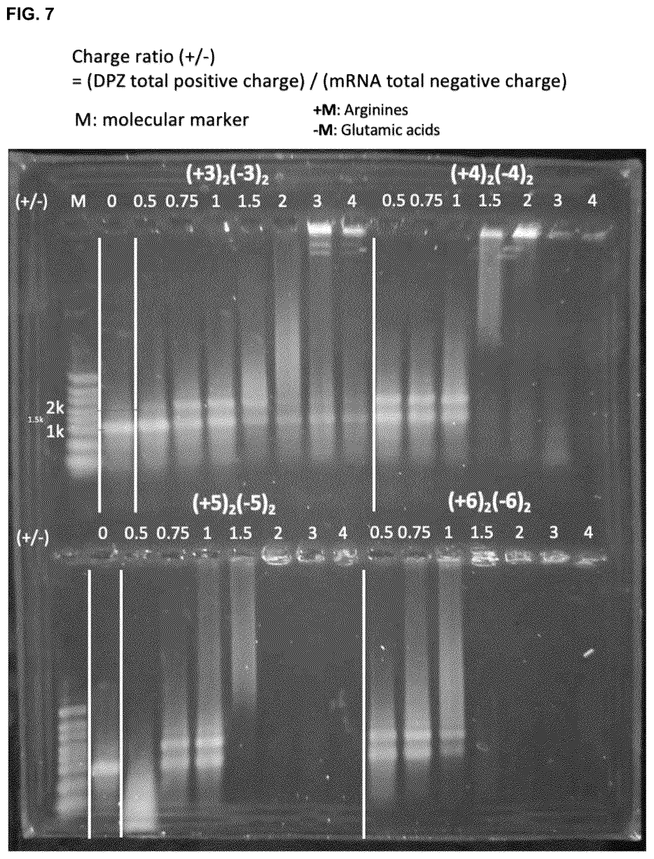

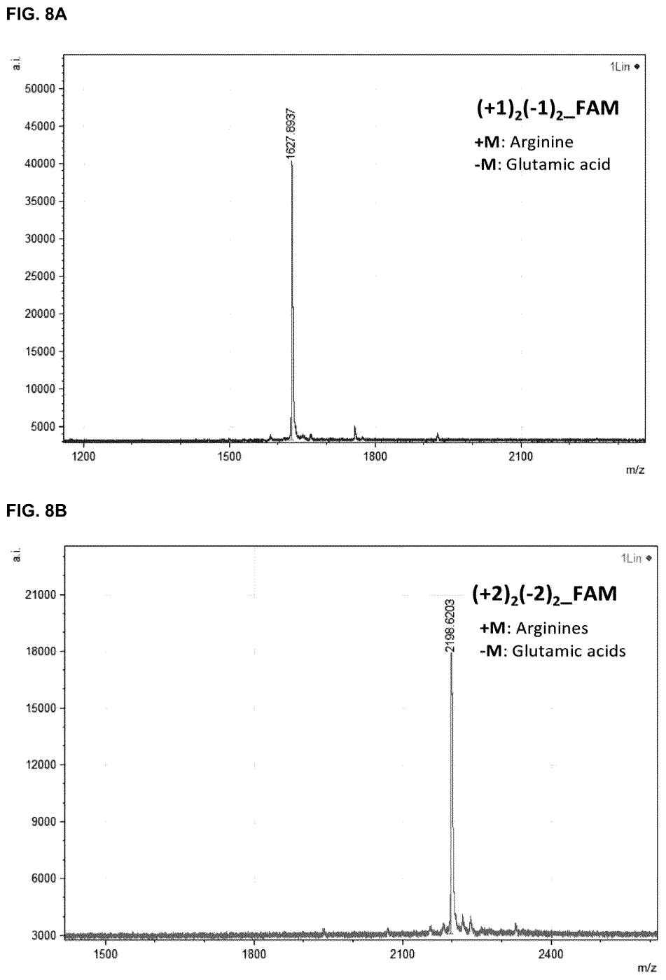

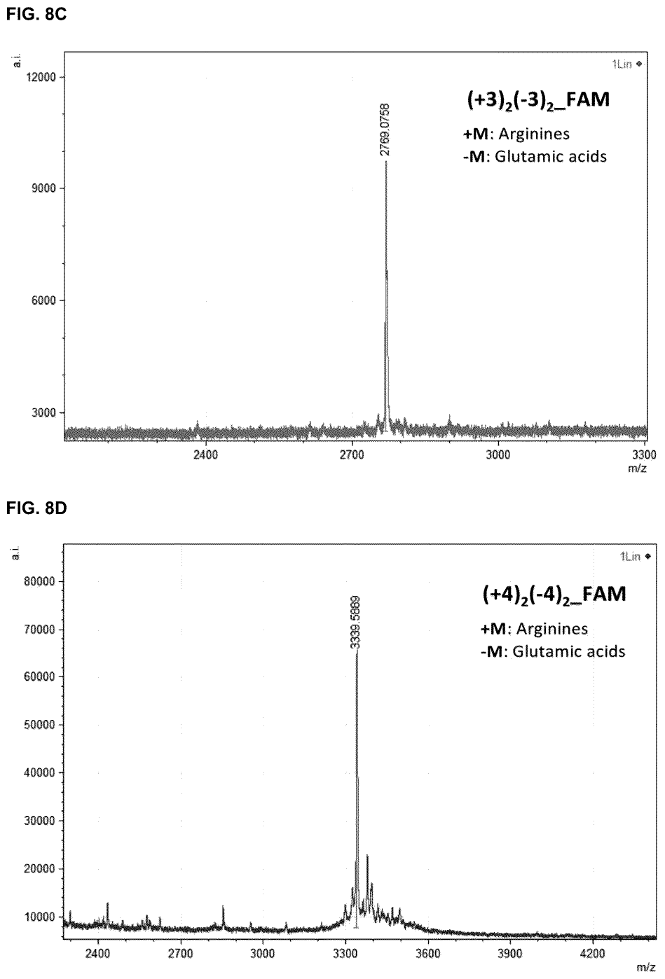

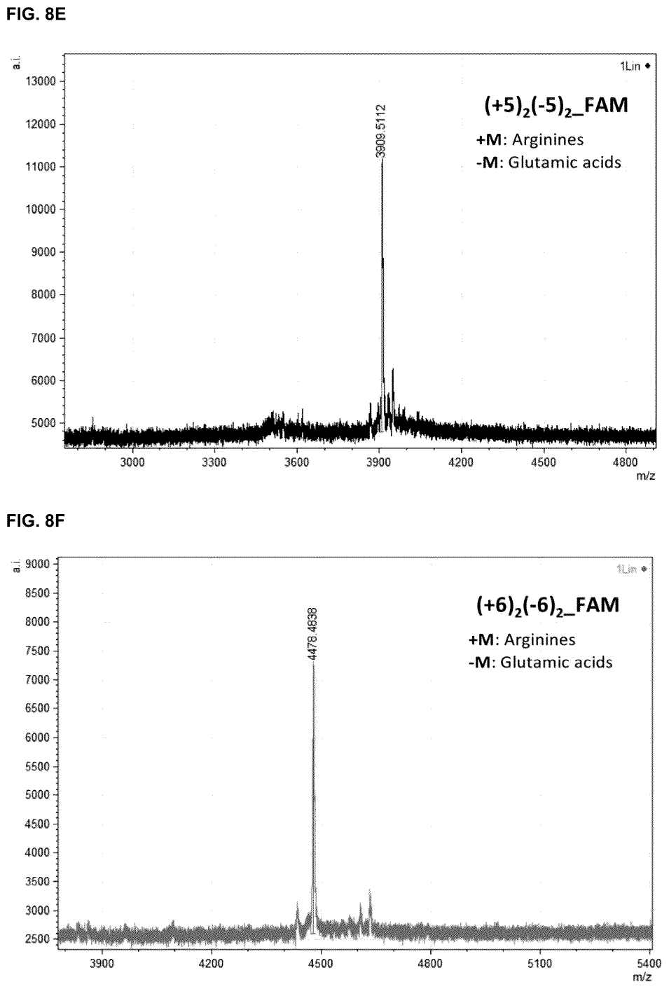

FIGS. 8a to 8f show the MALDI-TOF spectra of DPZ(+M)ₙ(−M)ₙ_FAM) into which the fluorescent material FAM was introduced, prepared in Examples 2-1 to 2-6. FIG. 8a: (+1)₂(−1)₂_FAM of Example 2-1, FIG. 8b: (+2)₂(−2)₂_FAM of Example 2-2, FIG. 8c: (+3)₂(−3)₂_FAM of Example 2−3, FIG. 8d: (+4)₂(−4)₂_FAM of Example 2-4, FIG. 8e: (+5)₂(−5)₂_FAM of Example 2-5, FIG. 8f: (+6)₂(−6)₂_FAM of Example 2-6.

As shown in FIGS. 8a to 8f, it was confirmed that the dendritic polyelectrolyte zwitterions ((+M)ₙ(−M)ₙ_FAM) into which the fluorescent material was introduced (Examples 2-1 to 2-6) were synthesized successfully.

### 2) Intracellular Delivery Efficiency

It was investigated whether the DPZs ((+M)ₙ(−M)ₙ_FAM) into which the fluorescent material FAM was introduced, prepared in Examples 2-1 to 2-6, are delivered well into cells. For this purpose, intracellular delivery efficiency was analyzed using HeLa cells. First, HeLa cells were subcultured in DMEM supplemented with 10% FBS (fetal bovine serum), dispensed into each well at 1−3×10⁴ cells/well, and cultured at 37° C. for 24 hours. The cells were treated with (+M)ₙ(−M)ₙ_FAM (Examples 2-1 to 2-6) at a concentration of 16 μM or 32 M, respectively, and incubated at 37° C. for 4 hours. Afterwards, the cells were washed with Dulbecco's phosphate-buffered saline (DPBS), trypsinized, resuspended in DPBS supplemented with 10% cell dissociation buffer (GIBCO) and 1% FBS, and then analyzed using a flow cytometer (BD LSR II SORT flow cytometer, BD Bioscience, USA) for analysis of intracellular fluorescence.

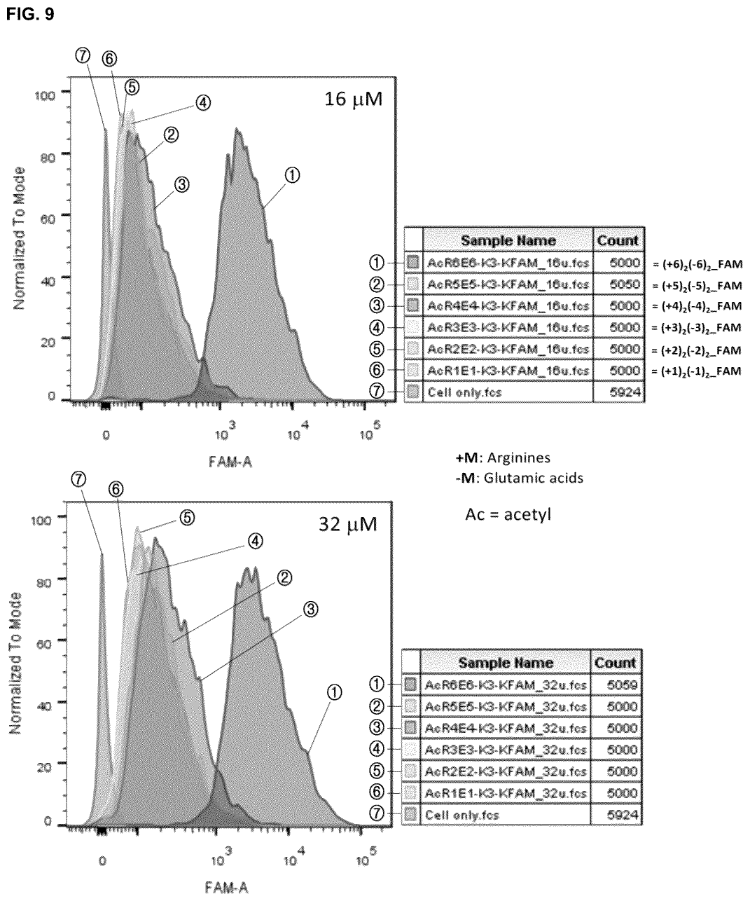

FIG. 9 shows a result of analyzing the intracellular delivery efficiency of the (+M)ₙ(−M)ₙ_FAM of Examples 2-1 to 2-6 in the HeLa cells by flow cytometry (FACS) depending on concentration (16 μM (top), 32 μM (bottom)). As shown in FIG. 9, it was confirmed that all of the dendritic polyelectrolyte zwitterions into which the fluorescent material was introduced of Examples 2-1 to 2-6 had a high intracellular delivery efficiency of 95% or higher.

### Experimental Example 5. Characterization of G1 Janus-Type Dendritic Polyelectrolyte Zwitterion (JDPZ)

### 1) MALDI-TOF Mass Spectrometry

The synthesis of the G1 Janus-type dendritic polyelectrolyte zwitterions prepared in Examples 3-1 to 3-6 (JDPZ, (+M)₁(−M)₁ conjugate_1) was confirmed by MALDI-TOF analysis.

FIGS. 10a to 10f show the MALDI-TOF spectra of G1 Janus-type dendritic polyelectrolyte zwitterions (JDPZ, (+M)₁(−M)₁ conjugate_1) prepared in Examples 3-1 to 3-6. FIG. 10a: (+1)₁(−1)₁ conjugate_1 of Example 3-1, FIG. 10b: (+2)₁(−2)₁ conjugate_1 of Example 3-2, FIG. 10c: (+3)₁(−3)₁ conjugate_1 of Example 3-3, FIG. 10d: (+4)₁(−4)₁ conjugate_1 of Example 3-4, FIG. 10e: (+5)₁(−5)₁ conjugate_1 of Example 3-5, FIG. 10f: (+6)₁(−6)₁ conjugate_1 of Example 3-6.

As shown in FIGS. 10a to 10f, it was confirmed that the G1 Janus-type dendritic polyelectrolyte zwitterions prepared in Examples 3-1 to 3-6 were also synthesized successfully.

### 2) Structural Analysis

Since it was confirmed that the Janus-type dendritic polyelectrolyte zwitterion (JDPZ) according to the present disclosure forms a supramolecular structure through self-assembly in solution, the morphology and structure of the Janus-type dendritic polyelectrolyte zwitterion supramolecular structure according to the present disclosure were investigated.

Each of the G1 Janus-type dendritic polyelectrolyte zwitterions of Examples 3-1 to 3-6 (JDPZs, (+M)₁(−M)₁ conjugate_1)((+1)₁(−1)₁-conjugate_1, (+2)₁(−2)₁-conjugate_1, (+3)₁(−3)₁-conjugate_1, (+4)₁(−4)₁-conjugate_1, (+5)₁(−5)₁-conjugate_1 and (+6)₁(−6)₁-conjugate_1) was mixed in DW and then analyzed by AFM as follows. AFM was performed using the NX10 system (Park Systems, Korea) in non-contact mode. After dissolving each of the samples in DW, 1 μL was cast on a newly peeled mica surface and then dried. Data were analyzed using the XEN software (Park Systems, Korea).

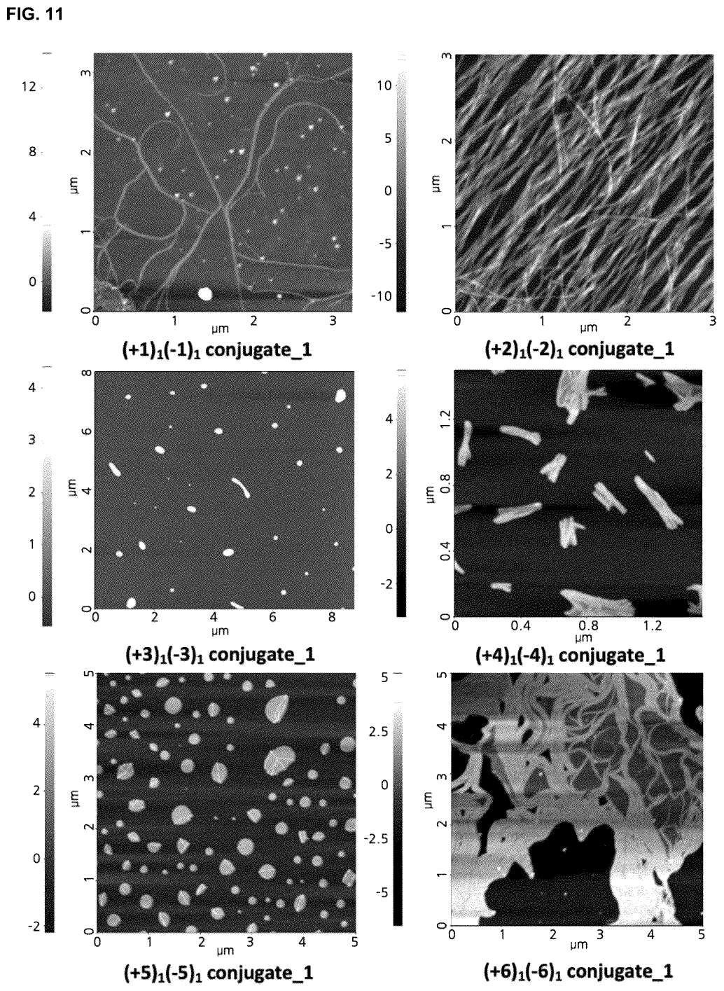

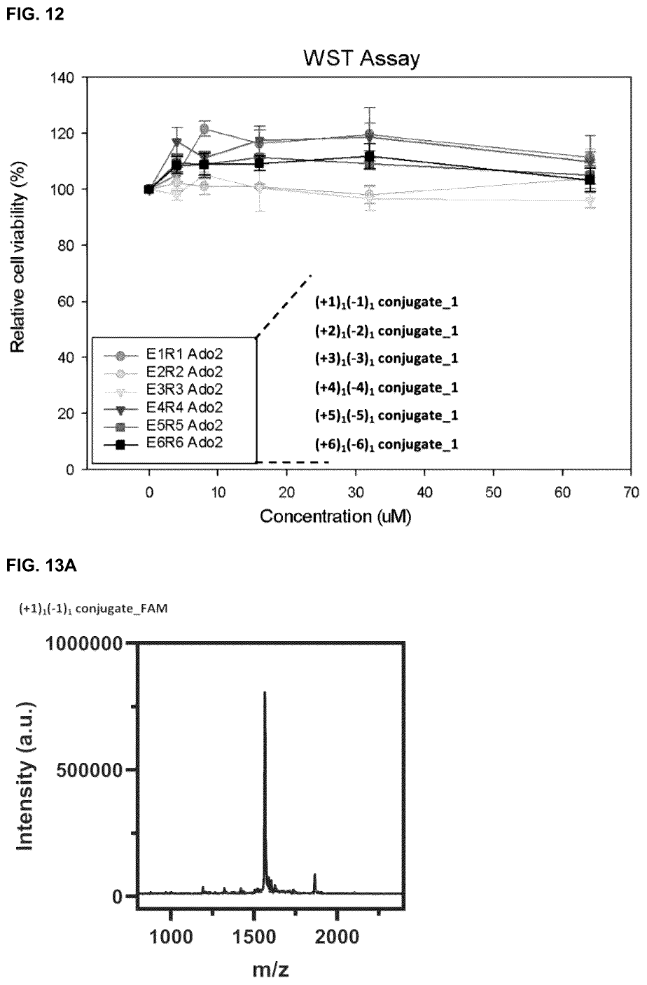

FIG. 11 shows the AFM images of Janus-type dendritic polyelectrolyte zwitterions of Examples 3-1 to 3-6 ((+1)₁(−1)₁-conjugate_1, (+2)₁(−2)₁-conjugate_1, (+3)₁(−3)₁-conjugate_1, (+4)₁(−4)₁-conjugate_1, (+5)₁(−5)₁-conjugate_1, and (+6)₁(−6)₁-conjugate_1) in DW solution.

The Janus-type dendritic polyelectrolyte zwitterions of Examples 3-1, 3-2, 3-4, and 3-6 formed supramolecular nanostructures in the form of elongated nanofibers, and the Janus-type dendritic polyelectrolyte zwitterions of Examples 3-3 and 3-5 formed round supramolecular nanostructures.

### 3) Cytotoxicity Analysis

The cytotoxicity of the Janus-type dendritic polyelectrolyte zwitterions (JDPZs) according to the present disclosure was analyzed. Since the JDPZ contains both a polyelectrolyte and a zwitterion in a single molecule, it is expected to have non-fouling property and low toxicity, while also having advantageous intracellular delivery efficiency due to its interaction with the cell membrane.

Cytotoxicity was analyzed using HeLa cells. Specifically, HeLa cells were subcultured in DMEM supplemented with 10% FBS (fetal bovine serum), dispensed into each well at 1×10⁴ cells/well, and cultured at 37° C. for 24 hours. The cells were treated with the samples (Examples 3-1 to 3-6) at various concentrations and then cultured at 37° C. for 4 hours. WST-8 solution was added to the cultured cells, and absorbance was measured at 450 nm with a microplate reader (Perkin-Elmer, USA) 4 hours later.

FIG. 12 shows a result of analyzing the cytotoxicity of (+M)₁(−M)₁ conjugate_1 of Examples 3-1 to 3-6 depending on concentration (0, 4, 8, 16, 32, 64 μM). It was confirmed that the Janus-type dendritic polyelectrolyte zwitterions (JDPZs) of Examples 3-1 to 3-6 ((+1)₁(−1)₁-conjugate_1, (+2) 1 (−2) 1-conjugate_1, (+3)₁(−3)₁-conjugate_1, (+4)₁(−4)₁-conjugate_1, (+5)₁(−5)₁-conjugate_1, and (+6)₁(−6)₁-conjugate_1) are stable substance that do not cause toxicity to the cells.

### Experimental Example 6. Characterization of Fluorescent Material-Introduced G1 Janus-Type Dendritic Polyelectrolyte Zwitterion (JDPZ_FAM)

### 1) MALDI-TOF Mass Spectrometry

The synthesis of the fluorescent material-introduced G1 Janus-type dendritic polyelectrolyte zwitterions prepared in Examples 4-1 to 4-12 (JDPZ, (+M)₁(−M)₁ conjugate_FAM) was confirmed by MALDI-TOF analysis.

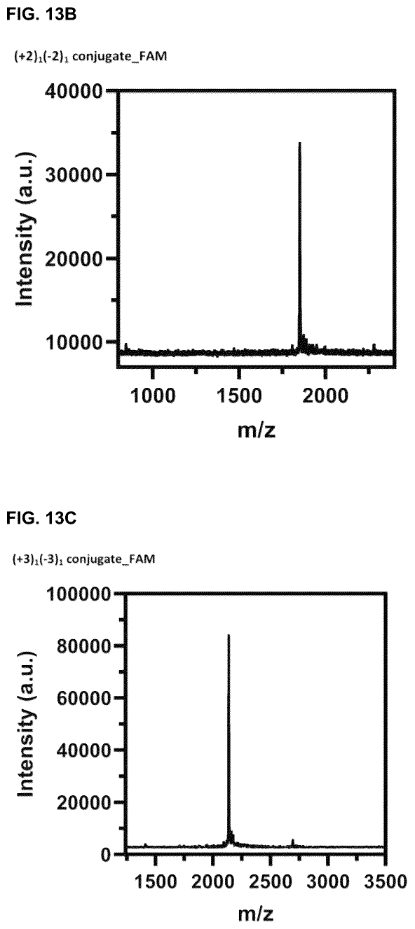

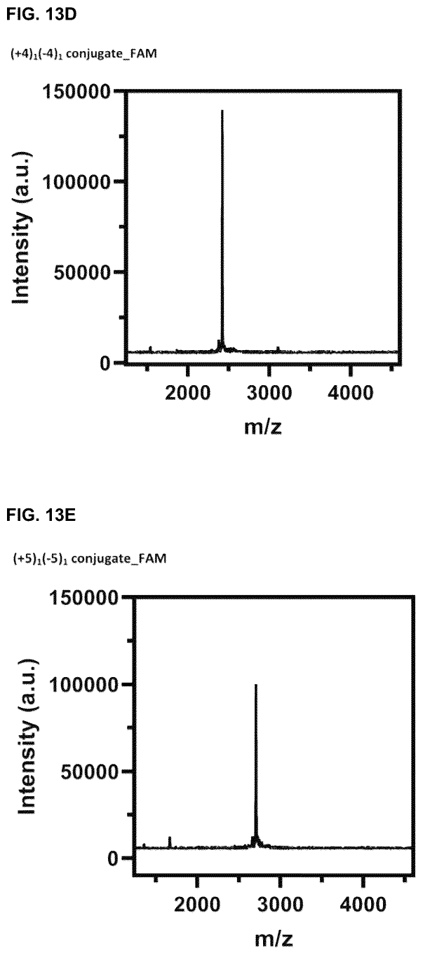

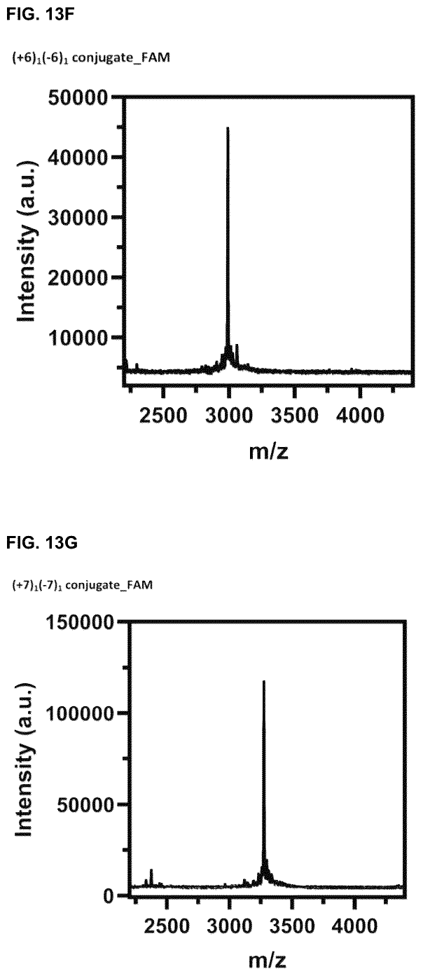

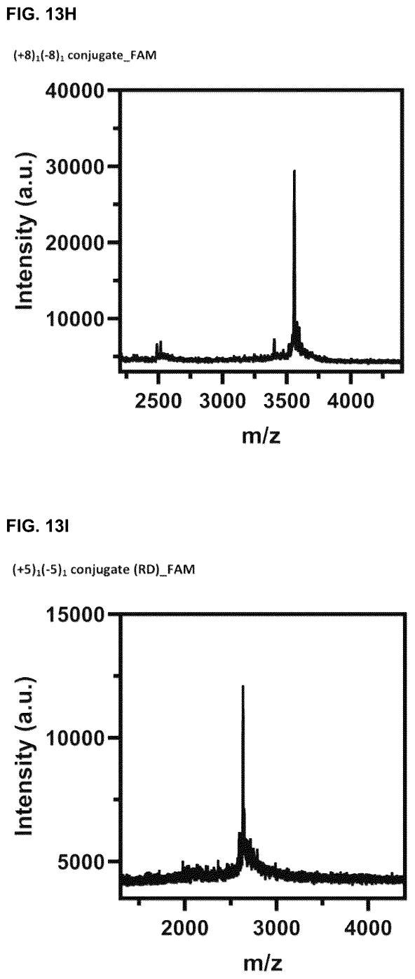

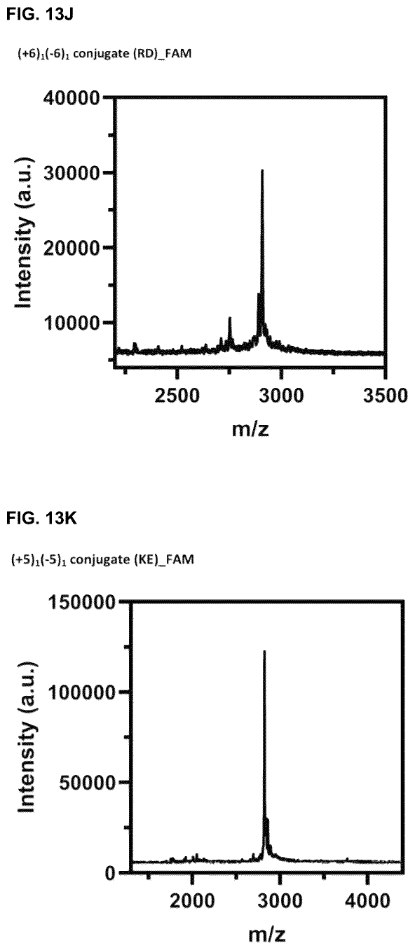

FIGS. 13a to 131 show the MALDI-TOF spectra of G1 Janus-type dendritic polyelectrolyte zwitterions (JDPZs, (+M)₁(−M)₁ conjugate_FAM) prepared in Examples 4-1 to 4-12 into which a fluorescent material is introduced. FIG. 13a: (+1)₁(−1)₁ conjugate_FAM of Example 4-1, FIG. 13b: (+2)₁(−2)₁ conjugate_FAM of Example 4-2, FIG. 13c: (+3)₁(−3)₁ conjugate_FAM of Example 4-3, FIG. 13d: (+4)₁(−4)₁ conjugate_FAM of Example 4-4, FIG. 13e: (+5)₁(−5)₁ conjugate_FAM of Example 4-5, FIG. 13f: (+6)₁(−6)₁ conjugate_FAM of Example 4-6, FIG. 13g: (+7)₁(−7)₁ conjugate_FAM of Example 4-7, FIG. 13h: (+8)₁(−8)₁ conjugate_FAM of Example 4-8, FIG. 13i: (+5)₁(−5)₁ conjugate (RD)_FAM of Example 4-9, FIG. 13j: (+6)₁(−6)₁ conjugate (RD) FAM of Example 4-10, FIG. 13k: (+5)₁(−5)₁ conjugate (KE)_FAM of Example 4-11, FIG. 13l: (+6)₁(−6)₁ conjugate (KE)_FAM of Example 4-12.

As shown in FIGS. 13a to 131, it was confirmed that the G1 Janus-type dendritic polyelectrolyte zwitterions into which the fluorescent material was introduced, prepared in Examples 4-1 to 4-12, were also synthesized successfully.

### 2) Intracellular Delivery Efficiency

The intracellular delivery efficiency of the G1 Janus-type dendritic polyelectrolyte zwitterions into which the fluorescent material was introduced (JDPZ, (+M)₁(−M)₁ conjugate_FAM), prepared in Examples 4-1 to 4-12, was investigated. The intracellular delivery efficiency was analyzed using HeLa cells. First, HeLa cells were subcultured in DMEM supplemented with 10% FBS (fetal bovine serum), dispensed into each well at 1-3×10⁴ cells/well, and cultured at 37° C. for 24 hours. The cells were treated with JDPZ ((+M)₁(−M)₁ conjugate_FAM) (Examples 4-1 to 4-12) at a concentration of 4 μM, 8 μM or 32 μM, respectively, and incubated at 37° C. for 4 hours. Afterwards, the cells were washed with Dulbecco's phosphate-buffered saline (DPBS), trypsinized, resuspended in DPBS supplemented with 10% cell dissociation buffer (GIBCO) and 1% FBS, and then analyzed using a flow cytometer (BD LSR II SORT flow cytometer, BD Bioscience, USA) for analysis of intracellular fluorescence.

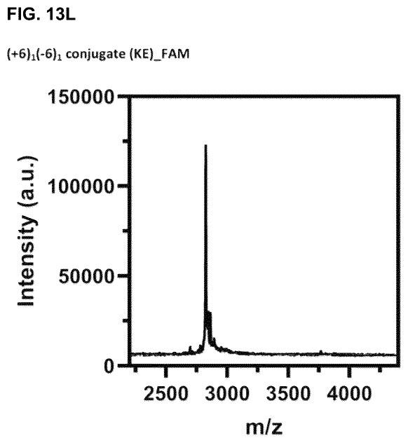

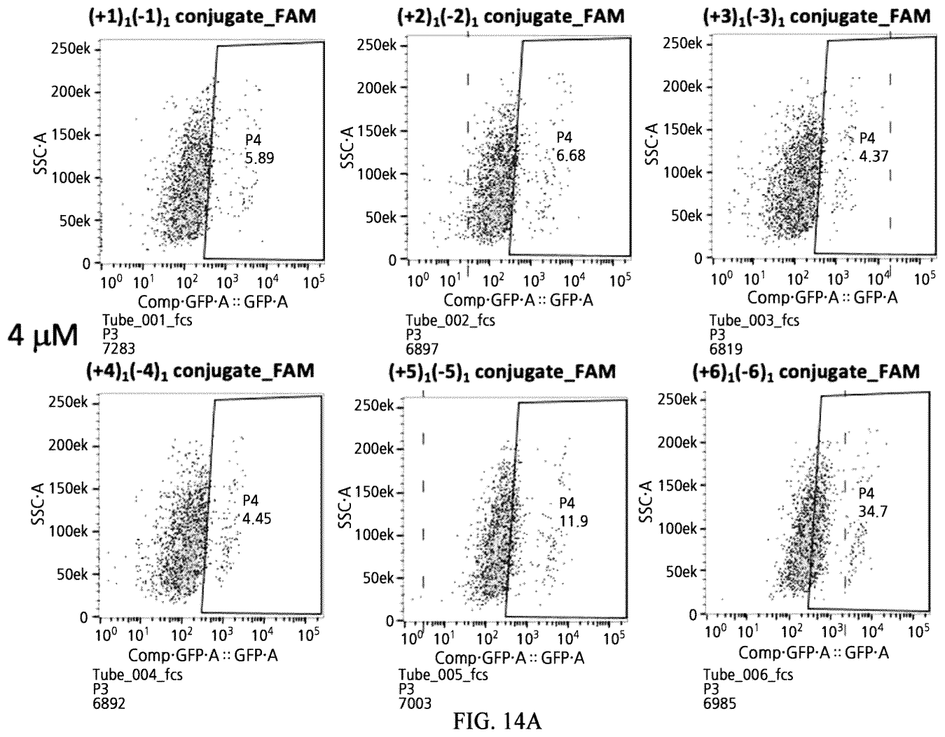

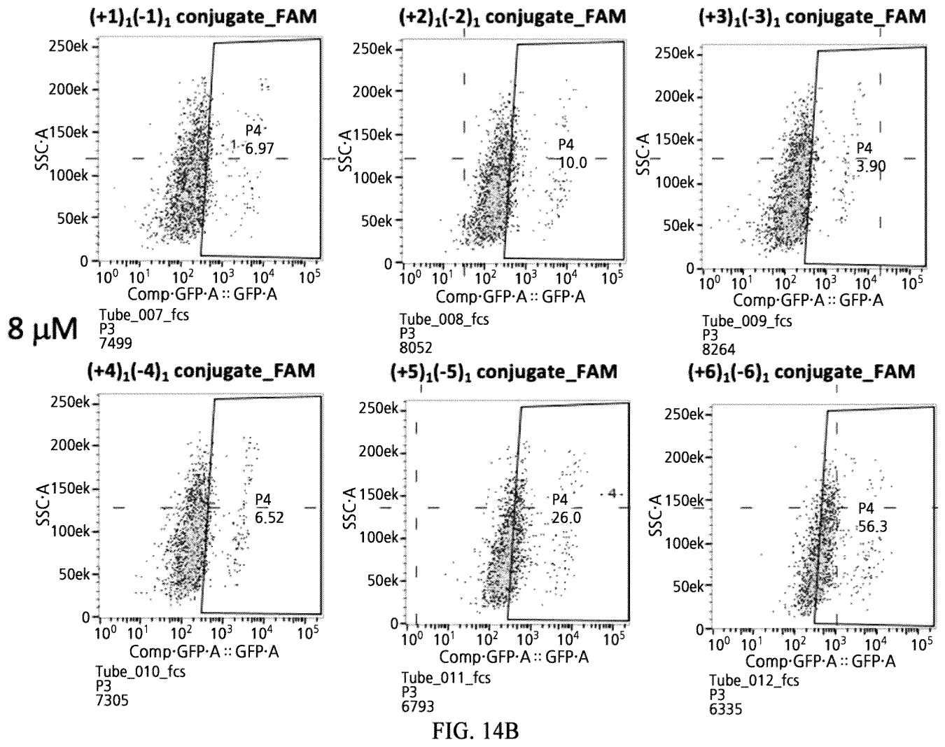

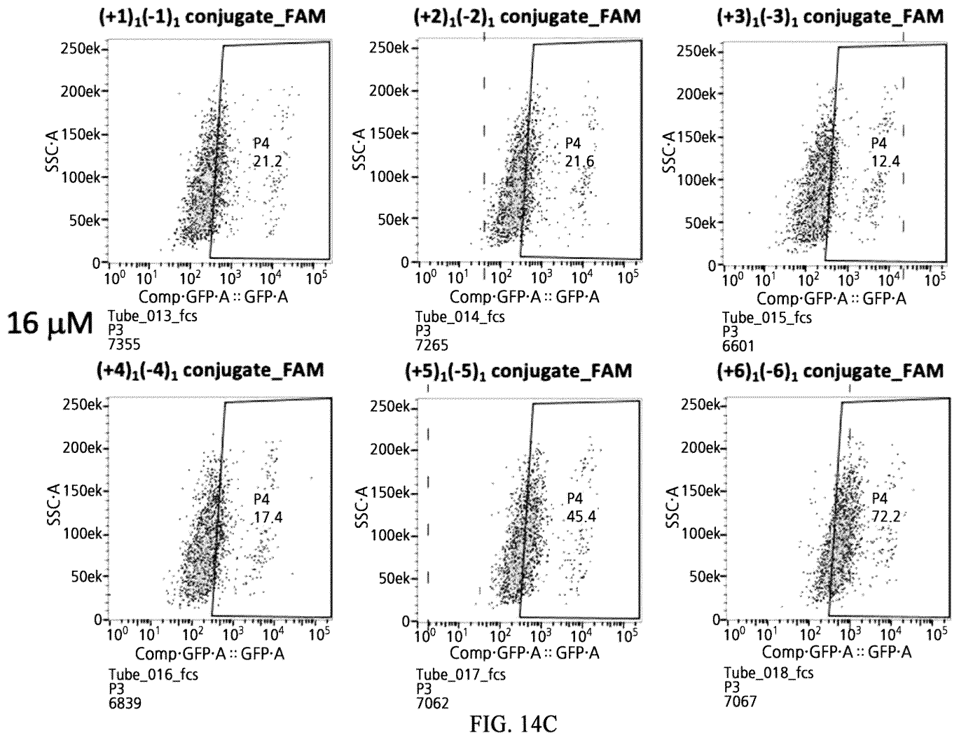

FIGS. 14a to 14c show the result of analyzing the intracellular delivery efficiency (degree of internalization) of JDPZ ((+M)₁(−M)₁ conjugate_FAM) prepared in Examples 4-1 to 4-6 by flow cytometry (FACS). FIG. 14a shows the result for 4 M JDPZ ((+M)₁(−M)₁ conjugate_FAM) prepared in Examples 4-1 to 4-6, FIG. 14b shows the result for 8 M JDPZ ((+M)₁(−M)₁ conjugate_FAM) prepared in Examples 4-1 to 4-6, and FIG. 14c shows the result for 16 M JDPZ ((+M)1(−M)1 conjugate_FAM) prepared in Examples 4-1 to 4-6.

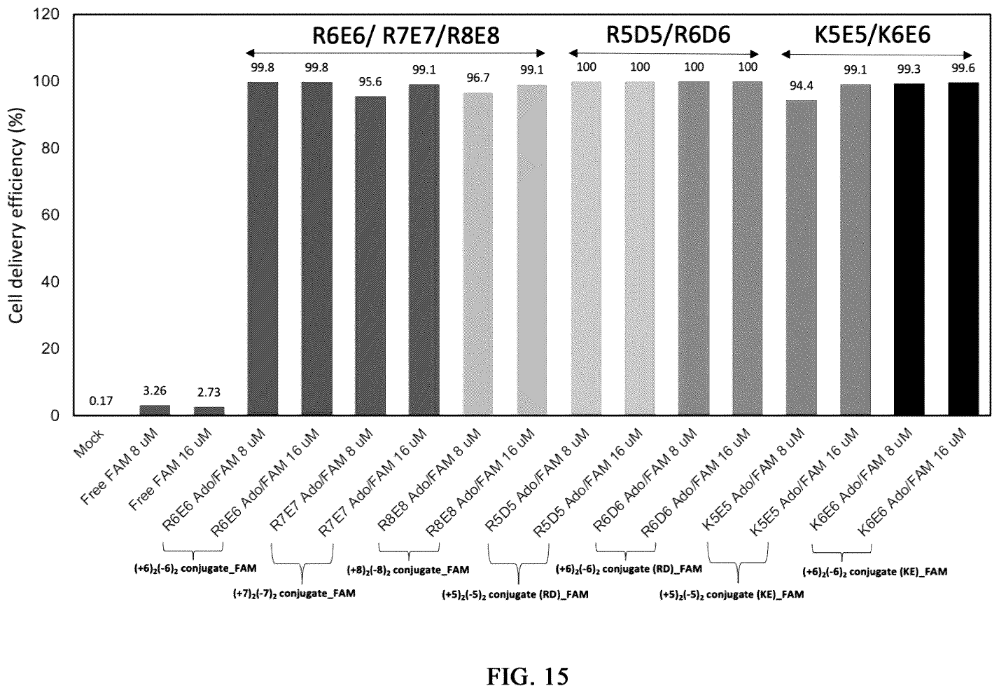

FIG. 15 shows the result of analyzing the intracellular delivery efficiency of the JDPZs prepared in Examples 4-6 to 4-12 ((+M)₁(−M)₁ conjugate_FAM) by flow cytometry (FACS).

As shown in FIGS. 14a to 14c, all of the G1 Janus-type dendritic polyelectrolyte zwitterions of Examples 4-1 to 4-6 into which the fluorescent material was introduced were delivered efficiently into cells, and the intracellular delivery efficiency was proportional to the concentration (dose-response relationship).

As shown in FIG. 15, the G1 Janus-type dendritic polyelectrolyte zwitterions of Examples 4-6 to 4-12 into which the fluorescent material was introduced showed a high internalization efficiency of 94.4% to 100%. The internalization efficiency increased in proportion to the concentration.

### Experimental Example 7. Characterization of G2 Janus-Type Dendritic Polyelectrolyte Zwitterion

### 1) MALDI-TOF Mass Spectrometry

The synthesis of the G2 Janus-type dendritic polyelectrolyte zwitterions prepared in Examples 5-1 to 5-4 (JDPZ, (+M)₂(−M)₂ conjugate_1) and the G2 Janus-type dendritic polyelectrolyte zwitterions prepared in Comparative Examples 1-1 to 1-3 was confirmed by MALDI-TOF analysis.

FIGS. 16a to 16d show the MALDI-TOF spectra of G2 Janus-type dendritic polyelectrolyte zwitterions prepared in Examples 5-1 to 5-4 (JDPZ ((+M)₂(−M)₂ conjugate_1). FIG. 16a: (+3)₂(−3)₂ conjugate_1 prepared in Example 5-1, FIG. 16b: (+4)₂(−4)₂ conjugate_1 prepared in Example 5-2, FIG. 16c: (+5)₂(−5)₂ conjugate_1 prepared in Example 5-3, FIG. 16d: (+6)₂(−6)₂ conjugate_1 prepared in Example 5-4.

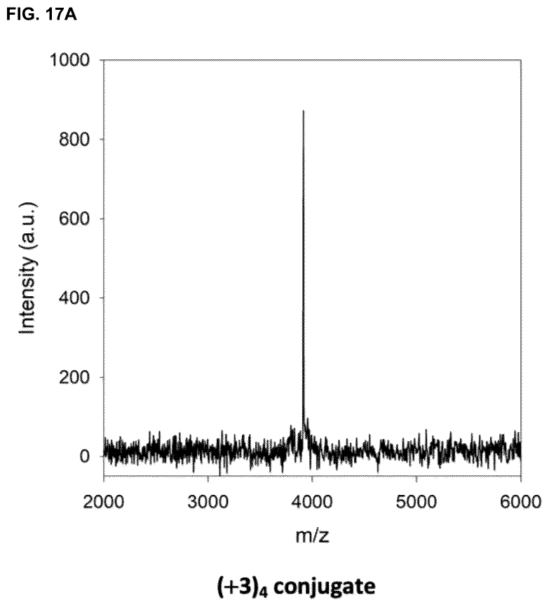

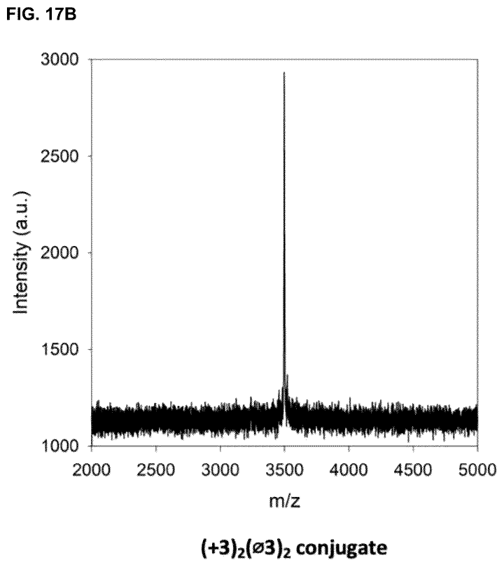

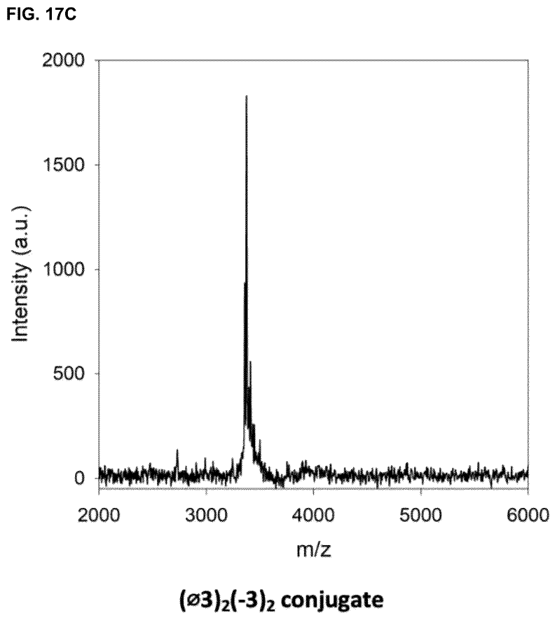

FIG. 17a shows the MALDI-TOF spectrum of (+3)₄ conjugate prepared in Comparative Example 1-1, FIG. 17b shows the MALDI-TOF spectrum of (+3)₂(Ø)₂ conjugate prepared in Comparative Example 1-2, and FIG. 17c shows the MALDI-TOF spectrum of (Ø)₂(−3)₂ conjugate prepared in Comparative Example 1-3.

As shown in FIGS. 16a to 16d and FIGS. 17a to 17c, it was confirmed that the G2 Janus-type dendritic polyelectrolyte zwitterions prepared in Examples 5-1 to 5-4 and the G2 Janus-type dendrimers prepared in Comparative Examples 1-1 to 1-3 were also synthesized successfully.

### 2) Structural Analysis

Each of the G2 Janus-type dendritic polyelectrolyte zwitterions of Examples 5-1 to 5-4 (JDPZ, (+M)₂(−M)₂ conjugate_1) was added to DW to prepare an aqueous solution and analyzed by AFM. AFM was performed using the NX10 system (Park Systems, Korea) in non-contact mode. 1 μL of the sample was cast on the newly peeled mica surface and then dried. Data were analyzed using the XEN software (Park Systems, Korea).

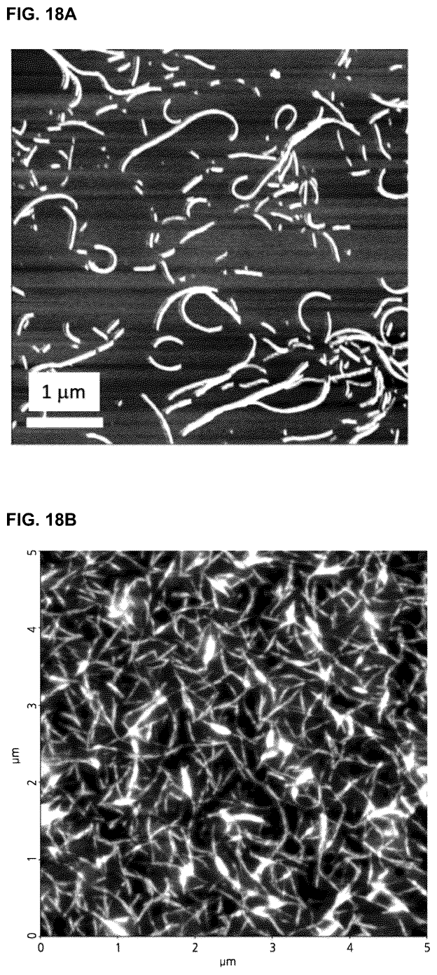

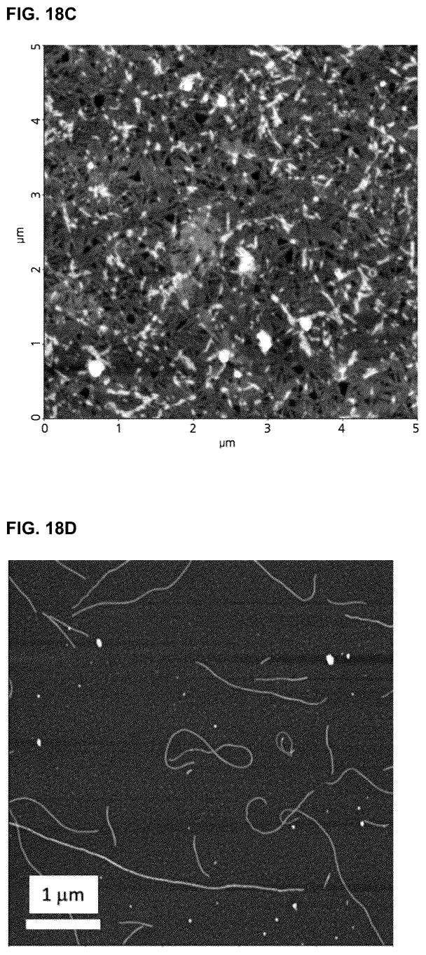

FIGS. 18a to 18d show the AFM images of G2 Janus-type dendritic polyelectrolyte zwitterions of Examples 5-1 to 5-4 (JDPZs, (+M)₂(−M)₂ conjugate_1). FIG. 18a: (+3)₂(−3)₂ conjugate_1 prepared in Example 5-1, FIG. 18b: (+4)₂(−4)₂ conjugate_1 prepared in Example 5-2. FIG. 18c: (+5)₂(−5)₂ conjugate_1 prepared in Example 5-3, FIG. 18d: (+6)₂(−6)₂ conjugate_1 prepared in Example 5-4.

It was confirmed that the G2 Janus-type dendritic polyelectrolyte zwitterions of Examples 5-1 to 5-4 (JDPZ, (+M)₂(−M)₂ conjugate_1) were supramolecular structures in the form of elongated nanofibers.

### 3) Cytotoxicity Analysis

The cytotoxicity of the G2 Janus-type dendritic polyelectrolyte zwitterions according to the present disclosure (JDPZ, (+M)₂(−M)₂ conjugate_1) was analyzed using HCT116 cells (human colon cancer cells). Specifically, HCT116 cells were subcultured in DMEM supplemented with 10% FBS (fetal bovine serum), dispensed into each well at 1×10⁴ cells/well, and cultured at 37° C. for 24 hours. The cells were treated with the samples (Example 5-1 and Comparative Examples 1-1 to 1-3) at various concentrations (0, 1.25, 2.5, 5, 10 μM), respectively, and incubated at 37° C. for 4 hours. WST-8 solution was added to the cultured cells, and absorbance was measured at 450 nm with a microplate reader (Perkin-Elmer, USA) 4 hours later.

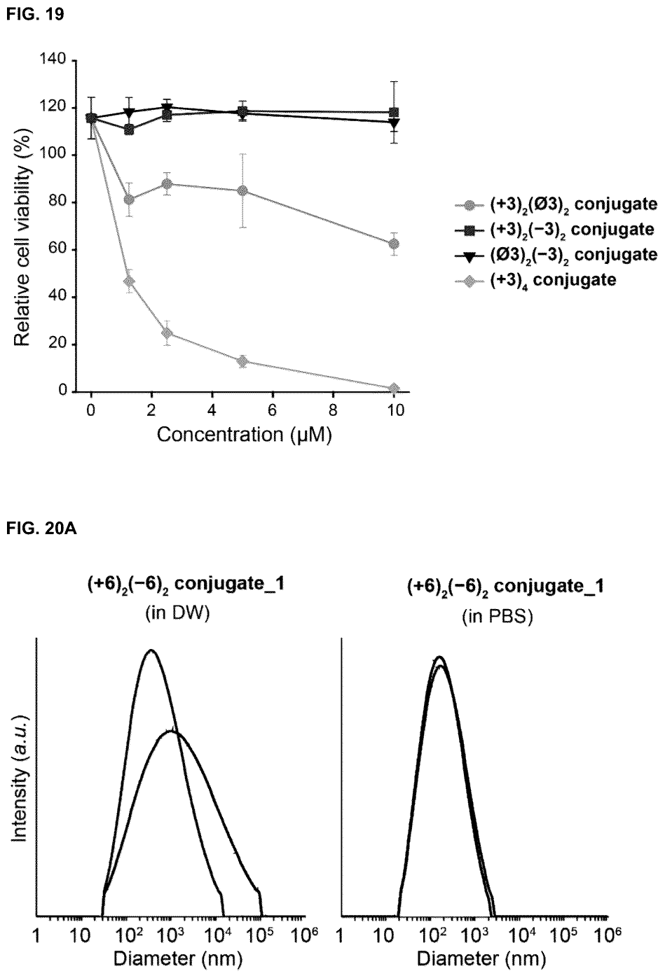

FIG. 19 shows a result of analyzing the cytotoxicity of a G2 Janus-type dendritic polyelectrolyte zwitterion prepared in Example 5-1 (JDPZ, (+M)₂(−M)₂ conjugate_1) and G2 Janus-type dendrimers prepared in Comparative Examples 1-1 to 1-3.

As shown in FIG. 19, Example 5-1 (JDPZ, (+3)₂(−3)₂ conjugate_1) was confirmed to be a safe material that does not exhibit cytotoxicity. On the other hand, Comparative Example 1-1 ((+3)₄ conjugate) or Comparative Example 1-2 ((+3)₂(Ø3)₂ conjugate) in which any one peptide was present in excess showed very high toxicity to the cells. Comparative Example 1-2 still exhibited cytotoxicity although the cytotoxicity was lower than that of Comparative Example 1-1.

### 4) DLS Analysis

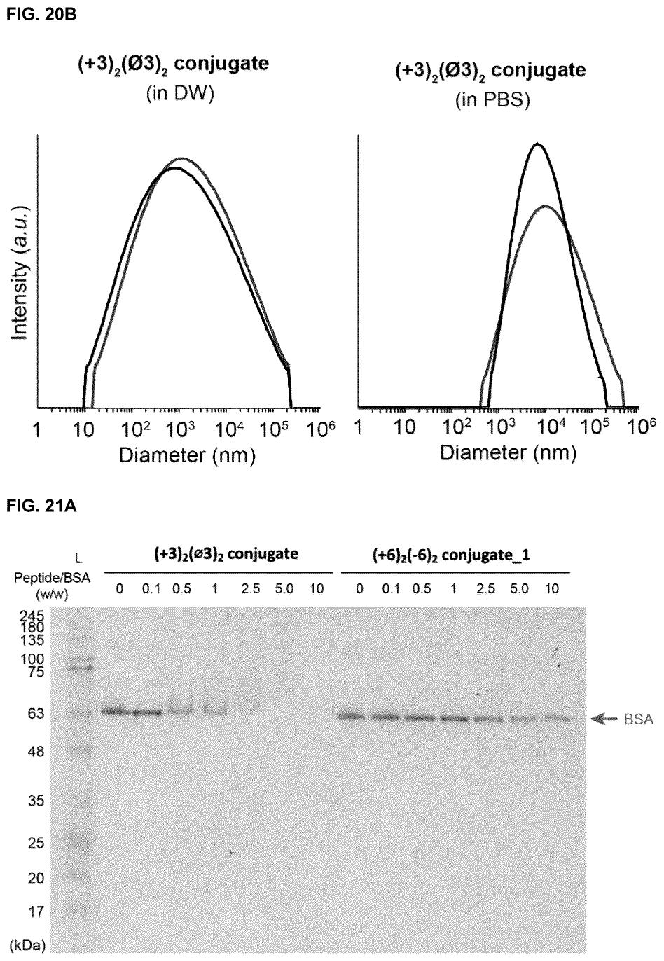

Each of the G2 Janus-type dendritic polyelectrolyte zwitterions prepared in Examples 5-1 and 5-4 and Comparative Example 1-2 (JDPZ, (+M)₂(−M)₂ conjugate_1) was mixed DW and PBS to prepare a solution and analyzed by DLS. Size and size distribution were analyzed by dynamic light scattering (DLS) using an ELS-Z1000 particle size analyzer (Otsuka, Japan), and the result is shown in Table 1 and FIGS. 20a and 20b.

FIGS. 20a and 20b show the DLS spectra of a Janus-type dendritic polyelectrolyte zwitterion ((+6)₂(−6)₂ conjugate_1) of Example 5-4 (a) and a Janus-type dendrimer ((+3)₂(Ø3)₂ conjugate) of Comparative Example 1-2 (b) under different solution conditions (DW or PBS). The DLS result for the Janus-type dendritic polyelectrolyte zwitterions of Examples 5-1 and 5-4 ((+3)₂(−3)₂ conjugate_1 and (+6)₂(−6)₂ conjugate_1) and the Janus-type dendrimer of Comparative Example 1-2 ((+3)₂(Ø3)₂ conjugate) under different solution conditions (DW or PBS) is shown in Table 1.

TABLE 1

| | | Diameter | Diameter | Zeta |
|---|---|---|---|---|
| | Net | (nm) in | (nm) in | potential |
| | charge | DW | PBS | (mV) |
| Example 5-1 | 0 | 109.8 | 75.4 | +42.3 |
| Example 5-4 | 0 | 649.0 | 128.9 | +35.2 |
| Comparative | +6 | 742.3 | 5,688.2 | +52.8 |
| Example 1-2 | | | | |

As shown in Table 1 and FIGS. 20a and 20b, it was confirmed that the Janus-type dendritic polyelectrolyte zwitterion of Example 5-4 ((+6)₂(−6)₂ conjugate_1) has a significantly smaller size or diameter and a narrower size distribution under the PBS condition as compared to under the DW condition.

That is to say, in the Janus-type dendritic polyelectrolyte zwitterions of Examples 5-1 and 5-4 according to the present disclosure, the packing parameters of the amphiphilic molecule decrease as the volume fraction of the water-soluble electrolyte-zwitterion-based dendrimer portion increases at high salt concentrations such as in PBS, due to the antipolyelectrolyte effect, which causes the size of the self-assembled supramolecular structure to decrease under the PBS condition as compared to under the DW condition.

On the other hand, the positively charged Janus-type dendrimer prepared in Comparative Example 1-2 ((+3)₂(Ø3)₂ conjugate) had a larger size in PBS than in DW. In other words, it was confirmed that the size or diameter of the positively charged dendrimer of Comparative Example 1-2 increases significantly under the PBS condition. This is due to the polyelectrolyte effect, which is a general characteristic of polyelectrolytes.

From these DLS results, it can be seen that the Janus-type dendritic polyelectrolyte zwitterion and the Janus-type dendrimer consisting only of positive charges have completely different solution behaviors. Therefore, the Janus-type dendritic polyelectrolyte zwitterions of Examples 5-1 and 5-4 can maintain a more stable structure than the dendrimers of Comparative Examples 1-1 to 1-2 under in-vivo conditions, making them very suitable for in-vivo applications.

### 5) Characterization of Non-Fouling Property for BSA

It was confirmed through EMSA (electrophoretic mobility shift assay) that the dendrimer structure of the present disclosure has non-fouling property for various proteins present in the body when administered in vivo.

First, 1 μg of BSA, an adhesive protein present in the blood at a high proportion, was prepared as a model protein and mixed with the dendritic polyelectrolyte zwitterion of Example 5-4 (JDPZ, (+6)₂(−6)₂ conjugate_1) at various concentration ratios. The mixing ratio was peptide/BSA ratio, wherein the peptide/BSA ratio was peptide/BSA (w/w). After reacting the mixture at room temperature for 1 hour, electrophoresis was performed on a 12% native polyacrylamide gel electrophoresis (PAGE) gel at 20 mA for 90 minutes. For visualization, the bands were stained with Coomassie brilliant blue R-250.

The Janus-type dendrimer structure of Comparative Example 1-2 ((+3)₂(Ø3)₂ conjugate) was used as a control group instead of the JDPZ of Example 5-4 ((+6)₂(−6)₂ conjugate_1).

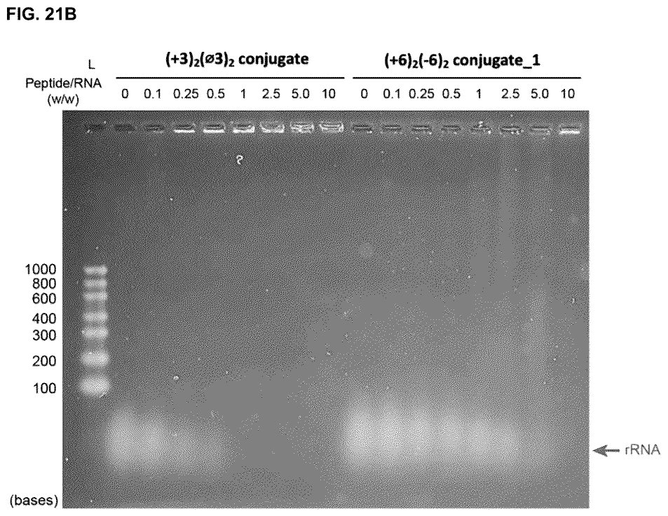

FIG. 21a shows an EMSA result of analyzing the non-fouling property of a Janus-type dendritic polyelectrolyte zwitterion (JDPZ ((+6)₂(−6)₂ conjugate_1)) structure of Example 5-4 and a Janus-type dendrimer ((+3)₂(Ø3)₂ conjugate) of Comparative Example 1-2 for proteins. Here, ‘L’ is a protein marker.

As shown in FIG. 21a, it was confirmed that the Janus-type dendrimer of Comparative Example 1-2 ((+3)₂(Ø3)₂ conjugate) binds easily to BSA. That is to say, the Janus-type dendrimer of Comparative Example 1-2 ((+3)₂(Ø3)₂-(C12)₄) induces the EMS (electrophoretic mobility shift) of the BSA band by interacting with BSA, suggesting that it lacks non-fouling property.

On the other hand, it was confirmed that the Janus-type dendritic polyelectrolyte zwitterion of Example 5-4 (JDPZ ((+6)₂(−6)₂ conjugate_1)) hardly binds to BSA even under the high-concentration BSA condition. That is to say, it was confirmed that the Janus-type dendritic polyelectrolyte zwitterion of Example 5-4 (JDPZ ((+6)₂(−6)₂ conjugate_1)) stably maintains its structure and role without being disturbed by BSA even at high concentration and exhibits high non-fouling property.

### 6) Characterization of Non-Fouling Property for rRNA

It was confirmed through EMSA (electrophoretic mobility shift assay) that the dendrimer structure of the present disclosure has non-fouling property for various genetic materials present in the body when administered in vivo. 200 ng of yeast rRNA (ribosomal RNA) was prepared as a model genetic material and mixed with the Janus-type dendritic polyelectrolyte zwitterion of Example 5-4 (JDPZ ((+6)₂(−6)₂ conjugate_1)) at various concentration ratios. The mixing ratio was peptide/RNA ratio, wherein the peptide/RNA ratio was peptide/RNA (w/w). After reacting the mixture at room temperature for 1 hour, electrophoresis was performed on a 2% agarose gel at 90 V for 100 minutes. For visualization, the bands were stained with SYBR Gold nucleic acid gel stain (Invitrogen, USA).

The Janus-type dendrimer structure of Comparative Example 1-2 ((+3)₂(Ø3)₂ conjugate) was used as a control group instead of the JDPZ of Example 5-4 ((+6)₂(−6)₂ conjugate_1).

FIG. 21b shows an EMSA result of analyzing the non-fouling property of a Janus-type dendritic polyelectrolyte zwitterion (JDPZ ((+6)₂(−6)₂ conjugate_1)) of Example 5-4 and a Janus-type dendrimer ((+3)₂(Ø3)₂ conjugate) of Comparative Example 1-2 for genetic materials. ‘L’ is an RNA marker.

Referring to FIG. 21b, it was confirmed that the Janus-type dendrimer of Comparative Example 1-2 ((+3)₂(Ø3)₂ conjugate) forms a non-specific interaction with rRNA even under the low-concentration rRNA condition. This is consistent with the fact that, among the cell-penetrating peptides known to deliver genetic materials, arginine-rich CPP is also known to have the problem of strong non-specific interaction with genetic materials existing in vivo or in cells.

On the other hand, since the Janus-type dendritic polyelectrolyte zwitterion of Example 5-4 (JDPZ ((+6)₂(−6)₂ conjugate_1)) according to the present disclosure forms interaction only under high-concentration conditions in which rRNA is present in an amount exceeding 10 times, the Janus-type dendritic polyelectrolyte zwitterion of Example 5-4 (JDPZ ((+6)₂(−6)₂ conjugate_1)) has high non-fouling property for genetic materials such as nucleic acids.

### Experimental Example 8. Characterization of Fluorescent Material-Introduced G2 Janus-type dendritic polyelectrolyte zwitterion (JDPZ ((+M)₂(−M)₂ conjugate_FAM)

### 1) MALDI-TOF Mass Spectrometry

The synthesis of the fluorescent material-introduced G2 Janus-type dendritic polyelectrolyte zwitterions prepared in Examples 6-1 and 6-2 (JDPZ ((+M)₂(−M)₂ conjugate_FAM) was confirmed by MALDI-TOF analysis.

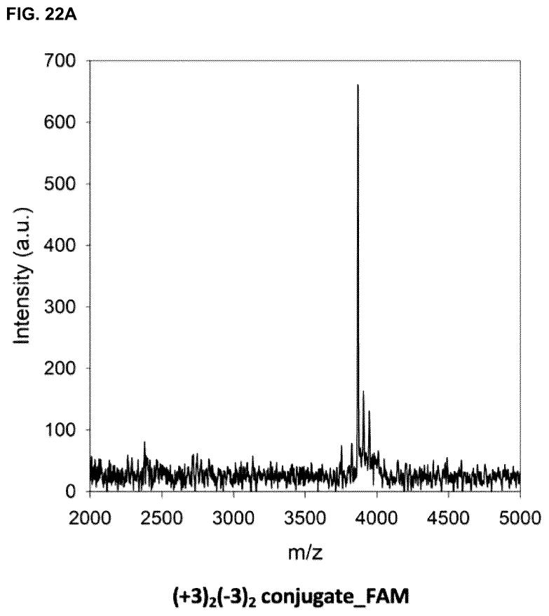

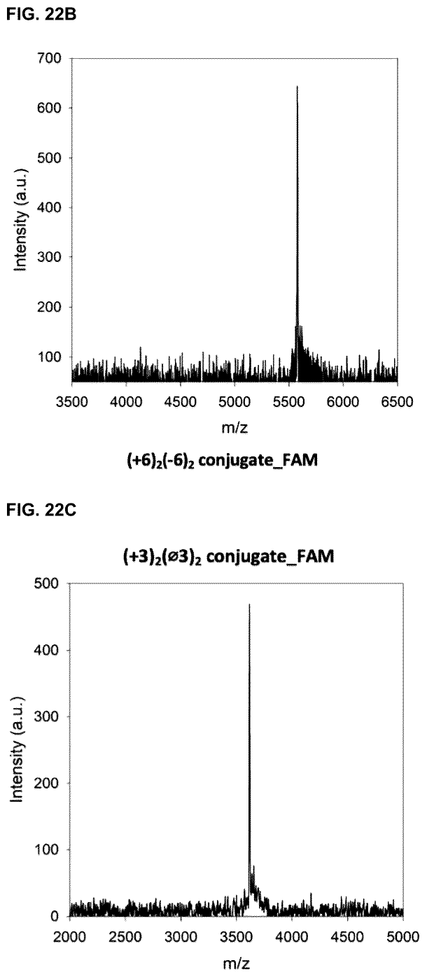

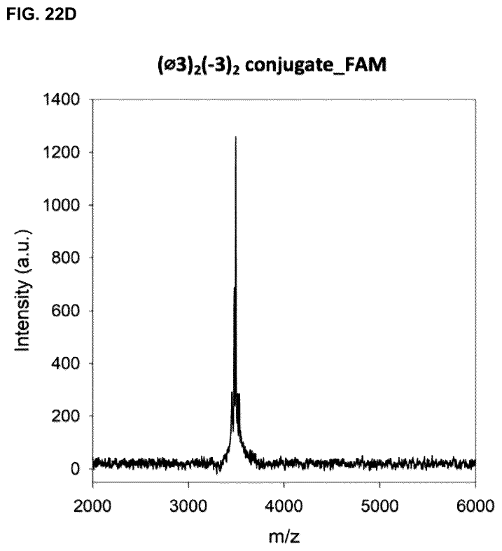

FIGS. 22a to 22d show the MALDI-TOF spectra of G2 Janus-type dendritic polyelectrolyte zwitterions (JDPZ ((+M)₂(−M)₂ conjugate_FAM) prepared in Examples 6-1 and 6-2 and Comparative Examples 2-1 and 2-2, into which a fluorescence material is introduced. FIG. 22a: (+3)₂(−3)₂ conjugate_FAM prepared in Example 6-1, FIG. 22b: (+6)₂(−6)₂ conjugate_FAM prepared in Example 6-2, FIG. 22c: (+3)₂(Ø3)₂ conjugate_FAM of Comparative Example 2-1, FIG. 22d: (Ø3)₂(−3)₂ conjugate_FAM of Comparative Example 2-2.

As shown in FIGS. 22a to 22d, it was confirmed that the fluorescent material-introduced G2 Janus-type dendritic polyelectrolyte zwitterions prepared in Examples 6-1 and 6-2 and Comparative Examples 2-1 and 2-2 were also synthesized successfully.

### 2) Intracellular Delivery Efficiency

To confirm the intracellular delivery efficiency, HCT116 cells were subcultured in DMEM supplemented with 10% FBS (fetal bovine serum), dispensed into each well at 1-3×10⁴ cells/well, and cultured at 37° C. for 24 hours. The cells were treated with the (+6)₂(−6)₂ conjugate_FAM prepared in Example 6-2 at a concentration of 10 μM and incubated at 37° C. for 4 hours. Afterwards, the cells were washed with Dulbecco's phosphate-buffered saline (DPBS), trypsinized, resuspended in DPBS supplemented with 10% cell dissociation buffer (GIBCO) and 1% FBS, and then analyzed using a flow cytometer (BD LSR II SORT flow cytometer, BD Bioscience, USA) for analysis of intracellular fluorescence.

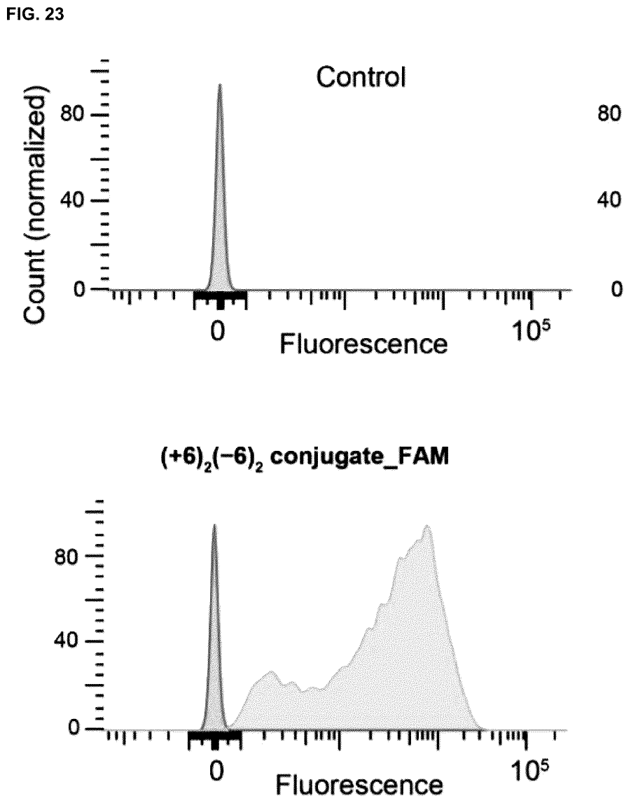

FIG. 23 shows a result of analyzing the intracellular delivery efficiency of JDPZ ((+6)₂(−6)₂ conjugate_FAM) prepared in Example 6-2 in HCT116 cells by flow cytometry. As shown in FIG. 23, it was confirmed that the fluorescent material-induced G2 Janus-type dendritic polyelectrolyte zwitterion of Example 6-2 has a high internalization efficiency of about 100%.

The dendritic polyelectrolyte zwitterion (DPZ) and the Janus-type dendritic polyelectrolyte zwitterion (JDPZ) according to the present disclosure were confirmed to be non-toxic and have high intracellular delivery efficiency. In contrast, the Janus-type dendrimers in which positively charged peptides form a strong supramolecular nanostructure (Comparative Examples 1-1, 1-2 and 2-1) have high cellular delivery efficiency but exhibit very high toxicity.

### 3) Intracellular Drug Delivery Efficiency

Since it was confirmed that the Janus-type dendritic polyelectrolyte zwitterion (JDPZ) is stably internalized in the cells, it was investigated whether the delivered drug is delivered into the cells by confocal laser scanning microscopy (CLSM).

HeLa cells were treated with the fluorescent material-introduced G2 Janus-type dendritic polyelectrolyte zwitterion prepared in Example 6-2 (JDPZ ((+6)₂(−6)₂ conjugate_FAM) and its intracellular location was measured by confocal laser scanning microscopy (CLSM). Specifically, HCT116 cells were inoculated in an 8-well Lab Tek II chamber cover glass system (Nunc, USA) at 2×10⁴ HeLa cells/well and cultured in DMEM containing 10% FBS (fetal bovine serum) and 1% Pen-Strep at 37° C. for 24 hours. Afterwards, the cells were washed with DPBS and treated with 10 M of the sample (Example 6-2) in Opti-MEM for 1 hour. Then, the cells were visualized using a confocal microscope (LSM 980, Carl Zeiss, Germany).

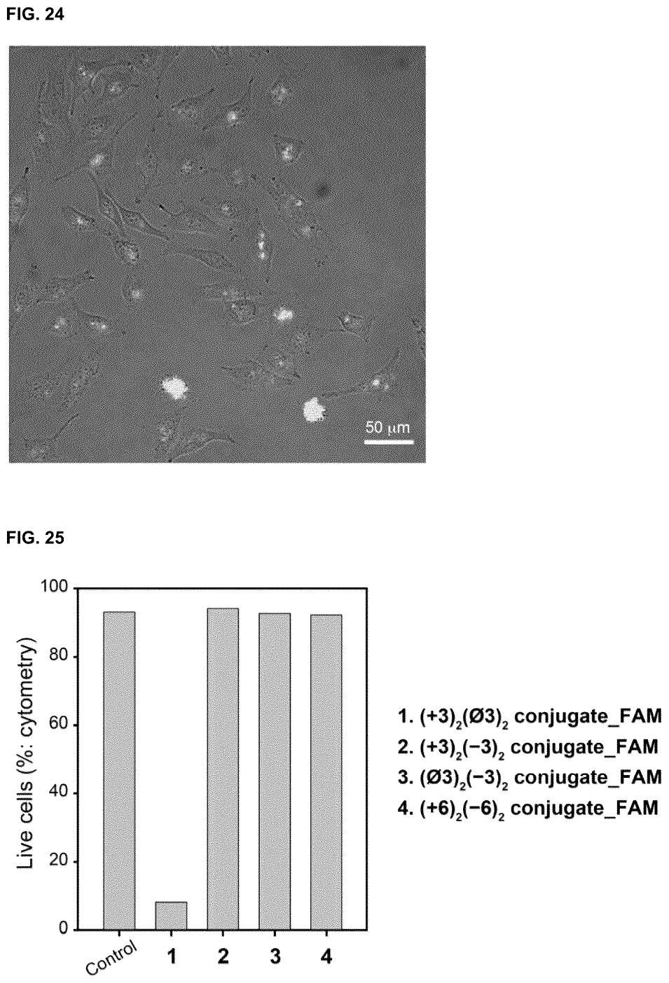

FIG. 24 shows a result of treating HeLa cells with a G2 Janus-type dendritic polyelectrolyte zwitterion (Example 6-2) into which a fluorescent label is introduced and analyzing the same by confocal microscopy (LSM 980, Carl Zeiss, Germany).

As shown in FIG. 24, it was confirmed that the fluorescent material-introduced G2 Janus-type dendritic polyelectrolyte zwitterion of Example 6-2 efficiently penetrated into the cells and successfully delivered the drug into the nucleus and cytoplasm of the cells.

### 4) Cytotoxicity

After treating HCT116 human colon cancer cells with each of Examples 6-1 and 6-2 and Comparative Examples 2-1 and 2-2, the proportion (%) of live cells was measured by flow cytometry (FACS) to measure cytotoxicity.

FIG. 25 shows a result of analyzing the cytotoxicity of G2 Janus-type dendritic polyelectrolyte zwitterions ((+M)₂(−M)₂ conjugate_FAM) into which a fluorescent material is introduced, prepared in Examples 6-1 and 6-2, and G2 Janus-type dendrimers into which the fluorescent material is introduced, prepared in Comparative Examples 2-1 and 2-2, by flow cytometry (FACS).

Example 6-1 ((+3)₂(−3)₂ conjugate_FAM) and Example 6-2 ((+6)₂(−6)₂ conjugate_FAM) did not show any cytotoxicity. In contrast, it was confirmed that Comparative Example 2-1 ((+3)₂(03)₂ conjugate_FAM) exhibits very high toxicity, killing 90% of the HCT116 cells.

In other words, although the dendritic polyelectrolyte zwitterions according to the present disclosure (Examples 1 to 6) contain a large number of positively charged peptides, since they have the special structure called the dendritic polyelectrolyte zwitterion, they were confirmed to be in a very stable state with no cytotoxicity in fluid.

### Experimental Example 9. Characterization of G2 Janus-type dendritic polyelectrolyte zwitterions ((+6)₂(−6)₂ conjugate_2 and (+6)₂(−6)₂ conjugate_3)

### 1) MALDI-TOF Mass Spectrometry

The G2 Janus-type dendritic polyelectrolyte zwitterions prepared in Example 7 and Example 8 ((+6)₂(−6)₂ conjugate_2 and (+6)₂(−6)₂ conjugate 3) were purified and analyzed by MALDI-TOF.

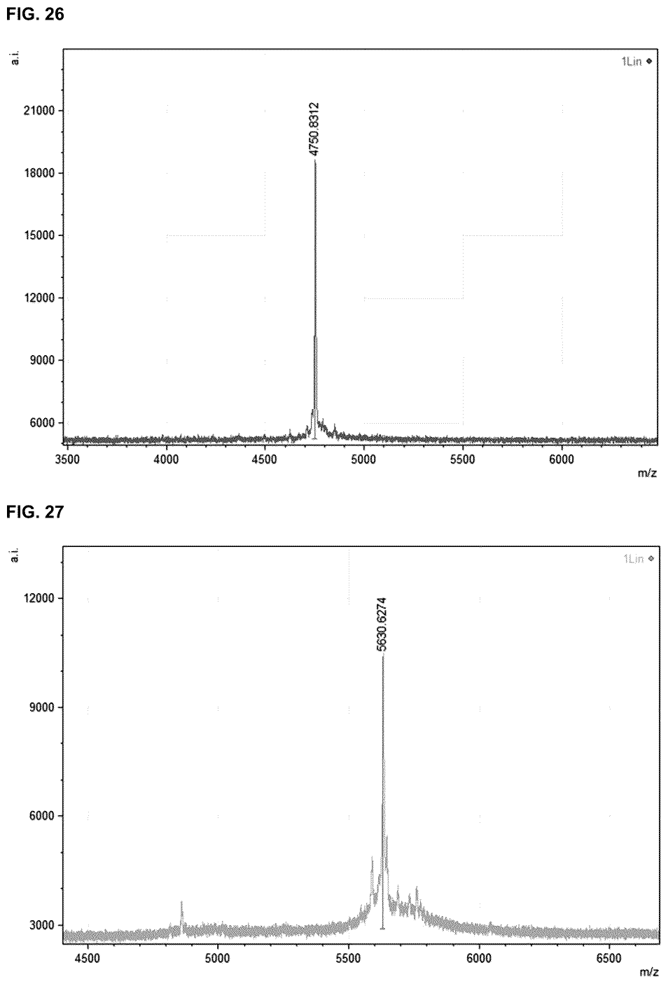

FIG. 26 shows the MALDI-TOF spectrum of a G2 Janus-type dendritic polyelectrolyte zwitterion prepared in Example 7 ((+6)₂(−6)₂ conjugate 2), and FIG. 27 shows the MALDI-TOF spectrum of the G2 Janus-type dendritic polyelectrolyte zwitterion prepared in Example 8 ((+6)₂(−6)₂ conjugate_3).

It was confirmed that the G2 Janus-type dendritic polyelectrolyte zwitterions prepared in Examples 7 and 8 were also synthesized successfully, regardless of the number and type of the hydrophobic compounds. It was confirmed that the synthesis was successful even when the hydrophilic amino acid derivative was further included instead of the hydrophobic compound.

### 2) Preparation of Janus-Type Dendritic Polyelectrolyte Zwitterion Complex Loaded with EGFP mRNA and Analysis of Intracellular Delivery Efficiency

A Janus-type dendritic polyelectrolyte zwitterion complex loaded with GFP mRNA was prepared by mixing the G2 Janus-type dendritic polyelectrolyte zwitterion prepared in Example 7 ((+6)₂(−6)₂_conjugate 2) or the G2 Janus-type dendritic polyelectrolyte zwitterion prepared in Example 8 ((+6)₂(−6)₂_conjugate 3) with 1 g of EGFP mRNA (SEQ ID NO: 167).

HeLa cells were subcultured in DMEM supplemented with 10% FBS (fetal bovine serum), dispensed into each well at 1-3×10⁴ cells/well, and cultured at 37° C. for 24 hours. The cells were treated with the (+6)₂(−6)₂_conjugate 2_complex or the (+6)₂(−6)₂_conjugate 3_complex at a concentration of 16 M or 32 M, respectively, and incubated at 37° C. for 4 hours. Afterwards, the cells were washed with Dulbecco's phosphate-buffered saline (DPBS), trypsinized, and resuspended in DPBS supplemented with 10% cell dissociation buffer (GIBCO) and 1% FBS. Then, the expressed eGFP (enhanced green fluorescent protein) was analyzed using a flow cytometer (BD LSR II SORT flow cytometer, BD Bioscience, USA).

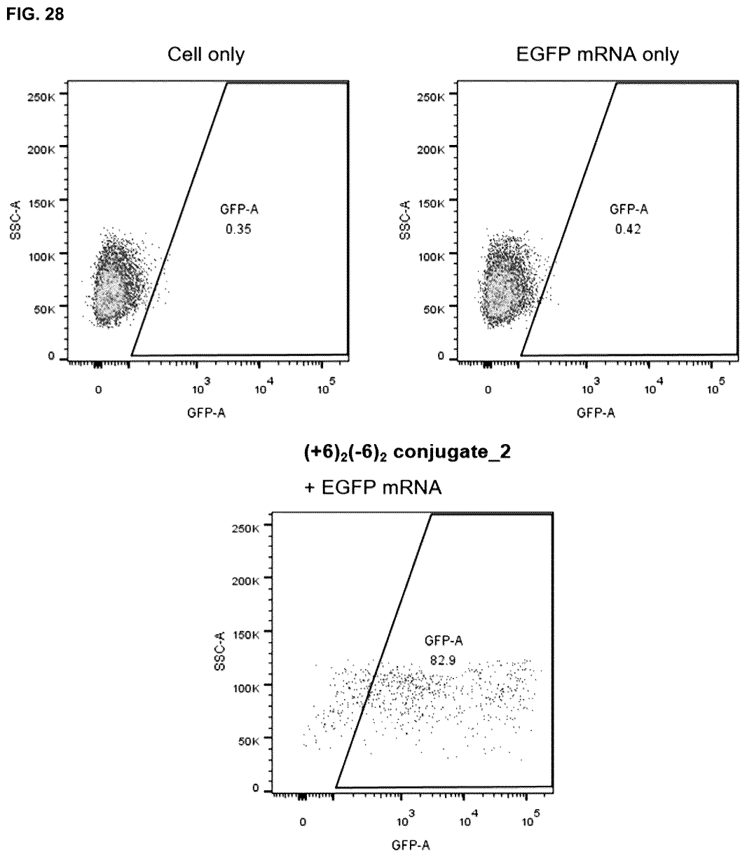

FIG. 28 shows a result of treating HeLa cells with the complex of the Janus-type dendritic polyelectrolyte zwitterion (+6)₂(−6)₂ conjugate_2 prepared in Example 7 and EGFP (enhanced green fluorescent protein) mRNA and quantifying the expressed GFP protein by flow cytometry.

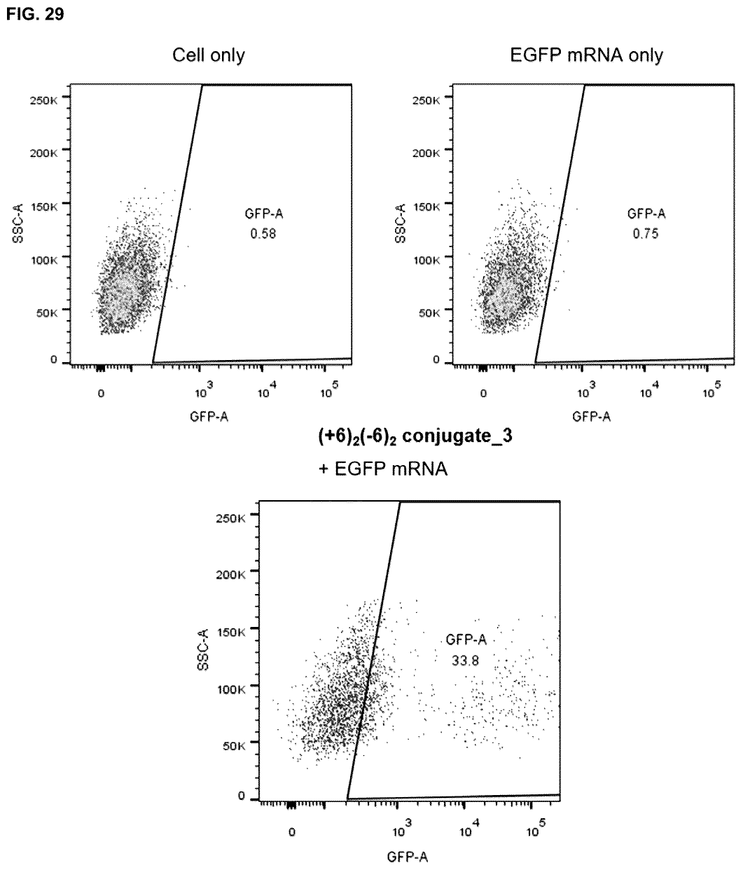

FIG. 29 shows a result of treating HeLa cells with the complex of the Janus-type dendritic polyelectrolyte zwitterion (+6)₂(−6)₂ conjugate 3 prepared in Example 8 and EGFP (enhanced green fluorescent protein) mRNA and quantifying the expressed GFP protein by flow cytometry.

As shown in FIGS. 28 and 29, it was confirmed that the Janus-type dendritic polyelectrolyte zwitterions of Examples 7 and 8 ((+6)₂(−6)₂ conjugate ⅔) not only effectively carried EGFP mRNA but also successfully delivered the mRNA into the nucleus and cytoplasm of the cells. The eGFP protein was expressed from the internalized EGFP mRNA with a high efficiency of 82.9%.

### 3) Preparation of Janus-Type Dendritic Polyelectrolyte Zwitterion Complex Loaded with EGFP mRNA and Analysis of In Vivo Delivery Efficiency

A Janus-type dendritic polyelectrolyte zwitterion complex loaded with GFP mRNA ((+6)₂(−6)₂_conjugate 2_complex) was prepared by mixing the G2 Janus-type dendritic polyelectrolyte zwitterion prepared in Example 7 ((+6)₂(−6)₂ conjugate 2) mixed with 5 ag of EGFP mRNA (SEQ ID NO: 167). The complex ((+6)₂(−6)₂_conjugate 2_complex) was subcutaneously injected into BALB/c nude mice at 0.1 mg/kg, and then the expressed eGFP (enhanced green fluorescenct protein) was imaged and quantified with IVIS (in vivo imaging system). As a control group, only EGFP mRNA was used instead of the complex.

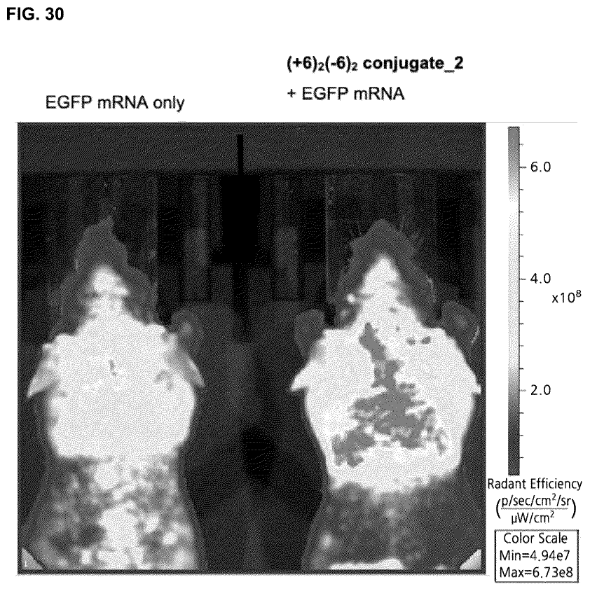

FIG. 30 shows the result of confirming the expression of the complex ((+6)₂(−6)₂_conjugate 2_complex) loaded with EGFP mRNA in vivo.

As shown in FIG. 30, the Janus-type dendritic polyelectrolyte zwitterion of Example 7 ((+6)₂(−6)₂ conjugate 2) not only effectively encapsulated EGFP mRNA but also successfully delivered the mRNA in vivo. The EGFP mRNA delivered in vivo was expressed as the eGFP protein. In contrast, when only EGFP mRNA was administered, no significant expression of the eGFP protein was observed.

In summary, in the present disclosure, a dendritic polyelectrolyte zwitterion was fabricated by attaching positively and negatively charged peptides to the ends of a polylysine dendrimer, and a Janus-shaped nanostructure was fabricated by binding a hydrophobic compound thereto. The Janus-type dendritic polyelectrolyte zwitterion does not exhibit toxicity although it contains a large amount of positively charged peptides, but exists stably by forming a supramolecular nanostructure in solution such as body fluid, and exhibits high non-fouling property for interaction with proteins and genetic materials existing in vivo. Therefore, when the Janus-type dendritic polyelectrolyte zwitterion according to the present disclosure is loaded with a genetic material, it can exhibit excellent effects of intracellular delivery and protein expression when administered to cells or in vivo. Accordingly, the Janus-type dendritic polyelectrolyte zwitterion of the present disclosure can be utilized as a carrier of a genetic material for safe in vivo delivery and can also be used as a therapeutic agent, a diagnostic agent, etc. using a genetic material.

1. A polyelectrolyte-zwitterion-based dendritic compound represented by the General Formula 1 or 2: 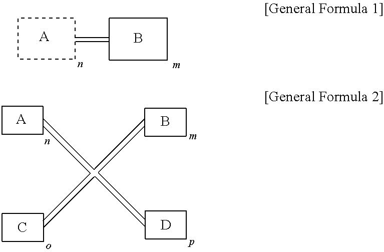
wherein
each of A and C is independently at least one selected from a group consisting of arginine (Arg, R), histidine (His, H), lysine (Lys, K), ornithine (Orn) and guanidine,
each of B and D is independently at least one selected from a group consisting of glutamic acid (Glu, E) and aspartic acid (Asp, D), and
each of n, m, o and p is independently an integer selected from 3 to 16.

2. The polyelectrolyte-zwitterion-based dendritic compound according to claim 1, wherein the dendritic compound is a polyelectrolyte-zwitterion-based dendritic compound wherein n, m, o and p are all the same under some conditions between pH 2 and pH 13.

3. The polyelectrolyte-zwitterion-based dendritic compound according to claim 1, wherein each of A and C is independently a positively charged peptide selected from a group consisting of SEQ ID NOS: 3 to 98.

4. The polyelectrolyte-zwitterion-based dendritic compound according to claim 1, wherein each of B and D is independently a negatively charged peptide selected from a group consisting of SEQ ID NOS: 99 to 162.

5. A dendritic polyelectrolyte zwitterion comprising:
a 1st to 5th generation dendrimer-based core; and
a positively charged peptide represented by SEQ ID NO: 1 and/or a negatively charged peptide represented by SEQ ID NO: 2 bound to the terminal functional group of the core:
[SEQ ID NO: 1]
[X₁]ₙ
[SEQ ID NO: 2]
[X₂]ₙ
wherein X₁ is any one selected from arginine (Arg, R), histidine (His, H), lysine (Lys, K), ornithine (Orn) and guanidine, and X₂ is glutamic acid (Glu, E) or aspartic acid (Asp, D), and
n is an integer from 3 to 16.

6. The dendritic polyelectrolyte zwitterion according to claim 5, wherein the dendrimer-based core is any one selected from a group consisting of a polyamidoamine (PAMAM) dendrimer, a polylysine dendrimer, a polyimine (PI) dendrimer, a polypropyleneimine (PPI) dendrimer, a polyester dendrimer, a polyamide dendrimer, a polyurethane dendrimer, a polyornithine dendrimer, a carbosilane dendrimer, a polyether dendrimer, a polyglutamic acid dendrimer, a polyaspartic acid dendrimer, a polyglycerol dendrimer and a polymelamine dendrimer.

7. The dendritic polyelectrolyte zwitterion according to claim 5, wherein the dendrimer-based core is of the 1st or 2nd generation.

8. The dendritic polyelectrolyte zwitterion according to claim 5, wherein the same number of the positively charged peptide represented by SEQ ID NO: 1 and the negatively charged peptide represented by SEQ ID NO: 2 are bound to the ends of the dendrimer-based core.

9. The dendritic polyelectrolyte zwitterion according to claim 5, wherein
if the dendrimer-based core is the 1st generation, one strand of the positively charged peptide chain represented by SEQ ID NO: 1 and one strand of the negatively charged peptide chain represented by SEQ ID NO: 2 are bound, and
if the dendrimer-based core is of the 2nd generation, two strands of the positively charged peptide chain represented by SEQ ID NO: 1 and two strands of the negatively charged peptide chain represented by SEQ ID NO: 2 are bound.

10. The dendritic polyelectrolyte zwitterion according to claim 5, wherein the positively charged peptide is represented by any one selected from a group consisting of SEQ ID NOS: 3 to 98.

11. The dendritic polyelectrolyte zwitterion according to claim 5, wherein the negatively charged peptide is represented by any one selected from a group consisting of SEQ ID NOS: 99 to 162.

12. A Janus-type dendritic polyelectrolyte zwitterion comprising:
a 1st to 5th generation dendrimer-based core;
a positively charged peptide represented by SEQ ID NO: 1 and/or a negatively charged peptide represented by SEQ ID NO: 2 bound to one terminal functional group based on the dendrimer core; and
a hydrophobic compound or an amino acid derivative bound to the other terminal functional group based on the dendrimer core:
[SEQ ID NO: 1]
[X₁]ₙ
[SEQ ID NO: 2]
[X₂]ₙ
wherein X₁ is any one selected from arginine (Arg, R), histidine (His, H), lysine (Lys, K), ornithine (Orn) and guanidine, and X₂ is glutamic acid (Glu, E) or aspartic acid (Asp, D), and
n is an integer from 3 to 16.

13. The Janus-type dendritic polyelectrolyte zwitterion according to claim 12, wherein the dendrimer-based core is any one selected from a group consisting of a polyamidoamine (PAMAM) dendrimer, a polylysine dendrimer, a polyimine (PI) dendrimer, a polypropyleneimine (PPI) dendrimer, a polyester dendrimer, a polyamide dendrimer, a polyurethane dendrimer, a polyornithine dendrimer, a carbosilane dendrimer, a polyether dendrimer, a polyglutamic acid dendrimer, a polyaspartic acid dendrimer, a polyglycerol dendrimer and a polymelamine dendrimer.

14. The Janus-type dendritic polyelectrolyte zwitterion according to claim 12, wherein the dendrimer-based core is of the 2nd or 3rd generation.

15. The Janus-type dendritic polyelectrolyte zwitterion according to claim 12, wherein the number of the positively charged peptide represented by SEQ ID NO: 1 and the number of the negatively charged peptide represented by SEQ ID NO: 2 bound to the terminals of the dendrimer-based core are the same.

16. The Janus-type dendritic polyelectrolyte zwitterion according to claim 12, wherein
the dendrimer-based core has 2^{x} (x is the generation number) terminal functional groups on the surface, and
among the total terminal functional groups present in the dendrimer-based core, half are bound to the peptide represented by SEQ ID NO: 1 or 2 and the other half are bound independently to the hydrophobic compound or the amino acid derivative.

17. The Janus-type dendritic polyelectrolyte zwitterion according to claim 12, wherein the positively charged peptide is represented by any one selected from a group consisting of SEQ ID NOS: 3 to 98.

18. The Janus-type dendritic polyelectrolyte zwitterion according to claim 12, wherein the negatively charged peptide is represented by any one selected from a group consisting of SEQ ID NOS: 99 to 162.

19. The Janus-type dendritic polyelectrolyte zwitterion according to claim 12, wherein the hydrophobic compound is any one selected from a group consisting of a fatty acid having 3 to 30 carbon atoms, a hydrophobic drug, and a fluorescent material.

20. A nanostructure comprising the Janus-type dendritic polyelectrolyte zwitterion according to claim 12.

21. The nanostructure according to claim 20, wherein the nanostructure is in the form of a nanofiber.

22. The nanostructure according to claim 20, wherein the average diameter of the nanostructure is 8 to 30 nm.

23. A gene or drug carrier comprising: the nanostructure according to claim 20; and a target gene or a drug loaded in the nanostructure.

24. The gene or drug carrier according to claim 23, wherein the gene is a plasmid, an mRNA, an RNA, a DNA, or a combination thereof.

25. The gene or drug carrier according to claim 23, wherein the drug is a low-molecular-weight drug, a gene drug, a protein drug, an antibody drug, a synthetic compound drug, or a combination thereof.
